SEQUENCE LISTING (23 sequences)

SEQ ID NO: 1 - DNA, 141 bp, Simian Immunodeficiency Virus
tccgtcttgtcagggaagaaagcagatgaattagaaaaaattaggctacgacccaacggaaagaaaaagtacatgttgaagcatgtagtatgggcagcaaatgaattagatagatttggattagcagaaagcctgttggag

SEQ ID NO: 2 - DNA, 123 bp, Simian Immunodeficiency Virus
tttggattagcagaaagcctgttggagaacaaagaaggatgtcaaaaaatactttcggtcttagctccattagtgccaacaggctcagaaaatttaaaaagcctttataatactgtctgcgtc

SEQ ID NO: 3 - DNA, 144 bp, Simian Immunodeficiency Virus
aaaagcctttataatactgtctgcgtcatctggtgcattcacgcagaagagaaagtgaaacacactgaggaagcaaaacagatagtgcagagacacctagtggtggaaacaggaacaacagaaactatgccaaaaacaagtaga

SEQ ID NO: 4 - DNA, 135 bp, Simian Immunodeficiency Virus
ggtaactatgtccacctgccattaagcccgagaacattaaatgcctgggtaaaattgatagaggaaaagaaatttggagcagaagtagtgccaggatttcaggcactgtcagaaggttgcaccccctatgacatt

SEQ ID NO: 5 - DNA, 243 bp, Simian Immunodeficiency Virus
tcagaaggttgcaccccctatgacattaatcagatgttaaattgtgtgggagaccatcaagcggctatgcagattatcagagatattataaacgaggaggctgcagattgggacttgcagcacccacaaccagctccacaacaaggacaacttagggagccgtcaggatcagatattgcaggaacaactagttcagtagatgaacaaatccagtggatgtacagacaacagaaccccatacca

SEQ ID NO: 6 - DNA, 498 bp, Simian Immunodeficiency Virus
atgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagtttaagagcagaacagacagatgcagcagtaaagaattggatgactcaaacactgctgattcaaaatgctaacccagattgcaagctagtgctgaaggggctgggtgtgaatcccaccctagaagaaatgctgacggcttgtcaaggagtaggggggccgggacagaaggctagattaatggcagaagccctgaaagaggccctcgcaccagtgccaatcccttttgcagcagcccaacagaggggaccaagaaagccaattaagtgttggaattgtgggaaagagggacactctgcaaggcaatgcagagccccaagaagacagggatgctgg

SEQ ID NO: 7 - DNA, 330 bp, Simian Immunodeficiency Virus
atggaggaggaaaagaggtggatagcagttcccacatggaggataccggagaggctagagaggtggcatagcctcataaaatatctgaaatataaaactaaagatctacaaaaggtttgctatgtgccccattttaaggtcggatgggcatggtggacctgcagcagagtaatcttcccactacaggaaggaagccatttagaagtacaagggtattggcatttgacaccagaaaaagggtggctcagtacttatgcagtgaggataacctggtactcaaagaacttttggacagatgtaacaccaaactatgcagacattttactgcat

SEQ ID NO: 8 - DNA, 339 bp, Simian Immunodeficiency Virus
ccaaactatgcagacattttactgcatagcacttatttcccttgctttacagcgggagaagtgagaagggccatcaggggagaacaactgctgtcttgctgcaggttcccgagagctcataagtaccaggtaccaagcctacagtacttagcactgaaagtagtaagcgatgtcagatcccagggagagaatcccacctggaaacagtggagaagagacaataggagaggccttcgaatggctaaacagaacagtagaggagataaacagagaggcggtaaaccacctaccaagggagctaattttccaggtttggcaaaggtcttgggaatactggca

SEQ ID NO: 9 - DNA, 498 bp, Simian Immunodeficiency Virus
ggattagacaagggcttgagctcactctcttgtgagggacagaaatacaatcagggacagtatatgaatactccatggagaaacccagctgaagagagagaaaaattagcatacagaaaacaaaatatggatgatatagatgaggaagatgatgacttggtaggggtatcagtgaggccaaaagttcccctaagaacaatgagttacaaattggcaatagacatgtctcattttataaaagaaaaggggggactggaagggatttattacagtgcaagaagacatagaatcttagacatatacttagaaaaggaagaaggcatcataccagattggcaggattacacctcaggaccaggaattagatacccaaagacatttggctggctatggaaattagtccctgtaaatgtatcagatgaggcacaggaggatgaggagcattatttaatgcatccagctcaaacttcccagtgggatgacccttggggagaggtt

SEQ ID NO: 10 - protein, 47 aa, Simian Immunodeficiency Virus
SVLSGKKADELEKIRLRPNGKKKYMLKHVVWAANELDRFGLAESLLE

SEQ ID NO: 11 - protein, 41 aa, Simian Immunodeficiency Virus
FGLAESLLENKEGCQKILSVLAPLVPTGSENLKSLYNTVCV

SEQ ID NO: 12 - protein, 48 aa, Simian Immunodeficiency Virus
KSLYNTVCVIWCIHAEEKVKHTEEAKQIVQRHLVVETGTTETMPKTSR

SEQ ID NO: 13 - protein, 45 aa, Simian Immunodeficiency Virus
GNYVHLPLSPRTLNAWVKLIEEKKFGAEVVPGFQALSEGCTPYDI

SEQ ID NO: 14 - protein, 81 aa, Simian Immunodeficiency Virus
SEGCTPYDINQMLNCVGDHQAAMQIIRDIINEEAADWDLQHPQPAPQQGQLREPSGSDIAGTTSSVDEQIQWMYRQQNPIP

SEQ ID NO: 15 - protein, 166 aa, Simian Immunodeficiency Virus
MYRQQNPIPVGNIYRRWIQLGLQKCVRMYNPTNILDVKQGPKEPFQSYVDRFYKSLRAEQTDAAVKNWMTQTLLIQNANPDCKLVLKGLGVNPTLEEMLTACQGVGGPGQKARLMAEALKEALAPVPIPFAAAQQRGPRKPIKCWNCGKEGHSARQCRAPRRQGCW

SEQ ID NO: 16 - protein, 110 aa, Simian Immunodeficiency Virus
MEEEKRWIAVPTWRIPERLERWHSLIKYLKYKTKDLQKVCYVPHFKVGWAWWTCSRVIFPLQEGSHLEVQGYWHLTPEKGWLSTYAVRITWYSKNFWTDVTPNYADILLH

SEQ ID NO: 17 - protein, 113 aa, Simian Immunodeficiency Virus
PNYADILLHSTYFPCFTAGEVRRAIRGEQLLSCCRFPRAHKYQVPSLQYLALKVVSDVRSQGENPTWKQWRRDNRRGLRMAKQNSRGDKQRGGKPPTKGANFPGLAKVLGILA

SEQ ID NO: 18 - protein, 166 aa, Simian Immunodeficiency Virus
GLDKGLSSLSCEGQKYNQGQYMNTPWRNPAEEREKLAYRKQNMDDIDEEDDDLVGVSVRPKVPLRTMSYKLAIDMSHFIKEKGGLEGIYYSARRHRILDIYLEKEEGIIPDWQDYTSGPGIRYPKTFGWLWKLVPVNVSDEAQEDEEHYLMHPAQTSQWDDPWGEV

SEQ ID NO: 19 - DNA, 26 bp, Artificial
  Antisense primer for Simian Immunodeficiency Virus genome
tgtaataaatcccttccagtcccccc

SEQ ID NO: 20 - DNA, 20 bp, Artificial
  Sense primer for Simian Immunodeficiency Virus genome
cctccccctccaggactagc

SEQ ID NO: 21 - DNA, 32 bp, Artificial
  Sense primer for Simian Immunodeficiency Virus genome
tatgatagacatggagacacccttgaaggagc

SEQ ID NO: 22 - DNA, 27 bp, Artificial
  Antisense primer for Simian Immunodeficiency Virus genome
atgagacatrtctattgccaatttgta

SEQ ID NO: 23 - protein, 15 aa, Simian Immunodeficiency Virus
NATIPLFCATKNRDT

(19) United States
(12) Patent Application Publication
(10) Pub. No.: US 2011/0165190 A1
(43) Pub. Date: Jul. 7, 2011

(54) Compositions and Methods for Immunizing Against Immunodeficiency Viruses

(75) Inventors: David Ian Watkins, Arena, WI (US); Nancy Ann Wilson Schlei, Hartland, WI (US)

(73) Assignee: WISCONSIN ALUMNI RESEARCH FOUNDATION, Madison, WI (US)

(21) Appl. No.: 12/833,633

(22) Filed: Jul. 9, 2010

Related U.S. Application Data
(60) Provisional application No. 61/224,195, filed on Jul. 9, 2009.

(51) Int. Cl. A61K 39/21; A61P 37/04; A61P 31/18

(52) U.S. Cl. 424/208.1

(57) ABSTRACT

Disclosed are compositions, kits, and methods related to HIV vaccines and immunogenic compositions. Typically, the compositions include a mixture of individual and separate DNA polynucleotides that encode different HIV polypeptides or fragments thereof. The mixture of polynucleotides is designed to elicit a broad immunogenic response which may be subsequently boosted by administering a second composition such as a recombinant virus vector composition. The compositions, kits, and methods may be utilized for treating or preventing HIV infection or disease.

### CROSS-REFERENCE TO RELATED APPLICATIONS

This application claims the benefit under 35 U.S.C. §119(e) to U.S. Provisional Application No. 61/224,195, filed on Jul. 9, 2009, the content of which is incorporated herein by reference in its entirety.

### STATEMENT REGARDING FEDERALLY SPONSORED RESEARCH OR DEVELOPMENT

This invention was made with U.S. government support under grant no. R01 AI049120, awarded by the National institutes of Health. The U.S. government has certain rights in the invention.

### BACKGROUND

The present invention relates generally to the field of compositions, kits, and methods for immunizing against immunodeficiency viruses, including primate immunodeficiency viruses such as human immunodeficiency virus (HIV) and simian immunodeficiency virus (SIV). In particular, the invention relates to nucleic acid vaccines and viral vector vaccines against HIV infection and SIV infection.

A long-term solution to the human immunodeficiency virus (HIV) epidemic is a vaccine that prevents infection or substantially reduces the likelihood of transmission. Towards this goal, the HIV Vaccine Trials Network (HVTN) was established. The HVTN is a non-profit organization that includes physicians, scientists, activists, and community educators that work together to conduct clinical trials with the goal of obtaining a safe and effective HIV vaccine. In 2005, the HVTN initiated the STEP trial, which was a double-blind randomized controlled HIV vaccine trial. Participants in the study were men and women that were identified as high risk candidates for HIV infection but who currently were HIV-negative. Participants were administered three vaccinations of recombinant adenovirus that was engineered to express the HIV gag, pol, and nef polypeptides. This particular delivery vector (i.e., recombinant adenovirus) and these particular polypeptides (i.e., gag, pol, and nef) were selected with the goal of inducing a cell-mediated immune response.

In September 2007, the STEP trial was terminated when in the entire study, 49/914 participants in the vaccine group and 33/922 participants in the placebo group had tested HIV-positive. This was contrary to the desired result where researchers had expected that the group which had received the vaccine would have a lower or equal infection rate as compared to the control group. More problematic was the fact that certain groups of the vaccine recipients were seen to have higher risk of HIV infection as compared to the placebo group. By November 2007 all participants were informed whether they had received the vaccine or placebo.

The lack of vaccine efficacy in the STEP trial has led some to conclude that CTL-based vaccines may not be a viable approach to impact the AIDS epidemic. However, STEP trial vaccinees who became infected recognized a median of five (5) epitopes, mostly in the conserved proteins gag and pol. Given the sequence diversity of HIV, several of these vaccine epitopes may have been absent from the breakthrough viruses infecting vaccinees. Therefore, CTL-based vaccines that induce broad recognition of a larger number of epitopes may be more efficacious.

### SUMMARY

Disclosed are compositions, kits, and methods for inducing an immune response against HIV or SIV. Typically, the compositions include a mixture of DNA polynucleotides encoding one or more HIV or SIV polypeptides or fragments thereof. Suitable HIV or SIV polypeptides may include Gag, Pol, Rev, Tat, Nef, Vif, Vpx, Vpr, Env, Vpu, or fragments thereof (preferably Gag, Pol, Tat, Nef, Rev, Vif, Vpx, Vpr, or fragments thereof, and more preferably, at least Gag, Vif, or fragments thereof). The disclosed compositions may be utilized as pharmaceutical immunogenic compositions or vaccines that induce a cytotoxic T-cell (CTL)-based response.

In some embodiments, the compositions may include a polynucleotide or a panel of polynucleotides (e.g., at least about 2-9 polynucleotides) that encode and express a single HIV or SIV polypeptide such as full-length Gag polypeptide or one or more fragments thereof. Optionally, the compositions may include a polynucleotide or a panel of polynucleotides that encode and express a single HIV or SIV polypeptide such as full-length Vif polypeptide or one or more fragments thereof. Optionally, the compositions may include a polynucleotide or a panel of polynucleotides that encode and express a single HIV or Sly polypeptide such as full-length Nef polypeptide or one or more fragments thereof. Optionally, the compositions may include polynucleotide or a panel of polynucleotides that encode and express a single HIV or Sly polypeptide such as full-length Tat polypeptide or one or more fragments thereof. Optionally, the compositions may include polynucleotide or a panel of polynucleotides that encode and express a single HIV or SIV polypeptide such as full-length Rev polypeptide or one or more fragments thereof. Optionally, the compositions may include a polynucleotide or a panel of polynucleotides that encode and express a single HIV or SIV polypeptide such as full-length Pol polypeptide or one or more fragments thereof. Optionally, the compositions may include a polynucleotide or a panel of polynucleotides that encode and express a single HIV or SIV polypeptide such as full-length Vpr polypeptide or one or more fragments thereof. Optionally, the compositions may include a polynucleotide or a panel of polynucleotides that encode and express a single HIV or SIV polypeptide such as full-length Vpx polypeptide or one or more fragments thereof. The polynucleotides or panels of polynucleotides typically encode different polypeptides or different fragments of polypeptides. Fragments typically range in length from 5-200 amino acids (or from 5-150, 5-100, 5-50, 5-25, 5-15, 10-200, 10-100, 10-50, 10-25, 10-25, or 10-15 amino acids) and include at least one epitope of the polypeptide from which the fragment is derived.

In some embodiments, the pharmaceutical composition comprises a mixture of DNA polynucleotides and a pharmaceutical carrier (e.g., an excipient, diluent, adjuvant, or combination thereof). The mixture or the composition may include: (a) a first polynucleotide (or a first panel of polynucleotides) encoding and capable of expressing a single HIV or SIV polypeptide such as an amino acid sequence of HIV or SIV Gag polypeptide; and (b) a second polynucleotide (or a second panel of polynucleotides) encoding and capable of expressing a single HIV or SIV polypeptide such as an amino acid sequence of a polypeptide selected from the group consisting of HIV or SIV Pol, Tat, Nef, Rev, Vif, Vpr, and Vpx polypeptide; where the polynucleotides of the mixture are separate polynucleotides (or the panels of the mixture comprise separate polynucleotides) and each do not encode the same HIV amino acid sequence and/or each encode a unique amino acid sequence. In one example, the mixture of the pharmaceutical composition may comprise: (a) a polynucleotide encoding and capable of expressing a single HIV or SIV polypeptide such as an amino acid sequence of HIV or SIV Gag polypeptide; optionally, (b) an additional polynucleotide encoding and capable of expressing a single HIV or SIV polypeptide such as an amino acid sequence of HIV Vif polypeptide; optionally, (c) an additional polynucleotide encoding and capable of expressing a single HIV or Sly polypeptide such as an amino acid sequence of HIV or SIV Nef polypeptide; optionally, (d) an additional polynucleotide encoding and capable of expressing a single HIV or SIV polypeptide such as an amino acid sequence of HIV or SIV Tat polypeptide; optionally, (e) an additional polynucleotide encoding and capable of expressing a single HIV or SIV polypeptide such as an amino acid sequence of HIV or SIV Rev polypeptide; optionally, (f) an additional polynucleotide encoding and capable of expressing a single HIV or SIV polypeptide such as an amino acid sequence of HIV or SW Pol polypeptide; optionally, (g) an additional polynucleotide encoding and capable of expressing a single HIV or Sly polypeptide such as an amino acid sequence of HIV or SW Vpr polypeptide; and optionally, (h) an additional polynucleotide encoding and capable of expressing a single HIV or SIV polypeptide such as an amino acid sequence of SIV Vpx polypeptide. The pharmaceutical composition may or may not comprise a DNA polynucleotide that encodes and is capable of expressing a single HIV or SIV polypeptide consisting of full-length HIV or SIV Env polypeptide.

The DNA polynucleotides of the pharmaceutical composition may encode and express a single HIV or SIV polypeptide such as an HIV or SIV polypeptide or one or more fragments thereof, and optionally may encode and express an additional non-HIV or non-SIV polypeptide or one or more fragments thereof. In some embodiments, the mixture of the composition includes a first polynucleotide that encodes and expresses a single HIV or SIV polypeptide consisting of an amino acid sequence of HIV or SIV Gag polypeptide (e.g., either the full-length HIV or SIV Gag polypeptide or one or more fragments comprising or consisting of 5-200 amino acids of the HIV or SIV Gag polypeptide (or one or more fragments comprising or consisting of 5-150, 5-100, 5-50, 5-25, 5-15, 10-200, 10-150, 10-100, 10-50, 10-25, 10-25, or 10-15 amino acids of the HIV or SIV Gag polypeptide)). The mixture of the composition optionally may include an additional polynucleotide encoding and expressing a single HIV or Sly polypeptide consisting of an amino acid sequence of HIV or SIV Vif polypeptide (e.g., either the full-length HIV or SIV Vif polypeptide or one or more fragments comprising or consisting of 5-150 amino acids of the HIV or SIV Vif polypeptide (or one or more fragments comprising or consisting of 5-100, 5-50, 5-25, 5-15, 10-150, 10-100, 10-50, 10-25, or 10-15 amino acids of the HIV or SIV Vif polypeptide)). The mixture of the composition optionally may include an additional polynucleotide encoding and expressing a single HIV or SIV polypeptide consisting of an amino acid sequence of HIV or SIV Nef polypeptide (e.g., either the full-length HIV or SIV Nef polypeptide or one or more fragments comprising or consisting of 5-200 amino acids of the HIV or SIV Nef polypeptide (or one or more fragments comprising or consisting of 5-150, 5-100, 5-50, 5-25, 5-15, 10-200, 10-150, 10-50, 10-25, or 10-15 amino acids of the HIV or SIV Nef polypeptide)). The mixture of the composition optionally may include an additional polynucleotide encoding and expressing a single HIV or SIV polypeptide consisting of an amino acid sequence of HIV or SW Tat polypeptide (e.g., either the full-length HIV or SIV Tat polypeptide or one or more fragments comprising or consisting of 5-100 amino acids of the HIV or SIV Tat polypeptide (or one or more fragments comprising or consisting of 5-50, 5-25, 5-15, 10-100, 10-50, 10-25, or 10-15 amino acids of the HIV or SIV Tat polypeptide)). The mixture of the composition optionally may include an additional polynucleotide encoding and expressing a single HIV or SIV polypeptide consisting of an amino acid sequence of HIV Rev polypeptide (e.g., either the full-length HIV Rev polypeptide or one or more fragments comprising or consisting of 5-100 amino acids of the HIV Rev polypeptide (or one or more fragments comprising or consisting of 5-50, 5-25, 5-15, 10-100, 10-50, 10-25, or 10-15 amino acids of the HIV Rev polypeptide)). The mixture of the composition optionally may include an additional polynucleotide encoding and expressing a single HIV or Sly polypeptide consisting of an amino acid sequence of HIV or SIV Pol polypeptide (e.g., either the full-length HIV or Sly Pol polypeptide or one or more fragments comprising or consisting of 5-200 amino acids of the HIV or SIV Pol polypeptide (or one or more fragments comprising or consisting of 5-150, 5-100, 5-50, 5-25, 5-15, 10-200, 10-150, 10-100, 10-50, 10-25, or 10-15 amino acids of the HIV or SIV Pol polypeptide)). The mixture of the composition optionally may include an additional polynucleotide encoding and expressing a single HIV or SIV polypeptide consisting of an amino acid sequence of HIV or SIV Vpr polypeptide (e.g., either the full-length HIV or SIV Vpr polypeptide or one or more fragments comprising or consisting of 5-100 amino acids of the HIV or SIV Vpr polypeptide (or one or more fragments comprising or consisting of 5-50, 5-25, 5-15, 10-100, 10-50, 10-25, or 10-15 amino acids of the HIV or SIV Vpr polypeptide)). The mixture of the composition optionally may include an additional polynucleotide encoding and expressing a single HIV or SIV polypeptide consisting of an amino acid sequence of SIV Vpx polypeptide (e.g., either the full-length SIV Vpx polypeptide or one or more fragments comprising or consisting of 5-100 amino acids of the SIV Vpx polypeptide (or one or more fragments comprising or consisting of 5-50, 5-25, 5-15, 10-50, 10-25, or 10-15 amino acids of the SIV Vpx polypeptide)).

Typically, the pharmaceutical composition comprises an effective amount or concentration of the mixture of polynucleotides for inducing a protective or therapeutic immune response against HIV infection in humans or SIV infection in simians. Inducing a protective or therapeutic immune response may include potentiating a CD8+ response to one or more HIV or SIV epitopes. Inducing a protective response may include inducing sterilizing immunity against HIV or SIV. Inducing a therapeutic response may include reducing the viral load of a subject, for example, as determined by measuring the amount of circulating virus before and after administering the composition. In some embodiments, the viral load is reduced to less than about 10,000 vRNA copies/ml (preferably less than about 5,000 vRNA copies/ml, more preferably less than about 2000 vRNA copies/ml, even more preferably less than about 1500 vRNA copies/ml, and most preferably less than about 1000 vRNA copies/ml) after at least about 3-months post-administering the composition. Inducing a therapeutic response may include increasing the CD4⁺ T-cell count of the subject (e.g., by at least 2 times, 3 times, or 4 times) after at least about 3-months post-administering the composition.

The pharmaceutical composition typically includes a mixture of polynucleotides, which may be present in a nucleotide vector such as a plasmid or a shuttle vector. In some embodiments, the polynucleotides are present in nucleotide vectors for expressing the encoded polypeptides, such as expression vectors. Suitable nucleotide vectors may include, but are not limited to, viral nucleotide vectors (e.g., viral subgenomic vectors) and bacterial nucleotide vectors (e.g., plasmids or bacteriophage vectors). The pharmaceutical composition may include naked recombinant DNA or recombinant DNA packaged in one or more virus particles (e.g., replication defective virus particles). In some embodiments, the polynucleotides are present in one or more virus or bacterial nucleotide vectors packaged in one or more recombinant viruses or bacteria. For example, the polynucleotides may be present in one or more adenovirus DNA vectors packaged in one or more recombinant adenoviruses. In further embodiments, the polynucleotides are present in one or more Sendai or measles virus nucleotide vectors packaged in one or more recombinant Sendai or measles viruses. In other embodiments, the polynucleotides may be present in a recombinant bacteria that expresses the encoded polypeptides as a live bacterial vaccine (e.g., recombinant attenuated Shigella, Salmonella, Listeria, or Yersinia).

Also disclosed are methods for inducing a protective or therapeutic immune response against HIV or Sly infection by administering the pharmaceutical compositions disclosed herein (e.g., as immunogenic compositions or vaccines) to a subject in need thereof, which may include a human having or at risk for acquiring an HIV infection or a simian having or at risk for acquiring an SIV infection. The methods may include administering a first pharmaceutical composition and optionally administering a second pharmaceutical composition to augment or boost an immunogenic response induced by the first pharmaceutical composition. The first and second pharmaceutical compositions may be the same or different. In some embodiments, the method may include: (a) administering a first pharmaceutical composition that comprises a mixture of DNA polynucleotides (optionally naked DNA polynucleotide) that encode and express a single HIV or SIV polypeptide or a fragment thereof (e.g., Gag, Rev, Tat, Nef, Vif, Pol, Vpr, Vpx, or fragments thereof); and (b) administering a second pharmaceutical composition that comprises packaged recombinant virus vectors (e.g., adenovirus vectors, Sendai virus vectors, or measles virus vectors) or bacterial vectors (e.g. recombinant attenuated Shigella, Salmonella, Listeria, or Yersinia bacteria) that express a single HIV or SIV polypeptide or one or more fragments thereof (e.g., Gag, Rev, Tat, Nef, Vif, Pol, Vpr, Vpx, or fragments thereof). The second pharmaceutical composition may be administered prior to, concurrently with, or after administering the first pharmaceutical composition. In some embodiments, the first composition is administered more than once (e.g., administered 2 times or 3 times with 2, 3, or 4 weeks between administrations) and subsequently the second composition is administered one or more times (e.g., administered 2 times or 3 times with 2, 3, or 4 weeks between administrations). Where the second composition is administered after the first composition, the second composition may be administered 1, 2, 3, 4, 5, or 6 months after the first composition is administered.

Also disclosed are kits that may include the pharmaceutical compositions disclosed herein or kits that may be used to prepare the pharmaceutical compositions disclosed herein. For example, the kits may include individual DNA polynucleotides as disclosed herein that may be combined to form the mixture of the pharmaceutical compositions or vectors that comprise the DNA polynucleotides as disclosed herein that may be combined to form the mixture of the pharmaceutical compositions. The kits may be used to practice the methods disclosed herein and may include as components: the pharmaceutical compositions disclosed herein, additional therapeutic or prophylactic agents, and/or implements for administering the kit components.

### BRIEF DESCRIPTION OF THE DRAWINGS

FIG. 1. Vaccination scheme and vectors. Eight rhesus macaques, expressing the MHC class I allele Mamu-A*02, (all Mamu-A*01 negative, Mamu-B*08 negative and Mamu-B*17 negative), were primed three times at four week intervals with DNA encoding each of the eight SIV proteins except Env. Animals were boosted at week 24 with six Ad5 vectors, encoding the same protein sequences as the DNA prime; one of the Ad5 vectors contained three of the SIV proteins in a single open reading frame (vif, vpr and vpx). Thirty-seven weeks later, the vaccinees and 8 na\ddot{v}e control animals which also expressed Mamu-A*02 and were negative for Mamu-A*01, -B*08 and -B*17 (in gray) were challenged intrarectally with SIVsmE660 (800 TCID₅₀, 1.2×10⁷ copy Eq). If animals were not productively infected (>2 consecutive positive viral loads) following a given challenge, they were challenged again three weeks later, up to a total of five low dose challenges. If animals were not productively infected by 5 low dose challenges, they were challenged with up to six high dose challenges every two weeks with SIVsmE660 (4,000 TCID₅₀, 6×10⁷ vRNA copy Eq). As with the previous dose, once an animal had two or more consecutive positive viral loads, it was considered to be infected and not challenged further.

FIG. 2. Plasma virus concentrations after SIVsmE660 infection. Vaccinees (A) and Controls (B) were infected after up to 5 low dose mucosal challenges and up to 6 high dose challenges (See Table 3). The upper horizontal line on each graph indicates a viral load of 10⁶ copy Eq/ml, and the lower horizontal line is the limit of detection for the viral load assay (30 copy Eq/ml). Animals receiving high doses are indicated with an asterisk after the animal ID. Animal r02012 had extremely high viral loads and was sent to necropsy after 10 weeks of infection, as indicated by the symbol (=). C. Means of the viral load for infected animals. This is the graphical representation of the data presented in Table 4. Chronic viral loads were determined for the range from 6 weeks to 20 weeks post infection.

FIG. 3. Representative anamnestic vaccine-induced immune responses in the vaccinees. All vaccinees made CD8⁺ T-cell responses to several epitopes, including some restricted by the MHC class I molecule Mamu-A*02. Two of the vaccinees are shown in this figure, and data from the remaining vaccinees is presented in FIG. 7. Responses post-challenge are indicated in dark, black bars, and responses observed immediately prior to challenge are indicated in lighter, gray bars. Whole peripheral blood mononuclear cell (PBMC) responses are indicated as are responses in PBMC depleted of CD8+ cells. Responses in PBMC depleted of CD8+ cells are likely mediated by CD4⁺ T cells. CD8⁺ cell depletion was typically 99% complete (data not shown). Responses to minimal optimal peptides that bind to Mamu-A*02 also are shown. As indicated in Table 7, some of these epitopes are conserved between SIVmac239 and SIVsmE660, whereas others have several substitutions, some of which could affect T-cell recognition. Most anamnestic response analyses were performed at 14-15 days post infection. For a couple of animals (r00061, r02103), these assays were delayed to 21 days post infection due to the very low viral loads observed.

FIG. 4. Western blot at 8 weeks post vaccination. Serum samples were collected at 8 weeks after the Ad5 boost. The ZeptoMetrix SIV Western Blot Assay kit was used to visualize antibodies to Env, Gag and Pol. Antibody was allowed to bind to the blot overnight, which has been shown to be more sensitive.

FIG. 5. Viral replication in titration animals. The upper horizontal line on each graph indicates a viral load of 10⁶ copy Eq/ml, and the lower horizontal line is the limit of detection for the viral load assay (30 copy Eq/ml).

FIG. 6. Absolute CD4 and CD4 total memory counts. PBMC were isolated from fresh EDTA-anticoagulated blood using a Ficoll density gradient, then stained for CD3 (Alexa 700), CD4 (PerCP), CD8 (Pacific Blue), CD28 (PE), CD95 (FITC) and β7 integrin (APC). CBCs were performed on whole, EDTA-anticoagulated blood. A. CD4 counts did not vary much between vaccinees (black) and controls (gray). Geometric means are not significantly different between these two groups. B. CD4 memory counts were numerically higher in vaccinees than in controls, but this difference is not statistically significant.

FIG. 7. Anamnestic vaccine-induced immune responses in the vaccinees. All vaccinees made CD8⁺ T-cell responses to several epitopes, including some restricted by the MHC class I molecule Mamu-A*02. Two of the vaccinees are shown in FIG. 4, and data from the remaining vaccinees is presented in this figure. Responses post-challenge are indicated in dark, black bars, and responses observed immediately prior to challenge are indicated in lighter, gray bars. Whole PBMC responses are indicated as are responses in PBMC depleted of CD8⁺ cells. Responses in PBMC depleted of CD8+ cells are likely mediated by CD4⁺ T cells. CD8⁺ cell depletion was typically 99% complete (data not shown). Responses to minimal optimal peptides that bind to Mamu-A*02 also are shown. As indicated in Table 7, some of these epitopes are conserved between SIVmac239 and SIVsmE660, whereas others have several substitutions, some of which could affect T-cell recognition. Most anamnestic response analyses were performed at 14-15 days post infection. For a couple animals (r00061, r02103), these assays were delayed to 21 days post infection due to the very low viral loads observed.

FIG. 8. Ten naïve animals challenged i.v. with this stock of SIVsmE660 all had viral loads above 100,000 vRNA copy Eq/ml at 28 weeks post infection. Two of these animals were Mamu-A*01 positive, two were Mamu-B*08 positive and two were Mamu-B*17 positive.

FIG. 9. Five of the delta-Nef vaccinees showed some control over replication of SIVsmE660 (vaccinees in black, naïve controls in gray). The majority of the animals showing control were Mamu-A*01, -B*08 or -B*17 positive.

FIG. 10. Log transformed means for naïve animals infected i.v. (dashed line) compared to naïve animals infected i.r. in this experiment (solid line) shows that while the animals infected i.r. have slightly lower viral loads, this difference is not significant (P<0.1069), by the unpaired Student's t-test.

FIG. 11. Illustrates that a preferable goal of a T-cell based vaccine is to prevent transmission in the chronic phase by reducing viral load (e.g., to less than ˜1,500 vRNA copies/trip.

FIG. 12. VSA schematic. A) PBMC-derived CD8-depleted target cells were activated with Concanavalin A (5 μg/ml) for 18-24 hours. B) Using part of the PBMC obtained on day −2, effectors (CD3+CD8+ T-cells) were generated by bead-depletion of non-CD3+CD8+ cells. C) Target cells were infected with SIVmac239 or SIVsmE660 and then mixed effectors and targets at E:T ratios of 0.1:1, 0.5:1, and 1:1. D) The co-cultures were maintained for 7 days, and then intracellular staining of SIV Gag was performed. The percentage of maximum suppression were evaluated by comparing the frequency of SIV Gag+ cells in the presence or absence of effectors.

FIG. 13. Suppression of SIVmac239 replication by vaccine-induced CD8+T-cells. Mean percentages of maximum suppression of SIVmac239 replication of individual animals in the vaccinated (A) and control (B) groups at 3 E:T ratios, based on 2 independent experiments. C) After testing for normality and homogeneity of variances, the mean percentages of maximum suppression from both vaccinated and control groups were averaged as described in the Materials and Methods section. Then, both groups at each E:T ratio were compared. Error bars represent standard error of the mean. *p<0.05 according to the Student's t-test.

FIG. 14. Suppression of SIVsmE660 replication by vaccine-induced CD8+T-cells. Mean percentages of maximum suppression of SIVsmE660 replication of individual animals in the vaccinated (A) and control (B) groups at 3 E:T ratios, based on 2 independent experiments. C) After testing for normality and homogeneity of variances, the mean percentages of maximum suppression from both vaccinated and control groups were averaged as described in the Materials and Methods section. Then, both groups at each E:T ratio were compared. Error bars represent standard error of the mean. *p<0.05 according to the Wilcoxon assigned-rank test.

FIG. 15. Comparison between in vitro suppression of SIVsmE660 replication and markers of disease progression. Each symbol represents the mean percentage of maximum suppression from one vaccinee plotted against its corresponding viral load (VLs) (A and B) and absolute CD4+ T-cell counts (C and D). In vitro suppression of viral replication compared to peak VLs (A), setpoint VLs (B), absolute CD4+ T-cell counts in the acute phase (weeks 2-3 post infection) (C), and absolute CD4+ T-cell counts in the chronic phase (week 24 post infection) (D). Correlation coefficients (r) and p values were determined using the Spearman's rank correlation test.

FIG. 16. Correlations between the magnitude of vaccine-induced T-cell responses and markers of disease progression. Each symbol represents the magnitude of IFN-γ+ responses from one vaccinee measured before (A, C, and D) or after (B, E, and F) infection. A) Total magnitude of pre-challenge vaccine-induced T-cell responses compared to peak (left panel) and setpoint (right panel) viral loads (VLs). B) Total magnitude of post-challenge vaccine-induced T-cell responses compared to peak (left panel) and setpoint (right panel) viral loads. C) Pre-challenge response to Vpr compared to setpoint viral loads. D) Pre-challenge response to Rev compared to absolute CD4+ T-cell counts in the acute phase (weeks 2-3 post infection). E) Post-challenge anamnestic response to Nef compared to setpoint viral loads. D) Post-challenge anamnestic CD4+ T-cell response to Pol compared to absolute CD4+ T-cell counts in the chronic phase (week 24 post infection). Correlation coefficients (r) and p values were determined using the Spearman's rank correlation test. SFC: spot-forming cells.

FIG. 17. Correlations between the epitope breadth of vaccine-induced T-cell responses and markers of disease progression. Each symbol represents the epitope breadth of T-cell responses from one vaccinee measured before (A, C, D, and E) or after (B) infection. A) Total breadth of pre-challenge vaccine-induced T-cell responses compared to peak (left panel) and setpoint (right panel) viral loads (VLs). B) Total breadth of post-challenge vaccine-induced T-cell responses compared to peak (left panel) or setpoint (right panel) viral loads. C) Pre-challenge Vif epitope breadth compared to peak VLs. D) Pre-challenge Vif epitope breadth compared to absolute CD4+ T-cell counts in the chronic phase (week 24 post infection). E) Pre-challenge Gag epitope breadth compared to setpoint viral loads. Correlation coefficients (r) and p values were determined using the Spearman's rank correlation test.

FIG. 18. Correlation between suppression of SIVmac239 and SIVsmE660 replication among vaccinees at E:T ratios of 0.1:1 (A), 0.5:1 (B), and 1:1 (C). Correlation coefficients (r) and p values were determined using the Spearman's rank correlation test.

FIG. 19. Exemplary fragments of Gag, Vif, and Nef polypeptides for use in the presently disclosed compositions.

### DETAILED DESCRIPTION

Disclosed are composition, kits, and methods for treating or preventing infection with human immunodeficiency virus (HIV) or simian immunodeficiency virus (SIV). In particular, the composition, kits, and methods relate to HIV or SIV immunogenic compositions and HIV or SIV vaccines that include mixtures of individual and separate DNA polynucleotides, whereby HIV or SIV polypeptides or fragments thereof are encoded on the individual and separate polynucleotides and expressed therefrom. Related compositions, kits, and methods are disclosed in U.S. patent application Ser. No. 12/022,530, filed on Jan. 30, 2008, which claims the benefit of U.S. Provisional Patent Application No. 60/898,644, filed on Jan. 31, 2007, the contents of which are incorporated herein by reference in their entireties. Related compositions, kits, and methods are also disclosed in Wilson et al., “Vaccine-Induced Cellular Immune Responses Reduce Viral Concentrations after Repeated Low-Dose Challenge with Pathogenic Simian Immunodeficiency Virus SIVmac239,” J. Virol., June 2006, p. 5875-5886; and Reynolds et al., “Macaques vaccinated with live-attenuated SIV control replication of heterologous virus,” J. Exp. Med. 2007, Vol. 205, No. 11, 2537-2550; which are incorporated herein by reference in their entireties.

### Definitions

The present invention is described herein using definitions, as set forth below and throughout the application.

Unless otherwise specified or indicated by context, the terms “a,” “an,” and “the,” mean “one or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≦10% of the particular term and “substantially” and “significantly” will mean plus or minus >10% of the particular term.

As used herein, the terms “include” and “including” have the same meaning as the terms “comprise” and “comprising.”

A “subject,” “patient,” or “host” refers to a human or non-human primate having, or at risk for acquiring, a primate lentivirus infection (e.g., HIV-1, HIV-2, or SIV). A “subject,” “patient,” or “host” may be a human. Individuals who are treated with the pharmaceutical compositions disclosed herein may be at risk for infection with the virus or may have already been infected. The terms “subject,” “patient,” or “host” may be used interchangeably.

As used herein “HIV” refers to “human immunodeficiency virus” which may include human immunodeficiency virus type 1 (i.e., “HIV-1”) and human immunodeficiency virus type 2 (i.e., “HIV-2”). As used herein “SIV” refers to “simian immunodeficiency virus.”

As used herein, a “CD8⁺ response” is referred to as the ability of cytotoxic CD8⁺ T-cells to recognize and kill cells expressing foreign peptides in the context of a major histocompatibility complex (MHC) class I molecule.

As used herein, “potentiating” or “enhancing” an immune response means increasing the magnitude and/or the breadth of the immune response. For example, the number of cells that recognize a particular epitope may be increased (“magnitude”) and/or the numbers of epitopes that are recognized may be increased (“breadth”). Preferably, a 5-fold, or more preferably a 10-fold or greater, enhancement in CD8⁺ and/or CD4⁺ T-cell responses may be obtained by administering the pharmaceutical composition disclosed herein.

As used herein, “viral load” is the amount of virus present in the blood of a patient. Viral load is also referred to as viral titer or viremia. Viral load can be measured in variety of standard ways including copy Equivalents of the viral RNA (vRNA) genome per milliliter blood plasma (vRNA copy Eq/ml). This quantity may be determined by standard methods that include RT-PCR. In preferred embodiments, the pharmaceutical composition disclosed herein, after being administered to a subject in need thereof, result in a reduction in the viral load in said subject (e.g., to no more than about 5000 copies vRNA/ml, preferably to no more than about 2000 copies vRNA/ml, more preferably to no more than about 1000 copies vRNA/ml).

As used herein, “structural viral proteins” are those proteins that are physically present in the virus. They include the capsid and matrix proteins (e.g., those proteins encoded by Gag) and enzymes that are packaged into the capsid with the genetic material.

As used herein, “non-structural viral proteins” are those proteins that are needed for production of live virus but are not necessarily found as components of the viral particle (e.g., Tat, Rev, Vif, Nef, and Vpr). They include DNA binding proteins and enzymes that are encoded by viral genes but which are not present in the virions. Proteins are meant to include both the intact proteins and fragments of the proteins or peptides which are recognized by the immune cell as epitopes of the native protein.

The compositions disclosed herein include polynucleotides that may encode full-length HIV or SIV polypeptides or fragments thereof. “Minigenes” that encode for only a portion of a gene are described in U.S. patent application Ser. No. 12/022,530, filed on Jan. 30, 2008, which claims the benefit of U.S. Provisional Patent Application No. 60/898,644, filed on Jan. 31, 2007, the contents of which are incorporated herein by reference in their entireties. For example, a minigene for HIV Gag may encode for only 5-200 amino acids of the Gag polypeptide (or 5-150, 10-150, 5-100, 10-100, 5-50, 10-50, 5-25, 10-25, 5-15, or 10-15 amino acids).

A “fragment” as contemplated herein refers to a contiguous portion of a nucleic acid or amino acid reference sequence. A fragment of a reference polynucleotide refers to less than a full-length nucleic acid sequence of the reference polynucleotide (e.g., where the reference polynucleotide is truncated at the 5′-terminus, 3′-terminus, or both termini). For example, a fragment of a reference polynucleotide may comprise or consist of a 15-600, 15-450, 15-300, 15-150, 15-75, 15-45, 30-600, 30-450, 30-300, 30-150, 30-75, or 30-45 contiguous nucleic acid sequence of the full-length reference polynucleotide. A fragment of a reference polypeptide refers to less than a full-length amino acid sequence of the reference polypeptide (e.g., where the reference polypeptide is truncated at the N-terminus, the C-terminus, or both termini). For example, a fragment of a reference polypeptide may comprise or consist of a 5-200, 5-150, 5-100, 5-50, 5-25, 5-15, 10-200, 10-150, 10-100, 10-50, 10-25, or 10-15 contiguous amino acid sequence of the full-length reference polypeptide. An “immunogenic fragment” of a polypeptide is a fragment of a polypeptide typically at least 5 or 10 amino acids in length that includes one or more epitopes of the full-length polypeptide.

The composition disclosed herein may include a polynucleotide or a mixture of polynucleotides that encode and express a single HIV or SIV polypeptide or a fragment thereof. For example, the polynucleotides contemplated herein may comprise a single HIV or Sly open reading frame (and optionally may include one or more non-HIV or non-SIV open reading frames). For example, a suitable polynucleotide for the disclosed compositions may encode and express a single HIV or SIV polypeptide consisting of full-length Gag, Vif, Nef, Tat, Rev, Pol, Vpr, Vpx, Vpu, or Env polypeptide. A suitable polynucleotide for the disclosed composition may encode and express a single HIV or SIV polypeptide consisting of a fragment of Gag, Vif, Nef, Tat, Rev, Pol, Vpr, Vpx, Vpu, or Env polypeptide, where the fragment is truncated at the N-terminus, the C-terminus, or both termini relative to the full-length polypeptide. Fragments of HIV or SIV polypeptides that are truncated at the N-terminus relative to the full-length polypeptide may be fused in-frame at the N-terminus to an ATG codon encoding a methionine residue and optionally one or more additional codons in order to provide a start codon for the open reading frame (ORF) of the fragment (e.g., 5′-ATG-NNN-NNN-(HIV or SIV polypeptide fragment ORF)-3′, which encodes MXX-(HIV or SIV polypeptide fragment) where “MXX” is a not naturally present amino acid sequence). Fragments of HIV or SIV polypeptides that are truncated at the C-terminus relative to the full-length polypeptide may be fused in-frame at the C-terminus to a stop codon and optionally one or more additional codons in order to provide a stop codon for the open reading frame (ORF) of the fragment (e.g., 5′-(HIV or SIV polypeptide fragment ORF)-NNN-NNN-(stop codon)-3′, which encodes (HIV or SIV polypeptide fragment)-XXX where “XXX” is a not naturally present amino acid sequence).

An open reading frame (ORF) as contemplated herein includes a nucleic acid sequence encoding an amino acid sequence, and typically an amino acid sequence of at least about 10, 15, 25, 50, 100, 150, or 200 amino acids, where the nucleic acid sequence encodes a start codon at the 5′-terminus and a stop codon at the 3′-terminus. An ORF that encodes an HIV or SIV polypeptide or a fragment thereof typically encodes at least about a 10, 15, 25, 50, 100, 150, or 200 contiguous amino acid sequence of the HIV or SIV polypeptide which optionally may be fused in-frame at the N-terminus, the C-terminus, or both termini to one or more not naturally present amino acids (e.g., XXX-(HIV or SIV polypeptide or fragment thereof), (HIV or Sly polypeptide or fragment thereof)-XXX, or XXX-(HIV or SIV polypeptide or fragment thereof)-XXX), where “X” is a not naturally present amino acid).

As used herein, a “panel” of polynucleotides means two or more separate and different polynucleotides. A panel of polynucleotides may encode different or unique amino acid sequences of a selected polypeptide.

A “nucleic acid vaccine” or “naked DNA vaccine” refers to a vaccine that includes one or more expression vectors that encodes T-cell epitopes and/or B-cell epitopes and provides an immunoprotective response in the person being vaccinated. Nucleic acid vaccines as defined herein, which may include plasmid expression vectors, typically are not encapsidated in a viral particle. The nucleic acid vaccine is directly introduced into the cells of the individual receiving the vaccine regimen. This approach is described, for instance, in U.S. Pat. Nos. 5,580,859; 5,589,466; 5,804,566; 5,739,118; 5,736,524; 5,679,647; and WO 98/04720. Examples of DNA-based delivery technologies may include, “naked DNA” delivery, facilitated delivery (e.g., via use of bupivicaine, polymers, or peptides), and cationic lipid complex or liposome delivery. The nucleic acids can be administered using ballistic delivery (e.g., as described in U.S. Pat. No. 5,204,253) or pressure-based delivery (e.g., as described in U.S. Pat. No. 5,922,687).

The nucleic acids utilized in the compositions and vaccines disclosed herein may encode one or more viral proteins or portions of one or more viral proteins. In some embodiments, the nucleic acids may encode an epitope (e.g., a T cell epitope) that is not part of a known HIV or SIV functional protein or that is encoded by an alternate HIV or SIV open reading frame (i.e., a “cryptic” epitope). (See, e.g., Maness et al., J. Exp. Med. (2007), 204:2505-12; and Cardinaud et al., J. Exp. Med. (2004), 199:1053-1063, which are incorporated by reference herein in their entirety). A cryptic epitope may be located in an open reading frame (ORF) that is not typically observed to be translated during the course of HIV or SIV viral replication. For example, Maness et al. disclose a cryptic epitope in SIVmac239 referred to as “cRW9” which is located in the +2 reading frame relative to the ORF encoding the envelope protein. This cryptic epitope is located in the same ORF that encodes exon 1 of the Rev protein but is downstream of the only known splice donor site and so is not predicted to be translated under “normal” biological circumstances.

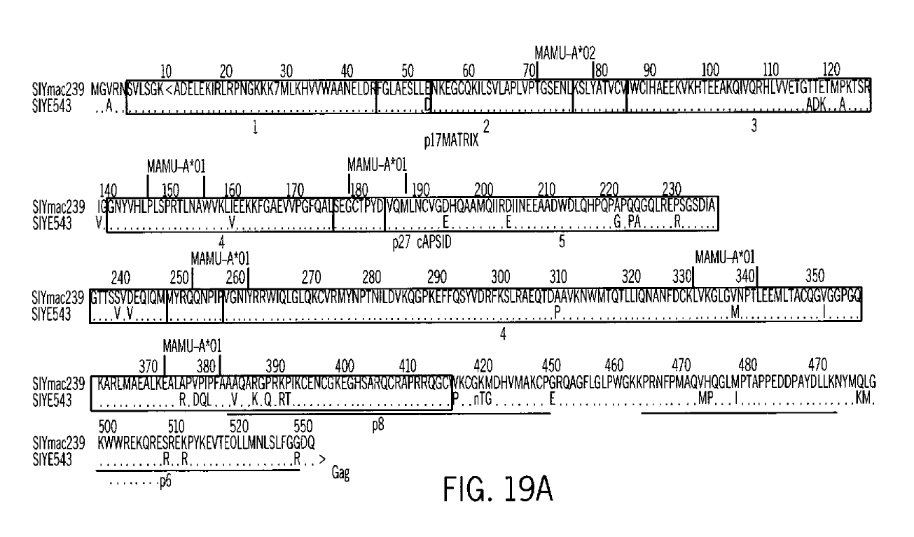

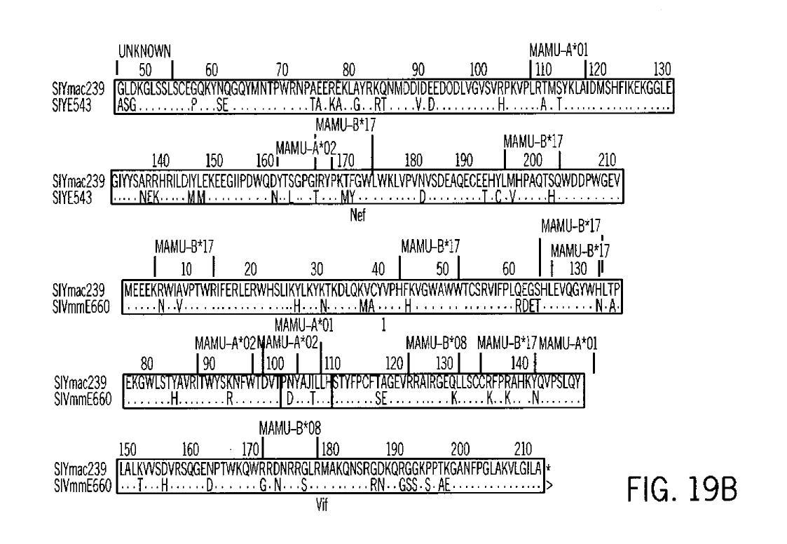

In some embodiments, suitable nucleic acids for the compositions and methods disclosed herein may include nucleic acids that encode HIV or SIV Gag, Pol, Tat, Rev, Vif, Vpr, Vpx, Vpu, or Nef polypeptide or fragments thereof. (See FIG. 19). For example, suitable nucleic acids for the compositions and methods disclosed herein may encode a polypeptide comprising the amino acid sequence of any of SEQ ID NOs:10-18. Suitable nucleic acids for the compositions and methods disclosed herein may comprise the nucleic acid sequence of any of SEQ ID NOs:1-9.

Any of the conventional vectors used for expression in eukaryotic cells may be used for directly introducing DNA into a subject. Expression vectors containing regulatory elements from eukaryotic viruses may be used in eukaryotic expression vectors (e.g., vectors containing SV40, CMV, or retroviral promoters or enhancers). Exemplary vectors include those that express proteins under the direction of such promoters as the SV40 early promoter, SV40 later promoter, metallothionein promoter, human cytomegalovirus promoter, murine mammary tumor virus promoter, and Rous sarcoma virus promoter. Therapeutic quantities of plasmid DNA can be produced for example, by fermentation in E. coli, followed by purification. Aliquots from the working cell bank are used to inoculate growth medium, and grown to saturation in shaker flasks or a bioreactor according to well known techniques. Plasmid DNA can be purified using standard bioseparation technologies such as solid phase anion-exchange resins. If required, supercoiled DNA can be isolated from the open circular and linear forms using gel electrophoresis or other methods. Purified plasmid DNA can be prepared for injection using a variety of formulations (e.g., lyophilized DNA which may be reconstituted in sterile phosphate-buffered saline (PBS)). The purified DNA may be introduced to a subject by any suitable method (e.g., intramuscular (IM) or intradermal (ID) administration).

To maximize the immunotherapeutic effects of DNA vaccines, alternative methods for formulating purified plasmid DNA may be desirable. A variety of methods have been described, and new techniques may become available. For example, cationic lipids may be used in formulating the pharmaceutical composition disclosed herein (e.g., as described by WO 93/24640; U.S. Pat. No. 5,279,833; and WO 91/06309). Protective, interactive, non-condensing compounds (PINC) may be used in formulating the pharmaceutical composition disclosed herein (e.g, glycolipids, fusogenic liposomes, and peptides).

The vectors contemplated herein may express a single HIV or SIV polypeptide or a fragment thereof. For example, the vector contemplated herein may include a polynucleotide encoding and expressing a single HIV or SIV polypeptide or a fragment thereof (e.g., a polynucleotide comprising a single HIV or SIV ORF).

The term “vector” refers to some means by which DNA fragments can be introduced into a host organism or host tissue. There are various types of vectors including plasmid, bacteriophages, cosmids, viruses, and bacteria. As used herein, a “viral vector” (e.g., an adenovirus, Sendai virus, or measles virus vector) refers to recombinant viral nucleic acid that has been engineered to express a heterologous polypeptide (e.g., an HIV polypeptide). The recombinant viral nucleic acid typically includes cis-acting elements for expression of the heterologous polypeptide. The recombinant viral nucleic acid typically is capable of being packaged into a helper virus that is capable of infecting a host cell. For example, the recombinant viral nucleic acid may include cis-acting elements for packaging. Typically, the viral vector is not replication competent or is attenuated. An “attenuated recombinant virus” refers to a virus that has been genetically altered by modern molecular biological methods (e.g., restriction endonuclease and ligase treatment, and rendered less virulent than wild type); typically by deletion of specific genes. For example, the recombinant viral nucleic acid may lack a gene essential for the efficient production or essential for the production of infectious virus.

The recombinant viral nucleic acid may function as a vector for an immunogenic retroviral protein by virtue of the recombinant viral nucleic acid encoding foreign DNA. The recombinant viral nucleic acid, packaged in a virus (e.g., a helper virus) may be introduced into a human vaccinee by standard methods for vaccination of live vaccines. A live vaccine of the invention can be administered at, for example, about 10⁴ to 10⁸ viruses/dose, or 10⁶ to 10⁹ pfu/dose. Actual dosages of such a vaccine can be readily determined by one of ordinary skill in the field of vaccine technology.

Numerous virus species can be used as the recombinant virus vectors for the pharmaceutical composition disclosed herein. A preferred recombinant virus for a viral vaccine is an adenovirus. Others include vaccinia virus, canarypox, Sendai virus, measles virus, Yellow Fever vaccine virus (e.g., 17-D or similar), retroviruses that are packaged in cells with amphotropic host range, attenuated or defective DNA viruses, such as but not limited to herpes simplex virus (HSV), papillomavirus, Epstein Barr virus (EBV), and adeno-associated virus.

### Adenovirus Vectors

The pharmaceutical compositions disclosed herein may include adenovirus vectors. Suitable adenoviral vectors may include “first generation” adenoviral vector. This group of adenoviral vectors is known in the art, and these viruses are characterized by being replication-defective. They typically have a deleted or inactivated E1 gene region, and preferably additionally have a deleted or inactivated E3 gene region. The first generation replication incompetent adenovirus vector used may be a serotype 5 adenovirus containing deletions in E1 (Ad5 base pairs 342-3523) and/or E3 (Ad5 base pairs 28133 to 30818). Those of skill in the art can easily determine the equivalent sequences for other serotypes, such as serotypes 2, 4, 12, 6, 17, 24, 33, 42, 31, 16. Adenoviral serotype 5 is preferred. However, it is envisioned that any adenovirus serotype can be used in the disclosed pharmaceutical composition, including non-human ones, as deletion of E1 genes should render all adenoviruses non-tumorogenic. The adenoviral vectors can be constructed using known techniques, such as those reviewed in Hitt et al, 1997 “Human Adenovirus Vectors for Gene Transfer into Mammalian Cells” Advances in Pharmacology 40:137-206, which is hereby incorporated by reference. In constructing the adenoviral vectors of this invention, it is often convenient to insert them in to a plasmid or shuttle vector. These techniques are known and described in Hitt et al. supra.

Viral vectors such as adenovirus vectors that express HIV and SIV polypeptides are known in the art. (See Harro C. D., et al., “Safety and Immunogenicity of Adenovirus-Vectored Near-Consensus HIV Type 1 Clade B gag Vaccines in Healthy Adults,” AIDS Res Hum Retroviruses. 2008 Dec. 24. [Epub ahead of print]; Gabitzsch E. S., et al.,” A preliminary and comparative evaluation of a novel Ad5 [E1-, E2b-] recombinant-based vaccine used to induce cell mediated immune responses,” Immunol Left. 2008 Dec. 13. [Epub ahead of print]; Ura T., et al., “Designed recombinant adenovirus type 5 vector induced envelope-specific CD8(+) cytotoxic T lymphocytes and cross-reactive neutralizing antibodies against human immunodeficiency virus type 1,” J Gene M. 2008 Dec. 8. [Epub ahead of print]; Patterson S., et al., “Use of Adenovirus in Vaccines for HIV,” Handb Exp Pharmacol: 2009;(188):275-93; Liu J., et al., “Immune control of an SIV challenge by a T-cell-based vaccine in rhesus monkeys”, Nature. 2009 Jan. 1; 457(7225):87-91. Epub 2008 Nov. 9; Ko S. Y., “Enhanced induction of intestinal cellular immunity by oral priming with enteric adenovirus 41 vectors,” J Virol. 2009 January; 83(2):748-56. Epub 2008 Nov. 5; Perreau M., “Activation of a dendritic cell-T cell axis by Ad5 immune complexes creates an improved environment for replication of HIV in T cells,” J Exp Med. 2008 Nov. 24; 205(12):2717-25. Epub 2008 Nov. 3; Sheets R. L., et al., “Biodistribution and toxicological safety of adenovirus type 5 and type 35 vectored vaccines against human immunodeficiency virus-1 (HIV-1), Ebola, or Marburg are similar despite differing adenovirus serotype vector, manufacturer's construct, or gene inserts,” J Immunotoxicol. 2008 July; 5(3):315-35; Yu S., et at, “Potent specific immune responses induced by prime-boost-boost strategies based on DNA, adenovirus, and Sendai virus vectors expressing gag gene of Chinese HIV-1 subtype B,” Vaccine. 2008 Nov. 11; 26(48):6124-31. Epub 2008 Sep. 22; Patterson L. T., et al., “Replicating adenovirus vector prime/protein boost strategies for HIV vaccine development,” Expert Opin Biol Ther. 2008 September; 8(9):1347-63. Review; Watkins D. I., et al., “Nonhuman primate models and the failure of the Merck HIV-1 vaccine in humans”, Nat Med. 2008 June; 14(6):617-21; Cox K. S., et al., “DNA gag/adenovirus type 5 (Ad5) gag and Ad5 gag/Ad5 gag vaccines induce distinct T-cell response profiles,” J Virol. 2008 August; 82(16):8161-71. Epub 2008 Jun. 4; Eller M. A., “Induction of HIV-specific functional immune responses by a multiclade HIV-1 DNA vaccine candidate in healthy Ugandans,” Vaccine. 2007 Nov. 7; 25(45):7737-42. Epub 2007 Sep. 17; and Someya K., et al., “Chimeric adenovirus type 5/35 vector encoding SIV gag and HIV env genes affords protective immunity against the simian/human immunodeficiency virus in monkeys,” Virology. 2007 Oct. 25; 367(2):390-7. Epub 2007 July 12; the contents of which are incorporated herein by reference in their entireties).

### Recombinant Attenuated Bacteria as Vectors

In some embodiments, recombinant attenuated bacteria may be utilized as vectors in the pharmaceutical compositions and vaccines disclosed herein (e.g., recombinant attenuated Shigella, Salmonella, Listeria, or Yersinia). Recombinant bacterial vaccine vectors are described in Daudel et al., “Use of attenuated bacteria as delivery vectors for DNA vaccines,” Expert Review of Vaccines, Volume 6, Number 1, February 2007, pp. 97-110(14); Shata et al., “Recent advances with recombinant bacterial vaccine vectors,” Molec. Med. Today (2000), Volume 6, Issue 2, 1 February 2000, pages 66-71; Clare & Dougan, “Live Recombinant Bacterial Vaccines,” Novel Vaccination Strategies, Apr. 16, 2004 (Editor Stefan H. E. Kaufman); Gentschev et al., “Recombinant Attenuated Bacteria for the Delivery of Subunit Vaccines,” Vaccine, Volume 19, Issues 17-19, 21 Mar. 2001, Pages 2621-2628; Garmory et al., “The use of live attenuated bacteria as a delivery system for heterologous antigens,” J. Drug Target. 2003; 11(8-10):471-9; U.S. Pat. No. 6,383,496; and U.S. Pat. No. 6,923,958 (which all are incorporated by reference herein in their entireties).

### Selection and Optimization of HIV Polypeptide-Encoding Sequences

Sequences for HIV-1 strains and many genes of HIV-1 strains are publicly available in GenBank, e.g., the sequences available under Accession Nos.: NC_{—}001802; EU293448, EU293447, EU293446, EU293445, EU293444, FJ195091, FJ195090, FJ195089, FJ195088, FJ195087, FJ195086, EU884501, EU786681, EU786680, EU786679, EU786678, EU786677, EU786676, EU786675, EU786674, EU786673, EU786672, EU786671, EU786670, EU697909, EU697908, EU697907, EU697906, EU697905, EU697904, U71182, EU69324, EU861977, FJ213783, FJ213782, FJ213781, FJ213780, AB428562, AB428561, AB428560, AB428559, AB428558, AB428557, AB428556, AB428555, AB428554, AB428553, AB428552, AB428551, DQ295195, DQ295196, DQ295194, DQ295193, DQ295192, EU446022, EU735540, EU735539, EU735538, EU735537, EU735536, EU735535, EU110097, EU110096, EU110095, EU110094, EU110093, EU110092, EU110091, EU110090, EU110089, EU110088, EU110087, EU110086, EU110085, AF193277, AF193276, AF049337, EU541617, EU220698, EF469243, DQ912823, DQ912822, EU000516, EU000515, EU000514, EU000513, EU000512, EU000511, EU000510, EU000509, EU000508, EU000507; and EU884500; which GenBank entries are incorporated herein by reference in their entireties. Primary and field isolates of HIV are available from the National Institute of Allergy and Infectious Diseases (NIAID) which has contracted with Quality Biological (Gaithersburg, Md.) to make these strains available. Strains are also available from the World Health Organization (WHO), Geneva Switzerland. In a preferred embodiment, the selected polypeptide has a consensus sequence as determined by comparing three or more strains of HIV. As disclosed herein, the polynucleotides that encode an HIV polypeptide or an SIV polypeptide may be “optimized” for expression in a human cellular environment or simian cellular environment, respectively. Thus, one aspect of this invention is the use of polynucleotides which specifically include HIV genes that are codon-optimized for expression in a human cellular environment. Optimized synthetic HIV gag genes are described in U.S. Pat. No. 6,696,291.

### Formulation of the Pharmaceutical Compositions

The pharmaceutical compositions disclosed herein may be formulated as vaccines for administration to a subject in need thereof. Such compositions can be formulated and/or administered in dosages and by techniques well known to those skilled in the medical arts taking into consideration such factors as the age, sex, weight, and condition of the particular patient, and the route of administration. The compositions may include pharmaceutical carriers, diluents, or excipients as known in the art. Further, the compositions may include preservatives (e.g., anti-microbial or anti-bacterial agents such as benzalkonium chloride) or adjuvants.

The pharmaceutical compositions may be administered prophylactically or therapeutically. In prophylactic administration, the vaccines may be administered in an amount sufficient to induce CD8⁺, CD4⁺, and/or antibody responses for protecting against infection. In therapeutic applications, the vaccines are administered to a patient in an amount sufficient to elicit a therapeutic effect (e.g., a CD8⁺, CD4⁺, and/or antibody responses to the HIV or SIV antigens or epitopes encoded by the polynucleotides of the composition, which cures or at least partially arrests or slows symptoms and/or complications of HIV or Sly infection (i.e., a “therapeutically effective dose”).

Suitable quantities of a DNA vaccine (e.g., a plasmid or naked DNA vaccine) may be about 0.1 mg to about 100 mg, preferably about 0.1 to 10 mg, but lower levels such as 0.1-2 mg or 10-100 μg can be employed. The DNA may be present at any suitable concentration (e.g., about 0.1 μg/ml to about 20 mg/ml). An HIV or SIV DNA vaccine (e.g., naked DNA or polynucleotide in an aqueous carrier) may be injected into tissue (e.g., intramuscularly, intradermally, or subcutaneously), in amounts of from 10 μl per site to about 1 ml per site. A DNA vaccine may be delivered in a physiologically compatible solution such as sterile PBS. The vaccines can also be lyophilized prior to delivery and reconstituted prior to administration.

The compositions included in the vaccine regimen of the invention can be co-administered or sequentially administered with other immunological, antigenic or vaccine or therapeutic compositions, including an adjuvant, a chemical or biological agent given in combination with or recombinantly fused to an antigen to enhance immunogenicity of the antigen. Additional therapeutic agents may include, but are not limited to interleukin-2 (IL-2) or CD40 ligand in an amount that is sufficient to further potentiate the CD8⁺ T-cell, CD4⁺ T-cell, and antibody responses. Such other compositions can also include purified antigens from the immunodeficiency virus or from the expression of such antigens by a second recombinant vector system which is able to produce additional therapeutic compositions. For example, these compositions can include a recombinant virus (e.g., an adenovirus, Sendai virus, or measles virus) or a recombinant bacteria (e.g., Shigella, Salmonella, Listeria, or Yersina bacteria) that expresses other immunodeficiency antigens or biological response modifiers (e.g., cytokines or co-stimulating molecules).

The pharmaceutical composition disclosed herein may be delivered via a variety of routes. Typical delivery routes include parenteral administration (e.g., intradermal, intramuscular or subcutaneous delivery). Other routes include oral administration, intranasal, intravaginal, intrarectal routes. The pharmaceutical composition may be formulated for intranasal or pulmonary delivery. Formulations of the pharmaceutical compositions may include liquid formulations (e.g., for oral, nasal, anal, vaginal, etc. administration, including suspensions, syrups or elixirs) and preparations for parenteral, subcutaneous, intradermal, intramuscular or intravenous administration (e.g., injectable administration) such as sterile suspensions or emulsions.

### Adjuvants

The term “adjuvant” refers to a compound or mixture that enhances the immune response to an antigen. An adjuvant can serve as a tissue depot that slowly releases the antigen and also as a lymphoid system activator that non-specifically enhances the immune response. Examples of adjuvants which may be employed include MPL-TDM adjuvant (monophosphoryl Lipid A/synthetic trehalose dicorynomycolate, e.g., available from GSK Biologics). Another suitable adjuvant is the immunostimulatory adjuvant AS021/AS02 (GSK). These immunostimulatory adjuvants are formulated to give a strong T cell response and include QS-21, a saponin from Quillay saponaria, the TL4 ligand, a monophosphoryl lipid A, together in a lipid or liposomal carrier. Other adjuvants include, but are not limited to, nonionic block co-polymer adjuvants (e.g., CRL1005), aluminum phosphates (e.g., AIPO₄), R-848 (a Th1-like adjuvant), imiquimod, PAM3CYS, poly (I:C), loxoribine, potentially useful human adjuvants such as BCG (bacille Calmette-Guerin) and Corynebacterium parvum, CpG oligodeoxynucleotides (ODN), cholera toxin derived antigens (e.g., CTA1-DD), lipopolysaccharide adjuvants, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions in water (e.g., MF59 available from Novartis Vaccines or Montanide ISA 720), keyhole limpet hemocyanins, and dinitrophenol.

Additional Prophylactic and/or Therapeutic Agents

The pharmaceutical compositions disclosed herein may further or additionally comprise at least one antiviral chemotherapeutic compound. Non-limiting examples can be selected from at least one of the group consisting of gamma globulin, amantadine, guanidine, hydroxy benzimidazole, interferon-α, interferon-β, interferon-γ, interleukin-16, thiosemicarbarzones, methisazone, rifampin, ribavirin, pyrimidine analogs (e.g., AZT and/or 3TC), purine analogs, foscarnet, phosphonoacetic acid, acyclovir, dideoxynucleosides, protease inhibitors (e.g., saquinavir; indinavir; ritonavir; AG 1343; and VX-2/78), chemokines, such as RANTES, MIP1α or MTP1β or ganciclovir.

### Prime-Boost Vaccination Regimen

As used herein, a “prime-boost vaccination regimen” refers to a regimen in which a subject is administered a first composition one or more times (e.g., two or three times with about 2, 3, or 4 weeks between administrations) and then after a determined period of time (e.g., about 2 weeks, about 4 weeks, about 2 months, about 3 months, about 4 months, about 5 months, about 6 months, or longer), the subject is administered a second composition. The second composition may also be administered more than once, with at least 2, 3, or 4 weeks between administrations. The first and second compositions may be the same or different. For example, the first composition may include a naked DNA vaccine and the second composition may include a viral vector vaccine (e.g., a recombinant adenovirus, vaccinia virus, Sendai virus, or measles virus vaccine). In another example, the first composition may include a first naked DNA vaccine and the second composition may include a second naked DNA vaccine. In another example, the first composition may include a first recombinant virus vector and the second composition may include a second recombinant virus vector.

Where the first or second pharmaceutical compositions include one or more recombinant virus vectors (e.g., recombinant adenovirus, Sendai virus, or measles virus vectors), the vectors may be administered alone, or may be part of a prime and boost administration regimen. For example, the disclosed methods may include priming a subject with a plasmid vaccine by administering the plasmid vaccine at least one time, allowing a predetermined length of time to pass, and then boosting by administering another vaccine (e.g., an adenovirus, Sendai viral, measles virus, or recombinant attenuated bacterial vaccine). Multiple primings (typically 1-4) can be employed, although more may be used. The length of time between priming and boost may typically vary from about four weeks to a year, but other time frames may be used. In experiments with rhesus monkeys, the animals may be primed three or four times with plasmid vaccines, then boosted about 4 months later with another vaccine (e.g., an adenovirus, Sendai viral, measles virus, or recombinant attenuated bacterial vaccine).

The use of a priming regimen may be particularly preferred in situations where a person has a pre-existing anti-adenovirus immune response. Prime-boost vaccination regimens are described in U.S. Pat. Nos. 6,723,558; 6,787,351; and 7,094,408.

### Characterization of the Immune Response in Vaccinated Individuals

The pharmaceutical compositions disclosed herein may be delivered to subjects at risk for infection with HIV or SIV or to subjects who are infected with HIV or SIV. In order to assess the efficacy of the vaccine, the immune response can be assessed by measuring the induction of CD8⁺ responses and antibodies to particular epitopes. CD8⁺ T-cell responses may be measured, for example, by using tetramer staining of fresh or cultured PBMC, ELISPOT assays or by using functional cytotoxicity assays, which are well-known to those of skill in the art and are described herein. Antibody responses may be measured by assays known in the art such as ELISA.

Viral titer or load and CDC T cell counts after immunization can be measured in subjects who are already infected. For example, subjects may demonstrate a decrease in viral load and an increase in CD4⁺ cell counts after immunization (e.g., an increase in CD4⁺ cell counts of at least about 2×, 3×, or 4× relative to the level prior to immunization).

All publications and patent applications cited in this specification are herein incorporated by reference as if each individual publication or patent application were specifically and individually indicated to be incorporated by reference.

### EXAMPLES

The following examples are illustrative and are not intended to limit the disclosed subject matter.

### Example 1

Reference is made to Wilson et al., “Vaccine-induced Cellular Responses Control SIV Replication After Heterologous Challenge,” J. Virol. doi:10.1128/JVI.00272-09, published online ahead of print on 29 Apr. 2009, the content of which is incorporated by reference herein it its entirety.

### Summary

All HIV vaccine efficacy trials to date have ended in failure. Structural features of the Env glycoprotein and its enormous variability have frustrated efforts to induce broadly-reactive neutralizing antibodies. To explore the extent to which vaccine-induced cellular immune responses, in the absence of neutralizing antibodies, can control replication of a heterologous, mucosal viral challenge, eight macaques were vaccinated with a DNA/Ad5 regimen expressing all of the proteins of SIVmac239 except Env. Vaccinees mounted high-frequency T cell responses against 11-34 epitopes. The vaccinees and eight naïve animals were challenged with the heterologous biological isolate SIVsmE660, using a regimen intended to mimic typical human HIV exposures resulting in infection. Viral loads in the vaccinees were significantly less at both peak (1.9 log reduction p<0.03) and at set point (3.3 log reduction p<0.003) than those of control naïve animals. Five of eight vaccinated macaques controlled acute peak viral replication to less than 80,000 vRNA copy Eq/ml and to less than 100 vRNA copy Eq/ml in the chronic phase. These results demonstrate that broad vaccine-induced cellular immune responses can effectively control replication of a pathogenic, heterologous AIDS virus, suggesting that T cell based vaccines may have greater potential than previously appreciated.

### Introduction

It has been impossible thus far for vaccines to engender broadly reactive neutralizing antibodies against HIV [12, 54]. Investigators have therefore focused their attention on T cell-based vaccines [9, 18, 26, 30, 34, 39, 48, 55]. Previous pre-clinical studies in nonhuman primates have shown that vaccine-induced T cell responses can partially control replication of homologous challenge viruses in the chronic phase [34, 56]. Unfortunately, however, Sly virus loads exceeded one million copies in almost every vaccinated animal during the acute phase. Given the high levels of viral replication observed in these vaccinated macaques, it is possible that such T cell-based vaccines might not be able to reduce transmission during the acute phase of infection in humans. These high levels of replication during the acute phase likely resulted in the generation of diverse viral quasispecies, providing the substrate for immune selection and eventual escape. Furthermore, in these studies vaccinated animals were challenged with viruses that were similar to the SIV sequences in the vaccine constructs. Given the diversity of HIV, human vaccinees will never be exposed to viruses with a comparable level of sequence similarity to the vaccine constructs.

An HIV-1 vaccine that induced T cell responses exclusively has recently failed to show efficacy against the incidence of HIV infection and viremia in clinical testing. The STEP trial of a recombinant Adenovirus 5 (rAd5)-vectored vaccine designed to induce HIV-specific T cell responses in humans was widely seen as an important test of the T cell vaccine concept (see HIV Vaccine Trials Network (HVTN) website) [11, 42]. The lack of vaccine efficacy in the STEP trial has led some to conclude that T cell-based vaccines may not be a viable approach to solve the AIDS epidemic [1, 49, 59]. However, STEP trial vaccinees who became infected recognized a median of only five epitopes, mostly in the conserved proteins Gag and Pol. Given the sequence diversity of HIV [19], several of these vaccine-elicited T cell responses may not have recognized epitopes in the infecting virus, and therefore not constituted an adequate test of the T cell vaccine concept. Therefore, a much broader vaccine-induced T cell response against an AIDS virus was tested to determine whether it could more effectively impact viral replication after challenge with a heterologous virus in macaques.

### Methods

Animals and viruses. The animals in this study were Indian rhesus macaques (Macaca mulatta) from the Wisconsin National Primate Research Center colony. They were typed for major histocompatibility complex (MHC) class I alleles Mamu-A*01, Mamu-A*02, Mamu-A*08, Mamu-A*11, Mamu-B* 01, Matnu-B*03, Mamu-B*04, Mamu-B*08, Mamu-B*17, and Mamu-B*29 by sequence-specific PCR [28, 36]. Animals that were Mamu-A*02 positive were chosen for the study, but animals positive for Mamu-A*01, Mamu-B*08 or Mamu-B*17 were excluded. It has been observed that the presence of either the Mamu-B*08 or Mamu-B*17 allele alone is correlated with a reduction in plasma viremia [36, 60]. The animals were cared for according to the regulations and guidelines of the University of Wisconsin Institutional Animal Care and Use Committee.

Vaccination. Genes coding for SIVmac239 Gag, Tat, Rev, Nef, Pol, Vif, Vpr and Vpx were synthesized based on codons frequently used in mammalian cells [51] and cloned into the VIR vector. Five mg of plasmid DNA formulated with 7.5 mg of CRL1005 mixed with benzalkonium chloride was injected i.m. in a volume of 0.5 ml. Before the intramuscular injection, the formulation was warmed slowly to room temperature from a frozen stock. DNA was injected at 6 different sites, left and right triceps, left and right quadriceps, left and right gastrocnemius. All plasmids were injected at a unique site, with the exception of Vif, Vpr and Vpx. Although these were formulated on separate plasmids, all three injections were given at the same site. Sites to which each particular plasmid was administered rotated every injection (including Ad5 boost) to ensure that no site received a vector encoding the same protein twice. For instance, if the vector encoding gag was injected into the right quadriceps for the first DNA prime, on the next prime, this vector would be injected into a different site, perhaps the left quadriceps, then on the third prime into yet a different site, such as the right triceps, etc. In this way, the draining lymph nodes are exposed to different encoded proteins each time and are not re-boosted with the same protein sequence, in order to avoid potentially inducing immunodominance. The same codon-optimized genes were cloned into an adenoviral vector based on serotype 5 adenovirus that had been rendered incompetent to replicate by deletions of the E1 viral gene and subsequently propagated in E1-expressing PER.C6 cells as previously described [51]. Six different Ad5 vectors were created, five contained the single coding sequences for Gag, Tat, Rev, Nef and Pol, while the sixth encoded Vif, Vpr and Vpx, all very small proteins. Viral particles (10¹¹) of Ad5 were delivered i.m. in a volume of 0.5 mls to the same six sites as previously used for DNA priming, again rotating the site to which each protein sequence was administered.

Challenge. Thirty-seven weeks after vaccination was completed, the vaccinees and 8 naïve control animals were challenged intrarectally with SIVsmE660 (800 TCID₅₀, 1.2×10⁷ SIV RNA copy Eq). A repeated mucosal challenge regimen [40] with SIVsmE660 was used to more closely mimic. HIV transmission in humans. Animals were initially challenged with this dose every three weeks, up to 5 times. Animals were considered to be SIV positive after at least two subsequent positive viral load determinations and were no longer challenged after it was determined that they were positive. After 5 such challenges, animals that were still SIV negative were challenged intrarectally every other week with a dose of 4000 TCID₅₀. It took up to 6 additional challenges before all animals were infected.

Viral load determination. Levels of circulating plasma virus were determined using a previously described QRT-PCR assay [45]. Virus concentrations were determined by interpolation onto a standard curve of in vitro-transcribed RNA standards in serial 10-fold dilutions using the LIGHTCYCLER™ 2.0 (software version 4; Roche).

IFN-γ ELISPOT assays. Fresh PBMC isolated from EDTA anticoagulated blood were used in ELISPOT assays for the detection of IFN-γ—secreting cells as previously described [35]. As in previous publications, to determine positivity, the number of spots in duplicate wells (100,000 cells per well) was averaged, and the background subtracted. This resultant number is required to be greater than 5 spots (50 spot forming cells (SFC)/million PBMC) and also greater than 2 standard deviations over background. Positive results are multiplied by 10 to get SFCs/million cells. Some of the peptides used in these assays were obtained through the AIDS Research and Reference reagent program, Division of AIDS, National Institute of Allergy and Infectious Disease, and National Institutes of Health. Additionally, responses by CD8− cells were examined by depleting PBMC of CD8+ cells using a CD8 Microbead kit for nonhuman primates (Miltenyi Biotec) according to the manufacturer's instructions.

T cell quantitation. CD8+ and CD4+ subsets and memory populations were monitored by staining PBMC with fluorescently labeled antibodies specific for CD3 Alexa 700 (BD Pharmingen, San Diego, Calif.), CD4 PerCP (Miltenyi, Auburn, Calif.), CD8 Pacific Blue (BD Pharmingen), CD95 FITC (BD Pharmingen), CD28 PE (Becton Dickinson, San Jose, Calif.), beta7 integrin APC (Becton Dickinson). In brief, 500,000 PBMC were incubated with these antibodies for 30 min at room temperature. The samples were then washed twice, fixed with paraformaldehyde, and run on a BD-LSR-B flow cytometer (Becton Dickinson) using FACSDiva software (BD Biosciences). Data were analyzed using FlowJo software. Absolute counts were calculated by multiplying the frequency of CD3+/CD4+ T cells or CD3+/CD4+/CD95+CD28+ T cells (central memory) or CD3+/CD4+/CD95+CD28+ T cells (effector memory) within the lymphocyte gate by the lymphocyte counts per microliter of blood obtained from matching complete blood counts.

Viral RNA extraction and cDNA synthesis. From each plasma specimen, approximately 20,000 viral RNA copies were extracted using the QIAamp Viral RNA Mini Kit (Qiagen, Valencia, Calif.). RNA was eluted and immediately subjected to cDNA synthesis. Reverse transcription of RNA to single stranded cDNA was performed using SuperScript III reverse transcriptase according to manufacturer's recommendations (Invitrogen Life Technologies, Carlsbad, Calif.). Briefly, each cDNA reaction included 1× RT buffer, 0.5 mM of each deoxynucleoside triphosphate, 5 mM dithiothreitol, 2 units/ml RNaseOUT (RNase inhibitor), 10 units/ml of SuperScript III reverse transcriptase, and 0.25 mM antisense primer SIVsm/macEnvR1 5′-TGTAATAAATCCCTTCCAGTCCCCCC-3′ (nt 9454-9479 in Mac239) (SEQ ID NO:19). The mixture was incubated at 50° C. for 60 minutes followed by an increase in temperature to 55° C. for an additional 60 minutes. The reaction was then heat-inactivated at 70° C. for 15 minutes and treated with 2 units of RNase H at 37° C. for 20 minutes. The newly synthesized cDNA was used immediately or frozen at −80° C.

Single genome amplification (SGA). cDNA was serially diluted and distributed among wells of replicate 96-well plates so as to identify a dilution where PCR positive wells constituted less than 30% of the total number of reactions, as previously described [29, 47]. At this dilution, most wells contain amplicons derived from a single cDNA molecule. This was confirmed in every positive well by direct sequencing of the amplicon and inspection of the sequence for mixed bases (double peaks), which would be indicative of priming from more than one original template or the introduction of PCR error in early cycles. Any sequence with mixed bases was excluded from further analysis. PCR amplification was carried out in the presence of 1× High Fidelity Platinum PCR buffer, 2 mM MgSO₄, 0.2 mM of each deoxynucleoside triphosphate, 0.2 μM of each primer, and 0.025 units/μl Platinum Taq High Fidelity polymerase in a 20-μl reaction (Invitrogen, Carlsbad, Calif.). First round PCR primers included sense primer SIVsm/macEnvF1 5′-CCTCCCCCTCCAGGACTAGC-3′ (nt 6127-6146 in SIVmac239) (SEQ ID NO:20) and antisense primer SIVsm/macEnvR1 5′-TGTAATAAATCCCTTCCAGTCCCCCC-3 (nt 9454-9479 in SIVmac239) (SEQ ID NO:19), which generated a ˜3.3 kb amplicon. PCR was performed in MicroAmp 96-well reaction plates (Applied Biosystems, Foster City, Calif.) with the following PCR parameters: 1 cycle of 94° C. for 2 min; 35 cycles of a denaturing step of 94° C. for 15 s, an annealing step of 55° C. for 30 s, an extension step of 68° C. for 4 min, followed by a final extension of 68° C. for 10 min. Next, 2 μl from first round PCR product was added to a second round PCR reaction that included the sense primer SIVsmEnvF2 5′-TATGATAGACATGGAGACACCCTTGAAGGAGC-3′ (nt 6292-6323 in SIVmac239) (SEQ ID NO:21) or SIVmacEnvF2 5′-TATAATAGACATGGAGACACCCTTGAGGGAGC-3′ (nt 6292-6323 in Mac239) (SEQ ID NO:21) and antisense primer SIVsmEnvR2 5′-ATGAGACATRTCTATTGCCAATTTGTA-3′ (nt 9413-9439 in SIVmac239) (SEQ ID NO:22). The second round PCR reaction was carried out under the same conditions used for first round PCR but with a total of 45 cycles. Amplicons were inspected on precast 1% agarose E-gels 96 (Invitrogen Life Technologies, Carlsbad, Calif.). All PCR procedures were carried out under PCR clean room conditions using procedural safeguards against sample contamination, including prc-aliquoting of all reagents, use of dedicated equipment, and physical separation of sample processing from pre- and post-PCR amplification steps.

DNA sequencing. Env gene amplicons were directly sequenced by cycle-sequencing using BigDye terminator chemistry and protocols recommended by the manufacturer (Applied Biosystems; Foster City, Calif.). Sequencing reaction products were analyzed with an ABI 3730x1 genetic analyzer (Applied Biosystems; Foster City, Calif.): Both DNA strands were sequenced using partially overlapping fragments. Individual sequence fragments for each amplicon were assembled and edited using the Sequencher program 4.7 (Gene Codes; Ann Arbor, Mich.). Inspection of individual chromatograms allowed for the confirmation of amplicons derived from single versus multiple templates. The absence of mixed bases at each nucleotide position throughout the entire env gene was taken as evidence of single genome amplification from a single vRNA/cDNA template. This quality control measure enabled us to exclude from the analysis amplicons that resulted from PCR-generated in vitro recombination events or Taq polymerase errors and to obtain multiple individual env sequences that proportionately represented those circulating in vivo.

Sequence alignments and phylogenetic analysis. All alignments and phylogenetic trees were made with Clustal W [52] Alignments are shown in Highlighter, which is a sequence analytical tool available from the Los Alamos National Laboratory (LANL) HIV database website that displays the location and identity of nucleotide substitutions in a visually informative manner and allows tracing of common ancestry between sequences based on individual nucleotide polymorphisms. All 429 env sequences from ramp-up and peak viremia and 32 env sequences from the inoculum stock were deposited in GenBank (Accession numbers FJ847845-FJ847927).

Hypermutated samples. Enrichment for mutations with APOBEC signatures was assessed by visual inspection of the sequences and confirmed using Hypermut 2.0 available from the Los Alamos National Laboratory (LANL) HIV database website.

Statistical Analyses. Plasma viral concentrations were log-transformed to reduce right-skewness and heteroscedasticity. Peak and average viral loads were computed for each animal across days 28 through 140, with mostly complete data present through day 112. P-values were computed using two different statistical tests which compare controls to vaccinees. The Welch two sample t-test tests the null hypothesis of equal means against the two-sided alternative hypothesis, without assuming equal variance. The permutation test is a non-parametric distribution-free test of identical distributions across the two groups. Two-tailed P-values under 0.05 are highlighted with a caret.

### Results

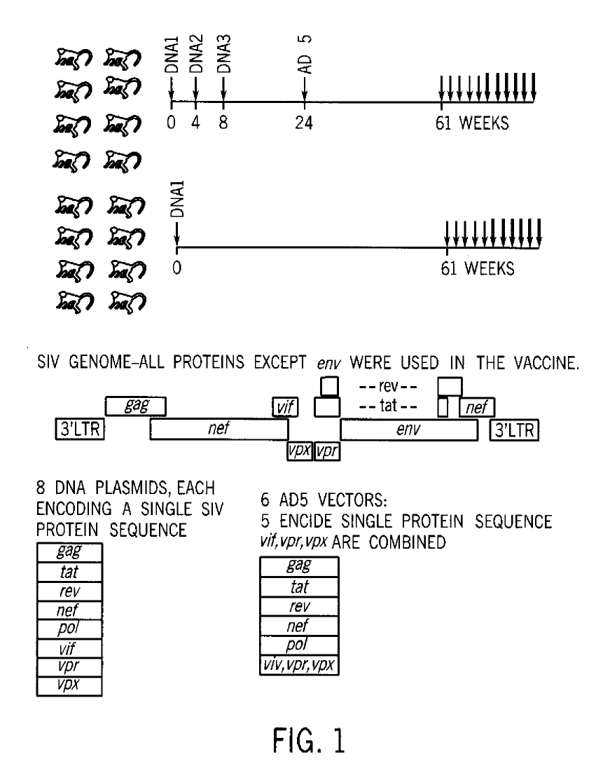

DNA prime/Adenovirus 5 boost vaccination using SIVmac239 sequences. Eight Indian rhesus macaques were vaccinated with a DNA prime, Adenovirus 5 (Ad5) boost regimen encoding all of the SIVmac239 proteins except Env (FIG. 1). Any animals expressing the MHC class I alleles Mamu-A*01, -B*08, or -B*17 that have been implicated in control of viral replication [36] were excluded. All of these eight vaccinees and an additional eight naïve control macaques expressed Mamu-A*02 so that CD8⁺ T-cell responses could be followed against a variety of well-defined CD8⁺ T-cell epitopes [37] (Table 7). Vaccine-induced T cell responses were measured by performing whole proteome Elispot with whole PBMC at day 14 after the Ad5 boost. Eighty-three pools each consisting of 10 15-mers (overlapping by 11 amino acids) spanning the nine SIV open reading frames were used. Subsequently, responses were mapped using as many responses as possible (depending on blood availability) down to single 15-mer peptides in the ensuing weeks. CD4+ and CD8+ T cell responses could not be distinguished during the vaccination stage. The vaccine induced high frequency T cell responses against 11-34 epitopes (Table 1).

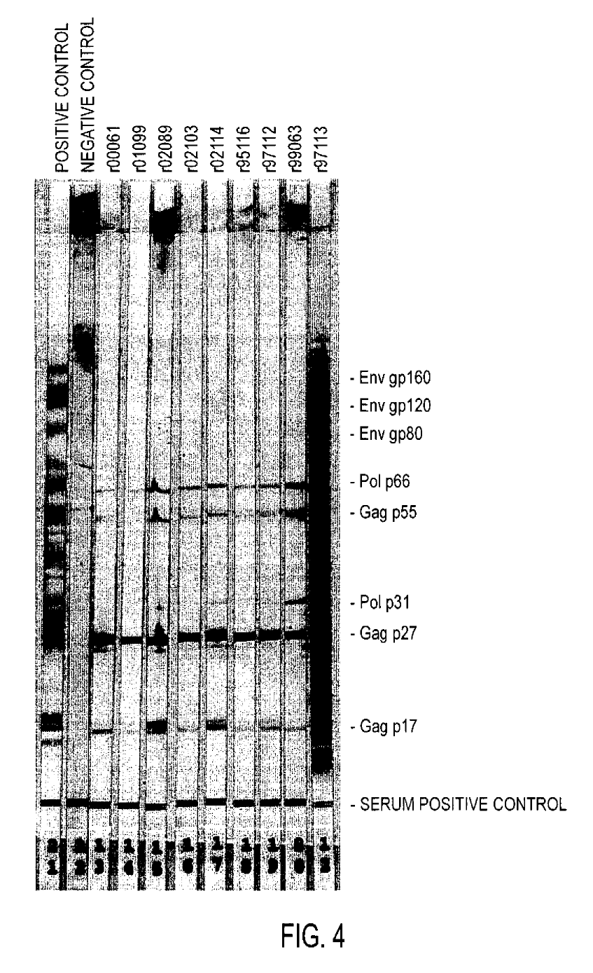

Interestingly, all vaccinated macaques made strong CD8+ T-cell responses against the Mamu-A*02-restricted epitope Env RY8(788-795) even though Env was not present in the vaccine. Sequencing of all vaccine constructs revealed no env sequences and Western blots were negative for antibody against Env (FIG. 4). Since the Envelope protein itself was not encoded by any of the vaccine vectors, the Env RY8 epitope may have been derived from translation of an alternate reading frame of the Rev-encoding plasmid and Ad5 vector. To test this hypothesis directly, Mamu-A *02-expressing 221 cells were transfected with the Rev or the Gag plasmid or an Env-expressing plasmid not included in the vaccine. Twenty-four hours later, whether these cells could present the RY8 epitope to a RY8-specific CTL line was tested. The RY8-specific CTL line recognized the Rev- and Env-transfected cells but not the Gag-transfected cells, indicating that the Rev plasmid was the source of the RY8 epitope and likely the RY8-directed response in the vaccinated animals (data not shown). The sequence of the Rev plasmid does not contain an alternate methionine start codon upstream of the RY8 epitope. However, translation of the epitope could be due to ribosomal slippage or translation initiation at a non-Met start codon. In SIVsmE660, there are four substitutions in this Env RY8 (Table 7) and vaccine-induced T-cell responses against this epitope were not expanded after challenge. In one animal, r02114, there was an additional response to an Env peptide in the N-terminal region of the protein, mapped to a 15-mer, NATIPLFCATKNRDT, Env(37-51) (SEQ ID NO:23). This response was sustained well into the vaccine phase, but was not expanded after challenge. Thus, it is unlikely that these Env-specific CD8 T cell responses affected the outcome after challenge.

Titration of heterologous challenge virus to mimic human HIV exposure. To date, the majority of non-human primate vaccine trials have used challenge viruses homologous to the vaccine, and many of those have involved mucosal challenges that have been carried out using a high dose inoculum of challenge virus to ensure that all control naïve animals become infected. By contrast, the majority of vaccinated humans will likely be exposed to multiple lower doses of a virus that is significantly different from the vaccine. Furthermore this exposure will take place across a mucosal surface. Therefore, to model human exposure to HIV, a repeated rectal challenge in macaques using a swarm virus (SIVsmE660 [10, 20, 25]) was developed. This virus is considerably different from the SIVmac239 viral sequences in the vaccine, and a dose that mimics the biology of human HIV infection was utilized. HIV isolates within a single clade typically vary by as much as 10-20%, depending on the gene of reference [19]. Vaccinated humans will likely encounter viruses that differ by at least this much from the vaccine strain. The sequence of SIVsmE660 differs from the SIVmac239-derived vaccine sequences by approximately 15% of its amino acids (Table 2).

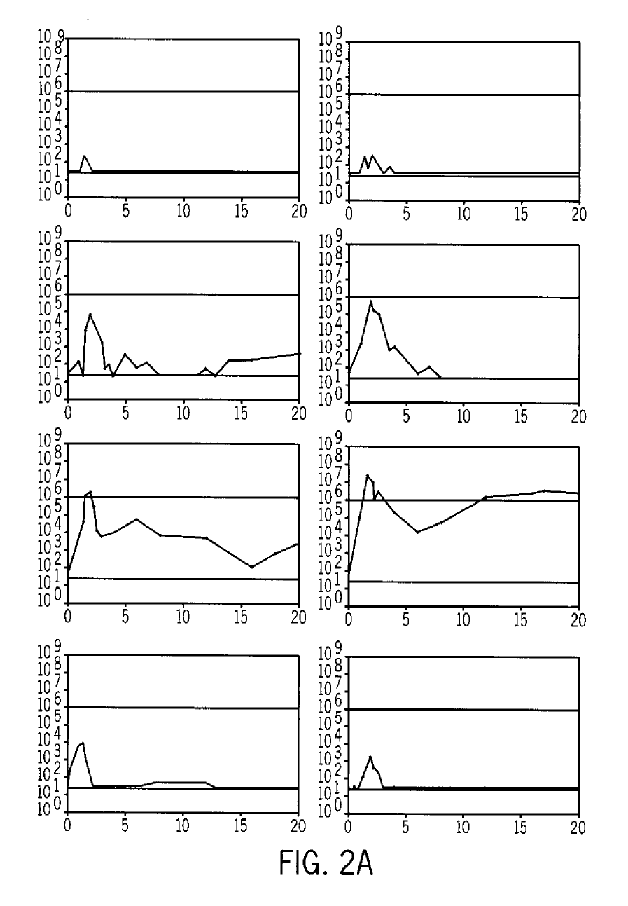

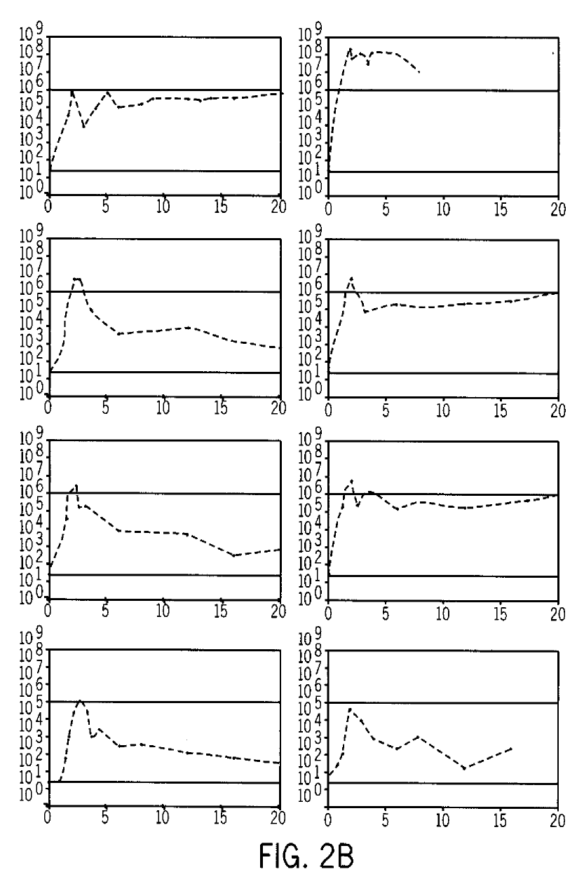

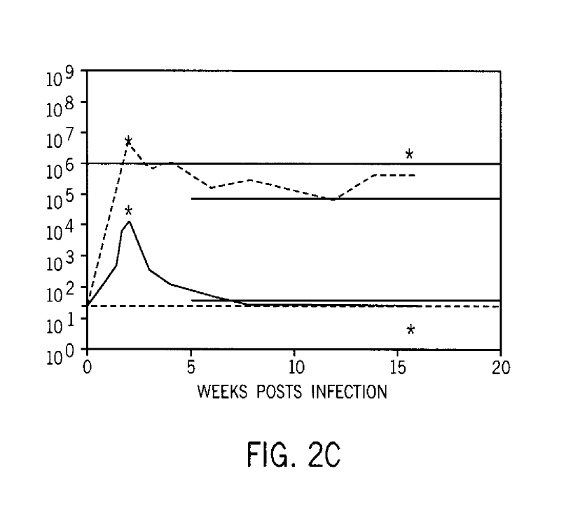

Recent reports indicate that in acutely HIV infected individuals only one or a few virus variants are involved in establishing the initial systemic infection [29]. To recapitulate this scenario, the pathogenic SIVsmE660 stock [45] was titered using two different doses to define a challenge inoculum at which only one or a few viral variants are involved in establishing a disseminated systemic infection during the acute phase. A single rectal challenge with 4,000 TCID₅₀ (233 μl of tissue culture fluid at 2.58×10⁸ vRNA copy Eq/ml, for a total of 6×10⁷ vRNA copy Eq) infected two of two macaques (FIG. 2A). A single rectal challenge with one-tenth of this initial dose, 400 TCID₅₀, (23 μl of tissue culture fluid at 2.58×10⁸ vRNA copy Eq/ml for a total of 6×10⁶ vRNA copy Eq) infected only one of two macaques. After a second challenge at the lower dose, the second animal became infected. 207 full-length SIV env genes from plasma virion RNA (vRNA) from the 4 animals were amplified and sequenced (median of 52 sequences per animal; range 19-73). Single genome amplification (SGA) [29] of plasma vRNA from acute phase plasma revealed that a minimum of 3-10 viral variants had been transmitted to the two animals that received the single dose of 4,000 TCID₅₀ (data not shown). Productive infection by only 1 viral variant was evident in each of the two animals infected at the lower dose of 400 TCID50 (data not shown). Similar findings in a recent titration study of 18 Indian rhesus macaques infected intrarectally or intravenously with SIVsmE660 or SIVmac251 were made (B. F. K. et al., Low Dose Rectal Inoculation of Rhesus Macaques by SIVsmE660 or SIVmac251 Recapitulates Human Mucosal Infection by HIV-1. J Exp Med, 2009 May 11; 206(5):1117-34. Epub 2009 May 4). Thus, a challenge dose of 800 TCID₅₀ of the heterologous swarm virus SIVsmE660 initially was elected to mimic mucosal HIV exposure in humans.

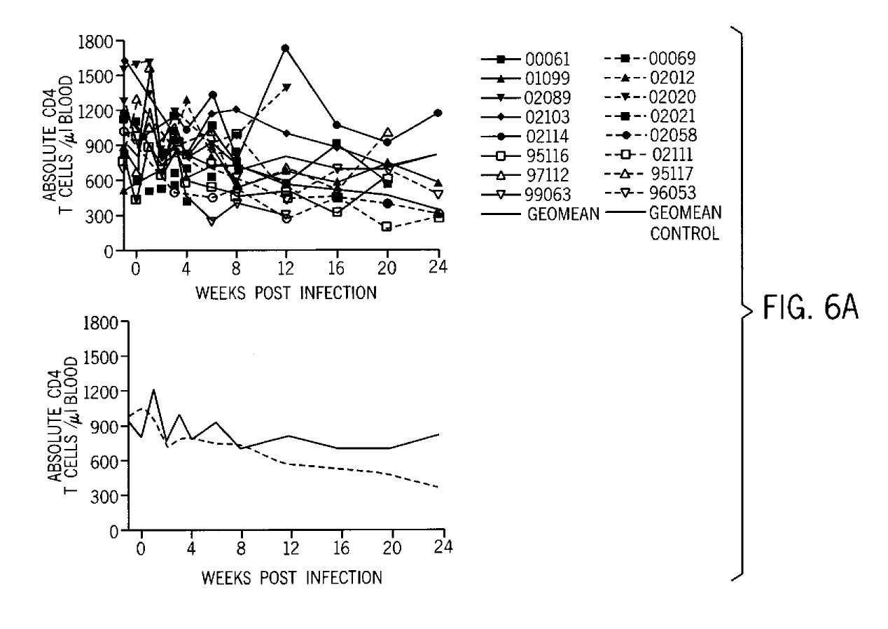

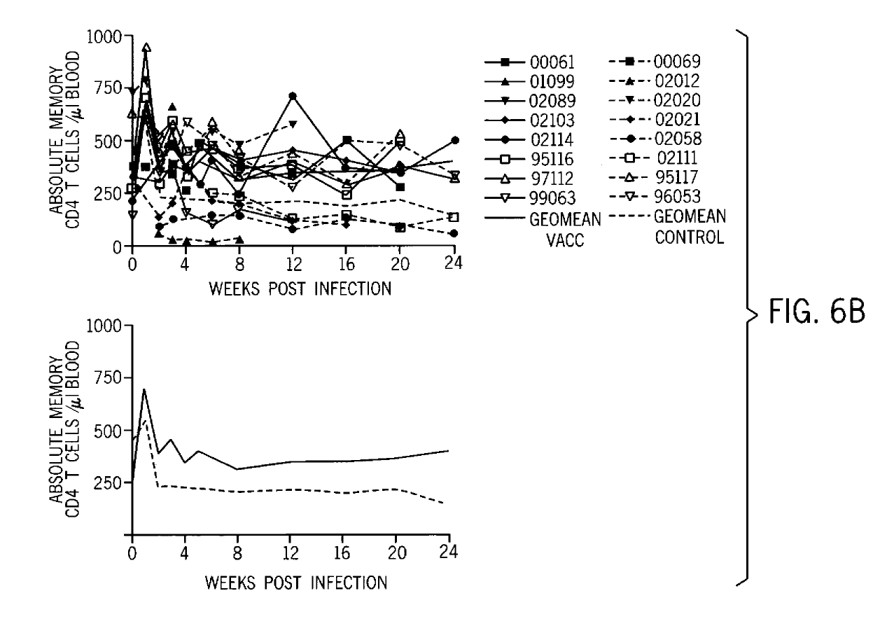

Challenge with SIVsmE660. Vaccinees (and 8 naïve control animals) were challenged mucosally 37 weeks after the Ad5 boost with repeated doses (800 TCID₅₀) of the heterologous swarm virus SIVsmE660 every three weeks for as many as five inoculations. At 33 weeks after the Ad5 boost, the vaccine-induced T cell responses had diminished considerably, especially the CD4+ T cell responses (Table 8). Viral loads were assessed at days 7, 9, and 11 after each challenge and if two of these were positive, the animal was not re-challenged. Five of eight vaccinees and six of eight naïve controls became infected after these challenges. Surprisingly, two of the vaccinees (r00061 and r02103) had peak viral loads of only 300 vRNA copy Eq/ml (FIG. 2A). Another vaccinee (r02114) had an acute phase peak of only 71,000 vRNA copy Eq/ml. These three vaccinees have plasma viral loads that are either undetectable or less than 100 vRNA copy Eq/ml in the chronic phase. Additionally, a fourth vaccinee (r01099) with a peak viremia of 456,000 vRNA copy Eq/ml now has undetectable plasma viremia at 20 weeks post infection. While none of the vaccinees have experienced a loss of either total or memory CD4+ T cell counts after challenge, three of six infected control animals have reduced total memory CD4+ T cell counts during the acute phase (FIG. 6). However, the differences between these groups were not significant. CD4+ T cells in the BAL also were analyzed. While the percent of CD4+ T cells is higher in vaccinees than in control animals, the difference is not significant (data not shown). Absolute counts of T cells in the BAL were not obtained.

The three remaining uninfected vaccines then were challenged, along with the two uninfected naïve control animals with up to six repeated challenges of an increased dose of SIVsmE660 (4,000 TCID₅₀). All five animals became infected. Remarkably, two of the three vaccinees (r97112 and r02089) controlled virus replication to less than 8,000 vRNA copies/ml during the acute phase (FIG. 2A). Interestingly, these two animals were the ones that mounted the broadest and highest frequency immune responses after vaccination (Table 1).

Even though there was no difference between the number of challenges required to cause infection in the vaccinees and controls (Table 3), the vaccinees exhibited significantly reduced viral replication in both the acute and chronic phases of infection. Peak plasma SIV RNA levels were 3.2×10⁴ vRNA copy Eq/ml for vaccinees versus 2.5×10⁶ vRNA copy Eq/ml for controls (p<0.0263) (FIG. 2C, Table 4). At 16 weeks post-infection, vaccinated animals had a mean viral load of 7.9 vRNA copy Eq/ml compared with control animals of 3.2×10⁴ vRNA copy Eq/ml (P<0.0033).

Identification and enumeration of transmitted viruses. SGA-direct amplicon sequencing was used to identify and estimate the numbers of viral variants involved in establishing disseminated systemic acute infection in control and vaccinated animals. 222 full-length SIV env genes from plasma vRNA from 10 animals (6 controls and 4 vaccinees) one to two weeks after infection were amplified and sequenced (median of 22 sequences per animal; range 10-45). Both control and vaccinated animals were infected by 1-4 viruses (control—median of 3; vaccinated—median of 1.5) (Table 5). Although there was a trend towards lower numbers of transmitted variants in vaccinated animals compared with control animals, this difference was not statistically significant. Interestingly, some animals productively infected by single viruses had a large proportion of viral sequences with APOBEC mediated G-to-A hypermutation (data not shown). Enrichment for G-to-A mutations was observed in titration animals, control animals, and vaccinated animals (data not shown).

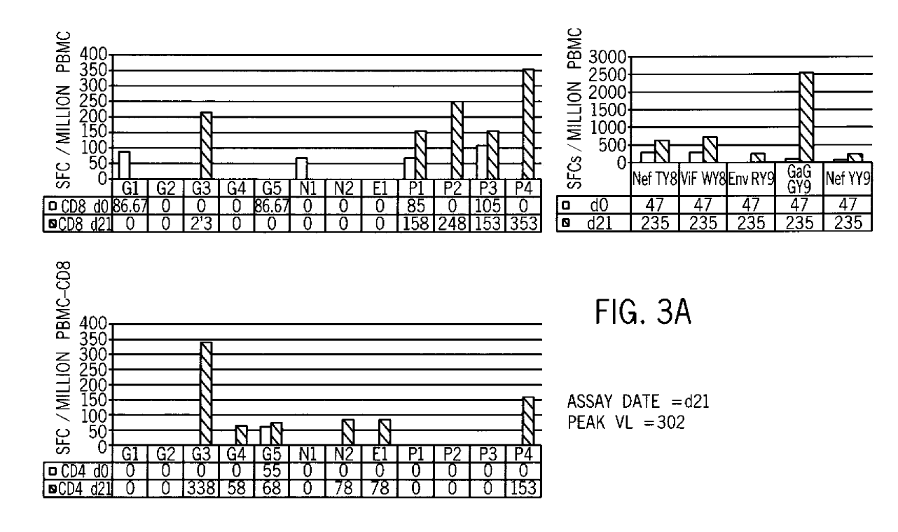

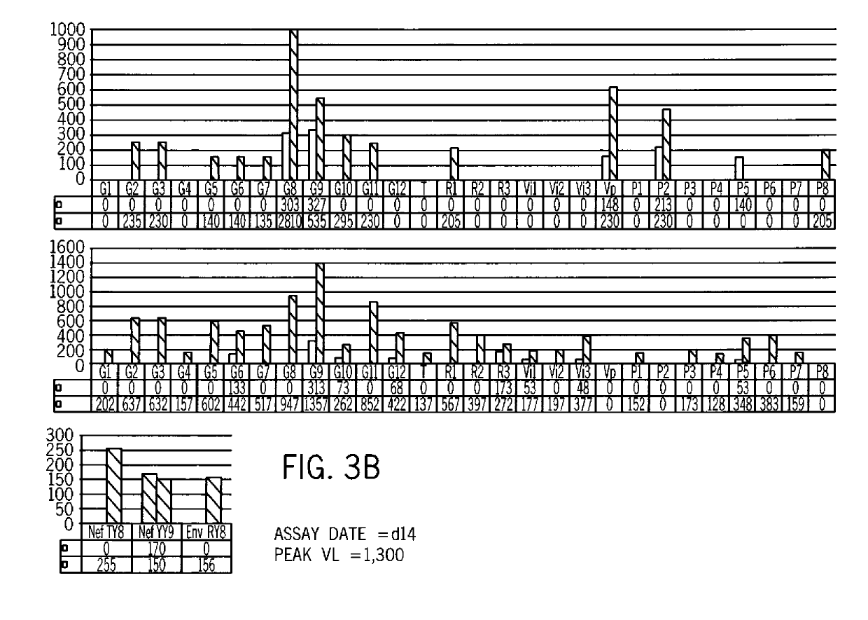

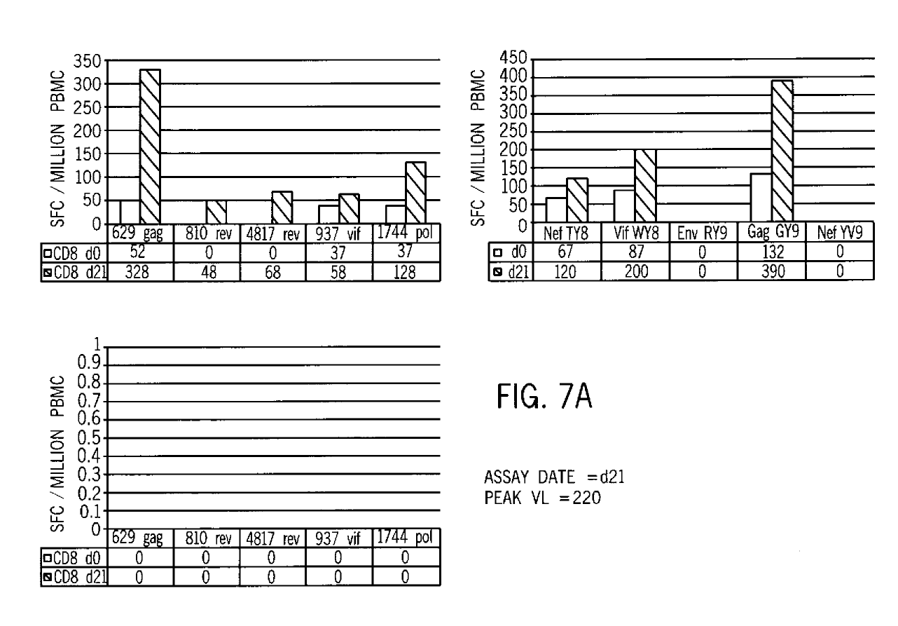

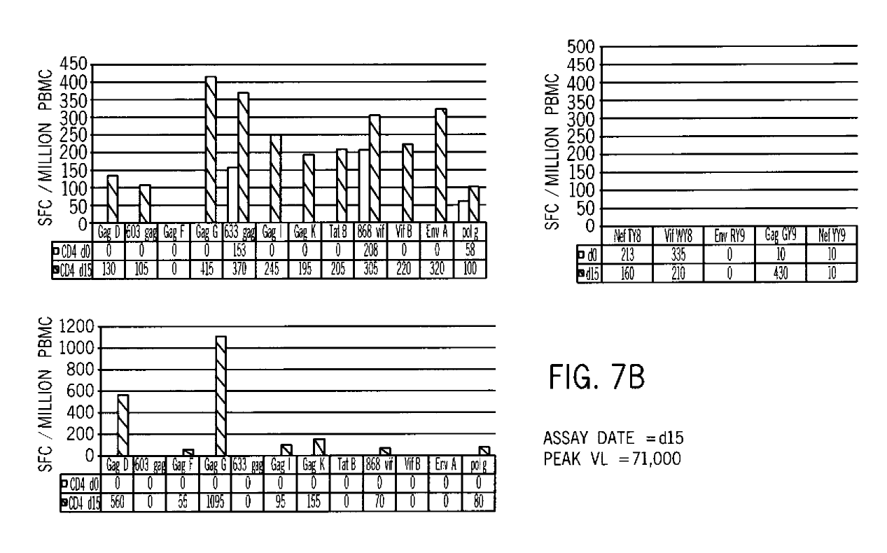

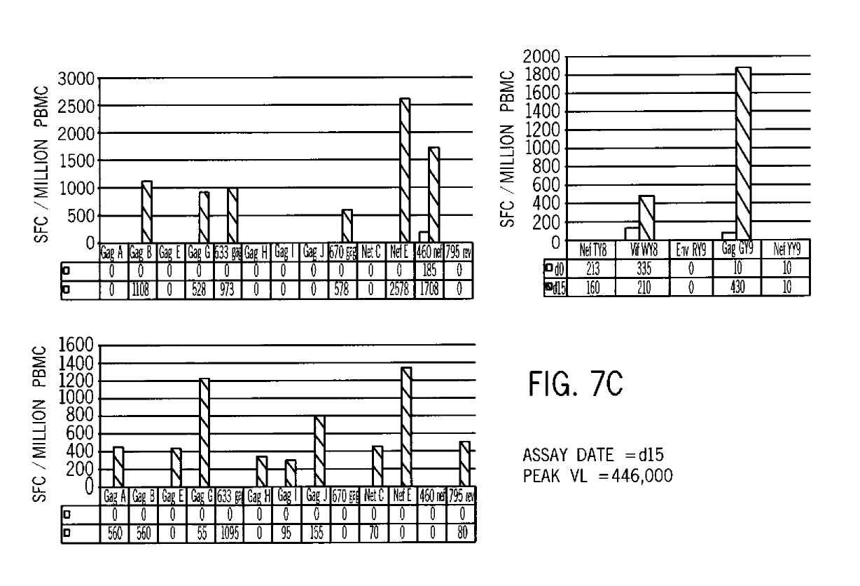

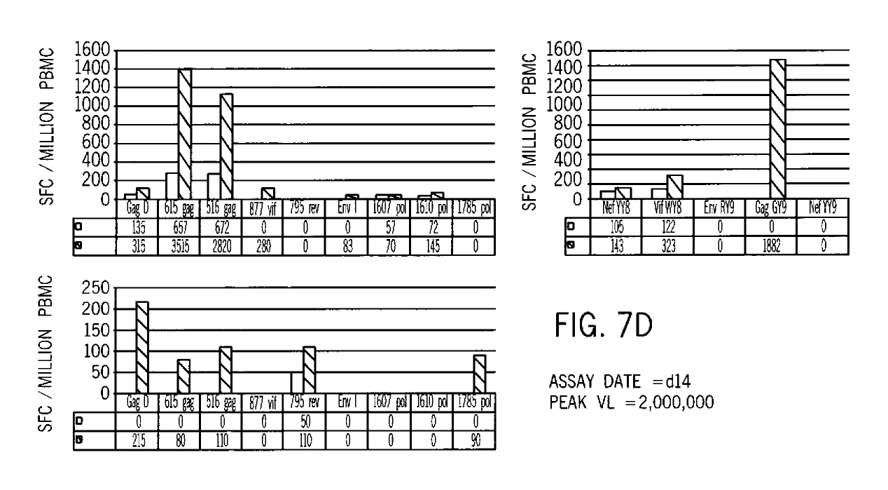

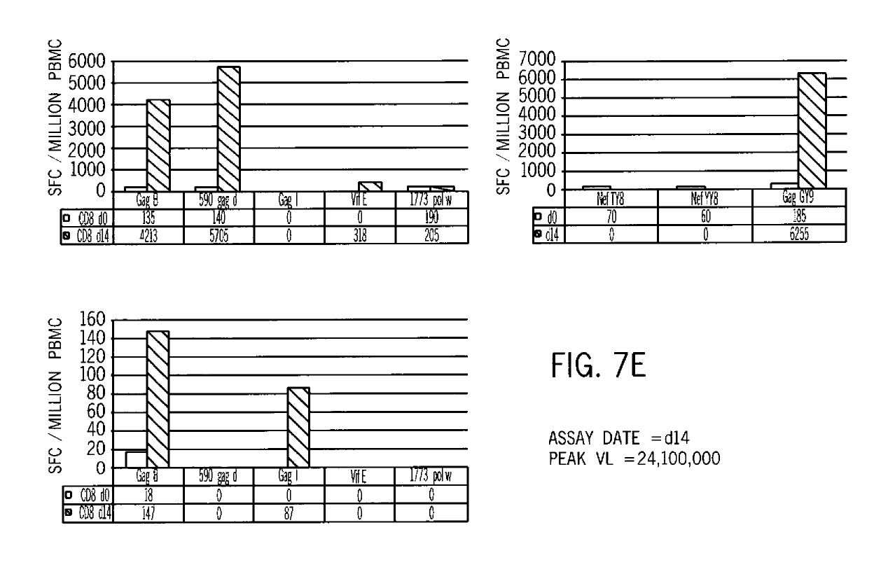

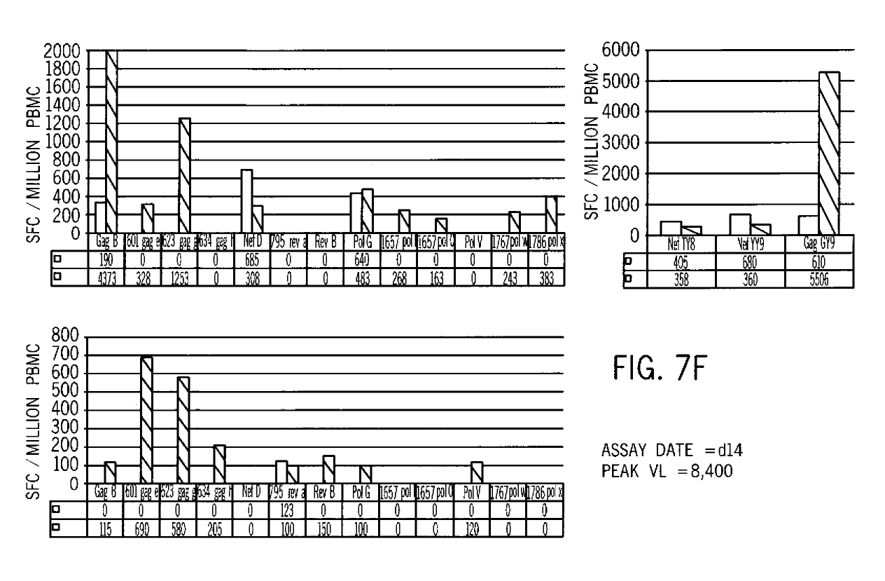

Anamnestic immune responses. All infected vaccinees experienced post-challenge expansions of vaccine-induced immune responses (FIG. 3, FIG. 7). Post challenge, anamnestic vaccine-induced responses was measured since a set of peptides that matched the sequences of SIVsmE660 was not available. Therefore, the only T cell responses examined were those against regions of the virus that had previously been observed during the vaccination phase. As a result, there were fewer responses in this challenge phase to assess and both whole PBMC and CD8-depleted PBMC IFNγ Elispots were performed using all of the reactive peptides seen 14-21 days after Ad5 vaccination. Both CD4 and CD8 responses were assessed very early after challenge, before CD4 responses diminished and prior to the onset of de novo challenge virus-specific responses. Seven of eight vaccinees showed evidence of anamnestic CD4+ and CD8+ T cell responses expanded by the heterologous challenge virus (FIG. 3, FIG. 7B-F). One vaccinee (r00061) mounted only a modest CD8+ T cell response against the challenge virus, commensurate with its low viral load (FIG. 2A, FIG. 7A). Vaccine-induced cellular immune responses were, therefore, recalled effectively, with over 50% of the vaccine-induced responses expanded in the acute phase of infection (Table 6).

High frequency CD4 responses were observed in five of the six vaccinees that successfully controlled replication of the heterologous challenge virus (Table 6). Gag was the main target of both these CD4 responses, and the anamnestic CD8 responses, perhaps due to its conservation between SIVmac239 and SIVsmE660. However, that explanation should also hold true for Pol, but anamnestic cellular responses against this conserved region were a fraction of those against Gag. Nef and Vif also served as CD8+ targets, but largely failed to induce anamnestic CD4 responses.

### Discussion

The level of containment of viral replication observed in the present study suggests that vaccine-induced T-cell responses might indeed be more effective against the AIDS virus than previously considered. SIV vaccines based solely on inducing cellular immune responses (i.e., no Env in the vaccine) have been shown to reduce both acute and chronic phase viral replication of homologous challenge viruses [34, 56]. Unfortunately, however, viral replication exceeded one million copies/ml during the acute phase in these earlier studies using homologous viral challenges, suggesting that it might be difficult to control acute phase viral replication with vaccine-induced T cell responses alone. Encouragingly, these new results indicate that vaccine-induced T cell responses alone can control replication of a heterologous virus during both the acute and the chronic phases of infection even after a heterologous challenge.

Given the low viral loads in vaccinee r00061, it is possible that this animal was not productively infected (FIG. 2A). Soon after the first challenge, viral RNA was detected in plasma from this animal at days 9, 11 and 15. Virus also was re-amplified from frozen RNA extracted on these three days. Additionally, a transient increase in 8 of 13 vaccine-induced CD8+ T cell responses (FIG. 7A) was observed. However, two other independent laboratories were unable to amplify virus from shipped frozen samples from this animal. These two laboratories did amplify virus from a single time point (but not all time points) in shipped frozen plasma samples from vaccinee r02103, another animal with low acute phase viral loads. This vaccinee was clearly infected given the expansion of 12 of 22 CD8+ and CD4+ vaccine-induced anamnestic T cell responses at day 21 post-infection (FIG. 3A). The Mamu-A*02/Gag GY9-specific T cell response expanded from 102 SFC/10⁶ PBMC one month prior to challenge to 2,525 SFC/10⁶ PBMC 21 days after infection in this vaccinee (FIG. 3A). Virus could not be isolated from activated, CD8-depleted, PBMC from r00061 or r02103 on three different occasions. By contrast, virus from PBMC of two of the other vaccinees, (r02114 and r01099, with higher acute phase viral loads), was routinely cultured. Thus, only seven of eight vaccines may have been productively infected. By contrast, all naïve control animals were productively infected.

Much of the attractiveness of vaccines that would elicit broadly-reactive neutralizing antibodies relates to the ability of such antibodies to prevent infection or to limit viral replication during acute infection. This would diminish acute pathogenesis, reduce the generation of genetic diversity of the virus, and as an associated benefit, reduce the prospect of secondary transmission during acute infection, a time when higher levels of viremia are associated with increased transmission [21, 22, 44]. The present results suggest that, vaccine-induced cellular immunity, in the absence of neutralizing antibody, may also be able to achieve these same important objectives. More than half of the vaccinated animals had peak viral loads of less than 80,000 vRNA copy Eq/ml. Achievement of this level of control of a heterologous challenge virus during the acute phase represents an encouraging new benchmark in the evaluation of prophylactic AIDS vaccines.

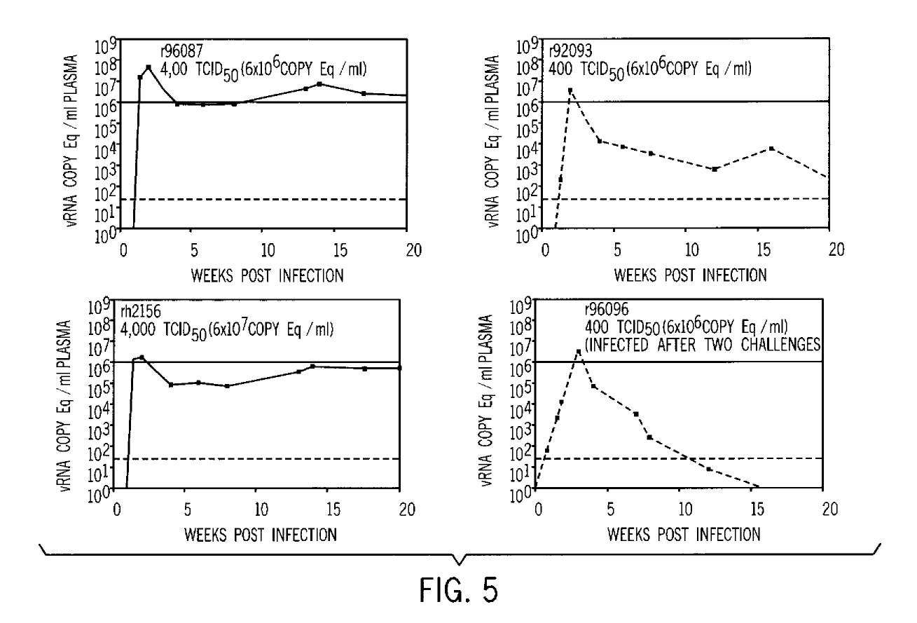

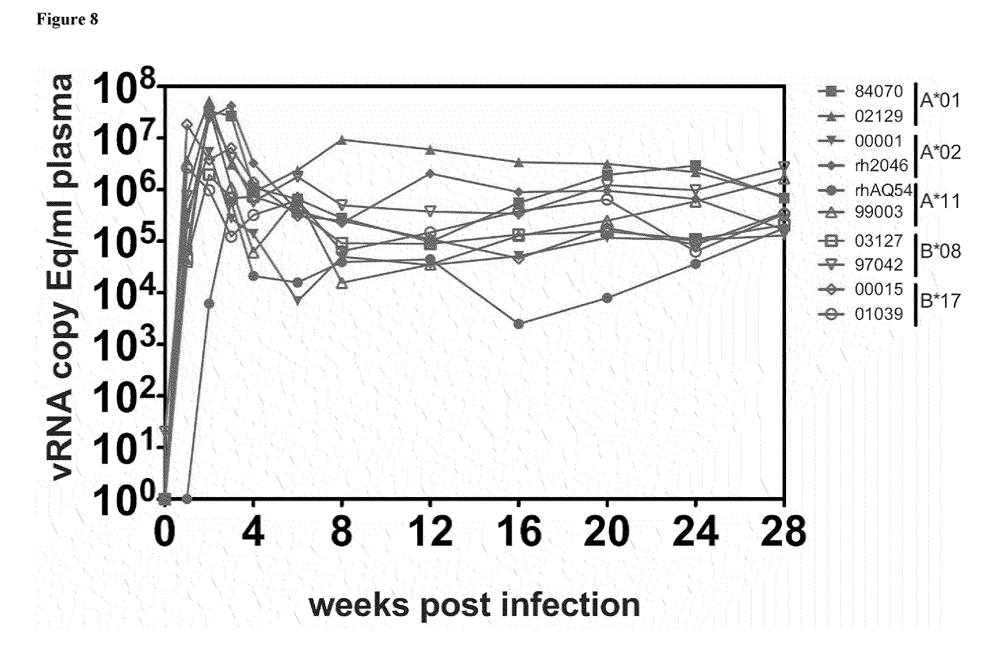

The mucosal challenge model employed in the present study recapitulates the results of human mucosal exposures leading to clinical HIV-1 infections, and the vaccine regimen used provided dramatic protective effects against this challenge that were greater than those seen in other studies. However, as there is less experience with this repeated mucosal heterologous challenge model than some other challenge systems, it needs to be considered whether the protection observed in the present study was a function of superior vaccine efficacy or potentially a result of a less rigorous challenge than used in other studies. Unlike clonal SIV challenge stocks (e.g. SIVmac239), challenge stocks of virus swarms like SIVsmE660 can vary in their pathogenicity depending on how they are propagated. Indeed in recently published studies, five of eight naïve animals and four of six naïve animals controlled SIVsmE660 replication to undetectable levels [32, 61]. However, these do not represent typical results for SIVsmE660 challenge since only 3 of 29 Indian rhesus macaques controlled replication of SIVsmE660 to undetectable levels in many previous studies [2, 15, 16, 23, 24, 27, 43, 50, 57]. The same stock of SIVsmE660 used in the present study has been used previously to intravenously challenge 10 naïve Indian rhesus macaques expressing a variety of MHC class I alleles, including six animals that expressed the “protective” alleles, Mamu-A*01, -B*08 and -B*17. (FIG. 8 and reference 45). All control animals became infected after one intravenous challenge with 100 TCID₅₀ of this virus stock and the mean peak plasma viremia during the acute phase was 5.1×10⁶ vRNA copy Eq/ml. All ten of the naïve control animals had >100,000 vRNA copy Eq/ml at 28 weeks post challenge (FIG. 8). Additionally, no “protective” effects were observed for any of the three MHC class I alleles after SIVsmE660 challenge of these naïve control animals. Furthermore, in the current study, only one of 12 of rectally challenged naive macaques has controlled SIVsmE660 replication to undetectable levels (animal r96096, FIG. 5). The utilized stock of SIVsmE660, therefore, appears to be pathogenic and remarkably consistent from animal to animal after both intravenous and rectal challenge.

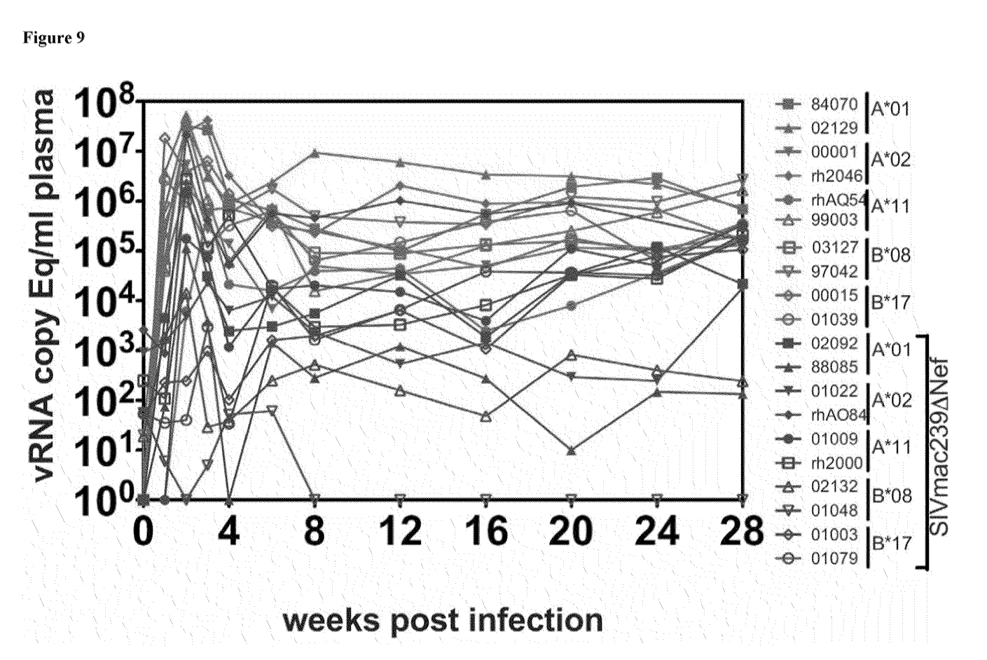

Despite the robust nature of SIVsmE660, it is formally possible that, like SHIV89.6P, SIVsmE660 may represent a less than stringent vaccine challenge virus, yielding misleadingly encouraging results in vaccine studies [6, 7, 17, 33]. Vaccines designed to induce cellular immune responses only (i.e. using Gag/Pol and not Env) have had limited success at reducing acute phase plasma viremia of SIVsmE660 below 1×10⁶ vRNA copy Eq/ml [16, 43, 50]. Furthermore, vaccination using attenuated SIVmac239, the best current vaccine, has shown limited ability to control heterologous SIVsmE660 replication during the acute phase [2, 45, 57]. These previous results are commensurate with recent experiments using SIVmac239ΔNef. Ten Indian rhesus macaques were vaccinated with SIVmac239ΔNef and subsequently challenged intravenously with SIVsmE660 using the stock of virus employed in the current study. Of these 10 SIVmac239ΔNef-vaccinated animals, only four showed some measure of control during the acute phase (FIG. 9), all four of these expressed the protective alleles Mamu-B*08 or Mamu-B*17. Thus the robust pathogenic stock of SIVsmE660 used in the current experiments appears to represent a rigorous challenge for vaccine studies.

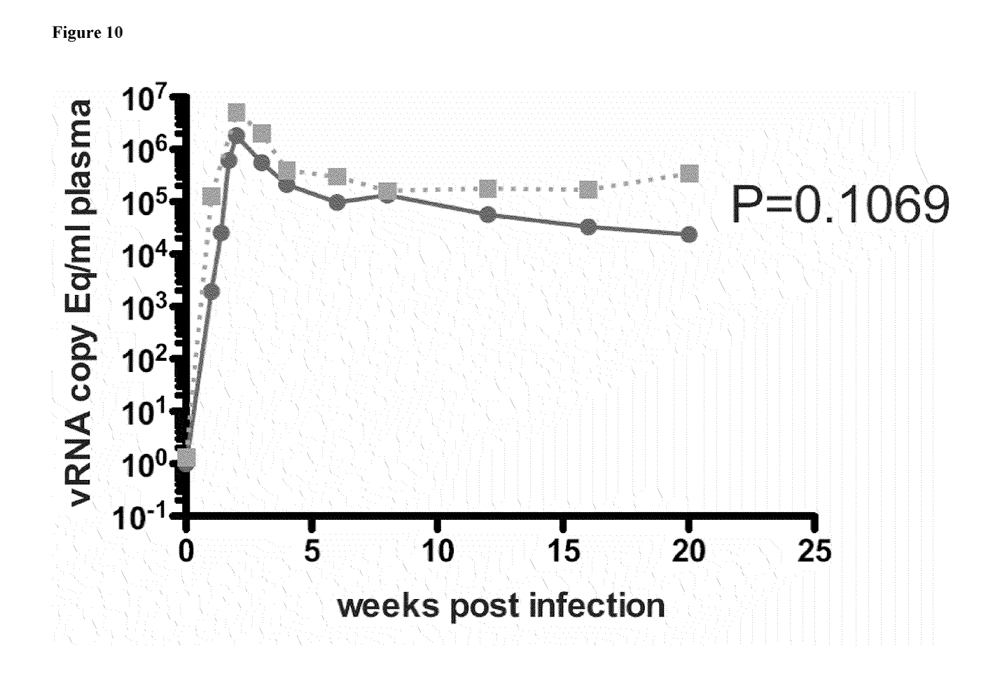

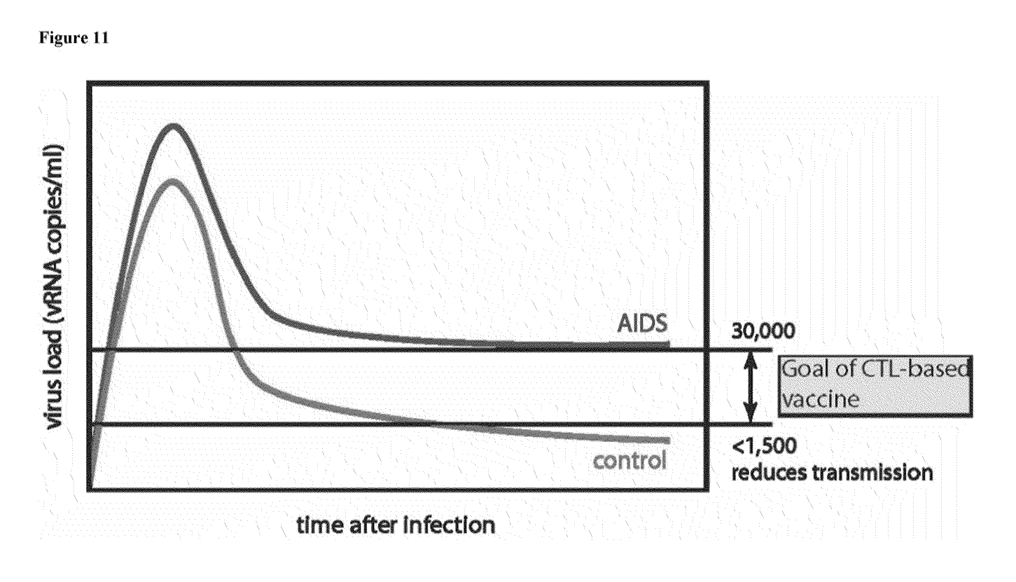

Non-human primate challenge models using pathogenic SIVs for challenge have been criticized for being too stringent. Challenges using the pathogenic viruses SIVmac239 and SIVmac251 often result in plasma viral concentrations of greater than 500,000 vRNA copy Eq/ml in the chronic phase [3-5, 8, 53]. The utilized stock of SIVsmE660 given intravenously resulted in plasma viremia in excess of 100,000 vRNA copy Eq/ml in the chronic phase [45]. Here, a novel mucosal challenge regimen with a heterologous virus swarm was utilized in order to mimic the results of typical mucosal HIV exposure resulting in infection in humans. Macaques were successfully infected with 1-4 variants, replicating the results that have been observed in HIV-infected humans. Mean peak viral loads of 2.5×10⁶ vRNA copy Eq/ml and 80,000 vRNA copy Eq/ml in the chronic phase were observed in the control, naïve, animals. It has been suggested that the intrarectal challenge method utilized might also have resulted in a less stringent challenge. However, when the viral loads in this study are compared with those in Reynolds et al, in which SIVsmE660 from the same stock was given i.v., there was no statistical difference between the viral loads (FIG. 10). These values are similar to average peak and chronic phase viral loads in humans [38, 46]. Therefore, by several key measures HIV exposure in humans was mimicked in the present study.

It will be important to calibrate the ability of other vaccine regimens to control replication of SIVsmE660 after the challenge regimen employed here. A benchmark of particular interest will be to formally assess whether the Merck Ad5 Gag, Pol, and Nef vaccine regimen, a vaccine approach that was not efficacious in the STEP Study, can reduce viral replication after the type of mucosal heterologous SIVsmE660 challenge employed in the current study. Similarly, it will be critical to investigate whether SIVmac239ANef can control SIVsmE660 replication after low dose mucosal challenge in Indian rhesus macaques that do not express MHC class I alleles associated with control of SIVmac239. Only moderate control of viral replication was observed in SIVmac239ΔNef-vaccinated monkeys after high dose intravenous challenge with this same stock of SIVsmE660 [45]. Interestingly animals expressing the MHC class I alleles Mamu-B*08 and -B*17 showed the best control of this heterologous challenge during acute infection. It is important to note that the majority of SIVmac239ΔNef-vaccinated monkeys has shown complete control of standard highly pathogenic homologous challenges with SIVmac239 or SIVmac251, administered either intravenously or mucosally [14, 48, 58].

The present study appears to be the first application of single genome amplification (SGA)-direct amplicon sequencing [29, 47] to the design and interpretation of an Sly vaccine trial. A titration analysis of 4 naïve animals in this study was combined with 9 additional naïve animals in another study (B. F. K., J Exp Med, in press), to estimate a SIVsmE660 inoculum size that would productively infect animals with <5 viruses, recapitulating the results that characterize the majority of human mucosal HIV exposures resulting in clinical infection. The results in Table 5 confirm that in 10 vaccinated or control animals the numbers of transmitted viruses leading to productive infection was indeed between 1 and 4 (Table 5). There were numerically lower numbers of transmitted viruses in vaccinated compared with control animals, but the difference was not statistically significant. An unexpected finding in the present study was a striking enrichment for G-to-A hypermutation observed in some (but not all) animals productively infected by a single virus. This was observed in vaccinated, control and titration animals. While G-to-A hypermutation in HIV-1 infected humans [29] and in SIVsmE660 and SIVmac251 infected Indian rhesus macaques previously has been observed (B. F. K., J Exp Med, submitted), the extent of G-to-A hypermutation observed in some animals in the present study is unprecedented (e.g., animals r92093, r02103, and r02114 in FIG. 3A). With low-dose virus exposure, infection by single viruses with altered Vif function could lead to correspondingly high levels of APOBEC-mediated hypermutation, thereby affecting virus replication efficiency in naïve and vaccinated animals. Such a result might not be apparent in animals productively infected by multiple viruses if one or more of these exhibited wild-type Vif function and better overall replication fitness.

The breadth and frequency of the vaccine-induced T-cell responses achieved in the present study may have been critical for the enhanced control of replication of this heterologous, pathogenic challenge virus. No other vaccine regimens to date have achieved these frequencies or induced the breadth of T cell responses observed in the current experiments [13, 31, 34, 41, 56]. Whether the cellular immunity elicited by the replication-defective adenovirus HIV-1 vaccine in the STEP trial had comparable breadth and appropriate specificity such that one might have expected protection against the infecting strains in the reported cases, is currently being investigated using fine T cell epitope mapping and viral sequencing. Those results will contribute to a better understanding of the underlying reasons for the lack of vaccine efficacy in the STEP trial and help inform the next steps in HIV-1 vaccine research development.

### References for Example

1. [Anonymous]. 2007. HIV vaccine failure prompts Merck to halt trial. Nature 449:390.

2. Abdel-Motal, U. M., J. Gillis, K. Manson, M. Wyand, D. Montefiori, K. Stefano-Cole, R. C. Montelaro, J. D. Altman, and R. P. Johnson. 2005. Kinetics of expansion of SIV Gag-specific CD8+ T lymphocytes following challenge of vaccinated macaques. Virology 333:226-238.

3. Allen, T. M., P. Jing, B. Calore, H. Horton, D. H. O'Connor, T. Hanke, M. Piekarczyk, R. Ruddersdorf, B. R. Mothe, C. Emerson, N. Wilson, J. D. Lifson, I. M. Belyakov, J. A. Berzofsky, C. Wang, D. B. Allison, D. C. Montefiori, R. C. Desrosiers, S. Wolinsky, K. J. Kunstman, J. D. Altman, A. Sette, A. J. McMichael, and D. I. Watkins. 2002. Effects of cytotoxic T lymphocytes (CTL) directed against a single simian immunodeficiency virus (SIV) Gag CTL epitope on the course of SIVmac239 infection. J Virol 76:10507-10511.

4. Allen, T. M., L. Mortara, B. R. Mothe, M. Liebl, P. Jing, B. Calore, M. Piekarczyk, R. Ruddersdorf, D. H. O'Connor, X. Wang, C. Wang, D. B. Allison, J. D. Altman, A. Sette, R. C. Desrosiers, G. Sutter, and D. I. Watkins. 2002. Tat-vaccinated macaques do not control simian immunodeficiency virus SIVmac239 replication. J Virol 76:4108-4112.

5. Allen, T. M., T. U. Vogel, D. H. Fuller, B. R. Mothe, S. Steffen, J. E. Boyson, T. Shipley, J. Fuller, T. Hanke, A. Sette, J. D. Altman, B. Moss, A. J. McMichael, and D. I. Watkins. 2000. Induction of AIDS virus-specific CTL activity in fresh, unstimulated peripheral blood lymphocytes from rhesus macaques vaccinated with a DNA prime/modified vaccinia virus Ankara boost regimen. J Immunol 164:4968-4978.

6. Amara, R. R., F. Villinger, J. D. Altman, S. L. Lydy, S. P. O'Neil, S. I. Staprans, D. C. Montefiori, Y. Xu, J. G. Herndon, L. S. Wyatt, M. A. Candido, N. L. Kozyr, P. L. Earl, J. M. Smith, H. L. Ma, B. D. Grimm, M. L. Hulsey, J. Miller, H. M. McClure, J. M. McNicholl, B. Moss, and H. L. Robinson. 2001. Control of a mucosal challenge and prevention of AIDS by a multiprotein DNA/MVA vaccine. Science 292:69-74.

7. Barouch, D. H., S. Santra, J. E. Schmitz, M. J. Kuroda, T. M. Fu, W. Wagner, M. Bilska, A. Craiu, X. X. Zheng, G. R. Krivulka, K. Beaudry, M. A. Lifton, C. E. Nickerson, W. L. Trigona, K. Punt, D. C. Freed, L. Guan, S. Dubey, D. Casimiro, A. Simon, M. E. Davies, M. Chastain, T. B. Strom, R. S. Gelman, D. C. Montefiori, M. G. Lewis, E. A. Emini, J. W. Shiver, and N. L. Letvin. 2000. Control of viremia and prevention of clinical AIDS in rhesus monkeys by cytokine-augmented DNA vaccination. Science 290:486-492.

8. Benson, J., C. Chougnet, M. Robert-Guroff, D. Montefiori, P. Markham, G. Shearer, R. C. Gallo, M. Cranage, E. Paoletti, K. Limbach, D. Venzon, J. Tartaglia, and G. Franchini. 1998. Recombinant vaccine-induced protection against the highly pathogenic simian immunodeficiency virus SIV(mac251): dependence on route of challenge exposure. J Virol 72:4170-4182.

9. Borrow, P., H. Lewicki, B. H. Hahn, G. M. Shaw, and M. B. Oldstone. 1994. Virus-specific CD8+ cytotoxic T-lymphocyte activity associated with control of viremia in primary human immunodeficiency virus type 1 infection. J Virol 68:6103-6110.

10. Brown, C. R., M. Czapiga, J. Kabat, Q. Dang, I. Ourmanov, Y. Nishimura, M. A. Martin, and V. M. Hirsch. 2007. Unique pathology in simian immunodeficiency virus-infected rapid progressor macaques is consistent with a pathogenesis distinct from that of classical AIDS. J Virol 81:5594-5606.

11. Buchbinder, S. P., D. V. Mehrotra, A. Duerr, D. W. Fitzgerald, R. Mogg, D. Li, P. B. Gilbert, J. R. Lama, M. Marmor, C. Del Rio, M. J. McElrath, D. R. Casimiro, K. M. Gottesdiener, J. A. Chodakewitz, L. Corey, and M. N. Robertson. 2008. Efficacy assessment of a cell-mediated immunity HIV-1 vaccine (the Step Study): a double-blind, randomised, placebo-controlled, test-of-concept trial. Lancet 372:1881-1893.

12. Burton, D. R., R. C. Desrosiers, R. W. Doms, W. C. Koff, P. D. Kwong, J. P. Moore, G. J. Nabel, J. Sodroski, I. A. Wilson, and R. T. Wyatt. 2004. HIV vaccine design and the neutralizing antibody problem. Nat Immunol 5:233-236.

13. Casimiro, D. R., F. Wang, W. A. Schleif, X. Liang, Z. Q. Zhang, T. W. Tobery, M. E. Davies, A. B. McDermott, D. H. O'Connor, A. Fridman, A. Bagchi, L. G. Tussey, A. J. Bett, A. C. Finnefrock, T. M. Fu, A. Tang, K. A. Wilson, M. Chen, H. C. Perry, G. J. Heidecker, D. C. Freed, A. Carella, K. S. Punt, K. J. Sykes, L. Huang, V. I. Ausensi, M. Bachinsky, U. Sadasivan-Nair, D. I. Watkins, E. A. Emini, and J. W. Shiver. 2005. Attenuation of simian immunodeficiency virus SIVmac239 infection by prophylactic immunization with dna and recombinant adenoviral vaccine vectors expressing Gag. J Virol 79:15547-15555.

14. Daniel, M. D., F. Kirchhoff, S. C. Czajak, P. K. Sehgal, and R. C. Desrosiers. 1992. Protective effects of a live attenuated SIV vaccine with a deletion in the nef gene. Science 258:1938-1941.

15. Davis, N. L., I. J. Caley, K. W. Brown, M. R. Betts, D. M. Irlbeck, K. M. McGrath, M. J. Connell, D. C. Montefioii, J. A. Frelinger, R. Swanstrom, P. R. Johnson, and R. E. Johnston. 2000. Vaccination of macaques against pathogenic simian immunodeficiency virus with Venezuelan equine encephalitis virus replicon particles. J Virol 74:371-378.

16. Egan, M. A., W. A. Charini, M. J. Kuroda, J. E. Schmitz, P. Racz, K. Tenner-Racz, K. Manson, M. Wyand, M. A. Lifton, C. E. Nickerson, T. Fu, J. W. Shiver, and N. L. Letvin. 2000. Simian immunodeficiency virus (SIV) gag DNA-vaccinated rhesus monkeys develop secondary cytotoxic T-lymphocyte responses and control viral replication after pathogenic SIV infection. J Virol 74:7485-7495.

17. Feinberg, M. B., and J. P. Moore. 2002. AIDS vaccine models: challenging challenge viruses. Nat Med 8:207-210.

18. Friedrich, T. C., L. E. Valentine, L. J. Yant, E. G. Rakasz, S. M. Piaskowski, J. R. Furlott, K. L. Weisgrau, B. Burwitz, G. E. May, E. J. Leon, T. Soma, G. Napoe, S. V. r. Capuano, N. A. Wilson, and D. I. Watkins. 2007. Subdominant CD8+ T-cell responses are involved in durable control of AIDS virus replication. J Virol 81:3465-3476.

19. Gaschen, B., J. Taylor, K. Yusim, B. Foley, F. Gao, D. Lang, V. Novitsky, B. Haynes, B. H. Hahn, T. Bhattacharya, and B. Korber. 2002. Diversity considerations in HIV-1 vaccine selection. Science 296:2354-2360.

20. Goldstein, S., W. R. Elkins, W. T. London, A. Hahn, R. Goeken, J. E. Martin, and V. M. Hirsch. 1994. Immunization with whole inactivated vaccine protects from infection by SIV grown in human but not macaque cells. J Med Primatol 23:75-82.

21. Gray, R. H., X. Li, M. J. Wawer, S. J. Gange, D. Serwadda, N. K. Sewankambo, R. Moore, F. Wabwire-Mangen, T. Lutalo, and T. C. Quinn. 2003. Stochastic simulation of the impact of antiretroviral therapy and HIV vaccines on HIV transmission; Rakai, Uganda. AIDS 17:1941-1951.

22. Gray, R. H., M. J. Wawcr, R. Brookmeyer, N. K. Scwankambo, D. Serwadda, F. Wabwire-Mangen, T. Lutalo, X. Li, T. vanCott, and T. C. Quinn. 2001. Probability of HIV-1 transmission per coital act in monogamous, heterosexual, HIV-1-discordant couples in Rakai, Uganda. Lancet 357:1149-1153.

23. Haigwood, N. L., A. Watson, W. F. Sutton, J. McClure, A. Lewis, J. Ranchalis, B. Travis, G. Voss, N. L. Letvin, S. L. Hu, V. M. Hirsch, and P. R. Johnson. 1996. Passive immune globulin therapy in the SIV/macaque model: early intervention can alter disease profile. Immunol Lett 51:107-114.

24. Hirsch, V. M., T. R. Fuerst, G. Sutter, M. W. Carroll, L. C. Yang, S. Goldstein, M. J. Piatak, W. R. Elkins, W. G. Alvord, D. C. Montefiori, B. Moss, and J. D. Lifson. 1996. Patterns of viral replication correlate with outcome in simian immunodeficiency virus (SIV)-infected macaques: effect of prior immunization with a trivalent SIV vaccine in modified vaccinia virus Ankara. J Virol 70:3741-3752.

25. Hirsch, V. M., and P. R. Johnson. 1994. Pathogenic diversity of simian immunodeficiency viruses. Virus Res 32:183-203.

26. Jin, X., D. E. Bauer, S. E. Tuttleton, S. Lewin, A. Gettie, J. Blanchard, C. E. Irwin, J. T. Safrit, J. Mittler, L. Weinberger, L. G. Kostrikis, L. Zhang, A. S. Perelson, and D. D. Ho. 1999. Dramatic rise in plasma viremia after CD8(+) T cell depletion in simian immunodeficiency virus-infected macaques. J Exp Med 189:991-998.

27. Johnston, R. E., P. R. Johnson, M. J. Connell, D. C. Montefiori, A. West, M. L. Collier, C. Cecil, R. Swanstrom, J. A. Frelinger, and N. L. Davis. 2005. Vaccination of macaques with SIV immunogens delivered by Venezuelan equine encephalitis virus replicon particle vectors followed by a mucosal challenge with SIVsmE660. Vaccine 23:4969-4979.

28. Kaizu, M., G. J. Borchardt, C. E. Glidden, D. L. Fisk, J. T. Loffredo, D. I. Watkins, and W. M. Rehrauer. 2007. Molecular typing of major histocompatibility complex class I alleles in the Indian rhesus macaque which restrict SIV CD8+ T cell epitopes. Immunogenetics 59:693-703.

29. Keele, B. F., E. E. Giorgi, J. F. Salazar-Gonzalez, J. M. Decker, K. T. Pham, M. G. Salazar, C. Sun, T. Grayson, S. Wang, H. Li, X. Wei, C. Jiang, J. L. Kirchherr, F. Gao, J. A. Anderson, L. H. Ping, R. Swanstrom, G. D. Tomaras, W. A. Blattner, P. A. Goepfert, J. M. Kilby, M. S. Saag, E. L. Delwart, M. P. Busch, M. S. Cohen, D. C. Montefiori, B. F. Haynes, B. Gaschen, G. S. Athreya, H. Y. Lee, N. Wood, C. Seoighe, A. S. Perelson, T. Bhattacharya, B. T. Korber, B. H. Hahn, and G. M. Shaw. 2008. Identification and characterization of transmitted and early founder virus envelopes in primary HIV-1 infection. Proc Natl Acad Sci USA 105:7552-7557.

30. Koup, R. A., J. T. Safrit, Y. Cao, C. A. Andrews, G. McLeod, W. Borkowsky, C. Farthing, and D. D. Ho. 1994. Temporal association of cellular immune responses with the initial control of viremia in primary human immunodeficiency virus type 1 syndrome. J Virol 68:4650-4655.

31. Letvin, N. L., J. R. Mascola, Y. Sun, D. A. Gorgone, A. P. Buzby, L. Xu, Z. Y. Yang, B. Chakrabarti, S. S. Rao, J. E. Schmitz, D. C. Montefiori, B. R. Barker, F. L. Bookstein, and G. J. Nabel. 2006. Preserved CD4+ central memory T cells and survival in vaccinated SIV-challenged monkeys. Science 312:1530-1533.

32. Letvin, N. L., S. S. Rao, V. Dang, A. P. Buzby, B. Korioth-Schmitz, D. Dombagoda, J. G. Parvani, R. H. Clarke, L. Bar, K. R. Carlson, P. A. Kozlowski, V. M. Hirsch, J. R. Mascola, and G. J. Nabel. 2007. No evidence for consistent virus-specific immunity in simian immunodeficiency virus-exposed, uninfected rhesus monkeys. J Virol 81:12368-12374.

33. Lifson, J. D., and M. A. Martin. 2002. One step forwards, one step back. Nature 415:272-273.

34. Liu, J., K. L. O'Brien, D. M. Lynch, N. L. Simmons, A. La Porte, A. M. Riggs; P. Abbink, R. T. Coffey, L. E. Grandpre, M. S. Seaman, G. Landucci, D. N. Forthal, D. C. Montefiori, A. Carville, K. G. Mansfield, M. J. Havenga, M. G. Pau, J. Goudsmit, and D. H. Barouch. 2009. Immune control of an SIV challenge by a T-cell-based vaccine in rhesus monkeys. Nature 457:87-91.

35. Loffredo, J. T., B. J. Burwitz, E. G. Rakasz, S. P. Spencer, J. J. Stephany, J. P. Vela, S. R. Martin, J. Reed, S. M. Piaskowski, J. Furlott, K. L. Weisgrau, D. S. Rodrigues, T. Soma, G. Napoe, T. C. Friedrich, N. A. Wilson, E. G. Kailas, and D. I. Watkins. 2007. The antiviral efficacy of simian immunodeficiency virus-specific CD8+ T cells is unrelated to epitope specificity and is abrogated by viral escape. J Virol 81:2624-2634.

36. Loffredo, J. T., J. Maxwell, Y. Qi, C. E. Glidden, G. J. Borchardt, T. Soma, A. T. Bean, D. R. Beal, N. A. Wilson, W. M. Rehrauer, J. D. Lifson, M. Carrington, and D. I. Watkins. 2007. Mamu-B*08-positive macaques control simian immunodeficiency virus replication. J Virol 81:8827-8832.

37. Loffredo, J. T., J. Sidney, C. Wojewoda, E. Dodds, M. R. Reynolds, G. Napoe, B. R. Mothe, D. H. O'Connor, N. A. Wilson, D. I. Watkins, and A. Sette. 2004. Identification of seventeen new simian immunodeficiency virus-derived CD8+ T cell epitopes restricted by the high frequency molecule, Mamu-A*02, and potential escape from CTL recognition. J Immunol 173:5064-5076.

38. Lyles, R. H., A. Munoz, T. E. Yamashita, H. Bazmi, R. Detels, C. R. Rinaldo, J. B. Margolick, J. P. Phair, and J. W. Mellors. 2000. Natural history of human immunodeficiency virus type 1 viremia after seroconversion and proximal to AIDS in a large cohort of homosexual men. Multicenter AIDS Cohort Study. J Infect Dis 181:872-880.

39. Matano, T., R. Shibata, C. Siemon, M. Connors, H. C. Lane, and M. A. Martin. 1998. Administration of an anti-CD8 monoclonal antibody interferes with the clearance of chimeric simian/human immunodeficiency virus during primary infections of rhesus macaques. J Virol 72:164-169.

40. McDermott, A. B., J. Mitchen, S. Piaskowski, I. De Souza, L. J. Yant, J. Stephany, J. Furlott, and D. I. Watkins. 2004. Repeated low-dose mucosal simian immunodeficiency virus SIVmac239 challenge results in the same viral and immunological kinetics as high-dose challenge: a model for the evaluation of vaccine efficacy in nonhuman primates. J Virol 78:3140-3144.

41. McDermott, A. B., D. H. O'Connor, S. Fuenger, S. Piaskowski, S. Martin, J. Loffredo, M. Reynolds, J. Reed, J. Furlott, T. Jacoby, C. Riek, E. Dodds, K. Krebs, M. E. Davies, W. A. Schleif, D. R. Casimiro, J. W. Shiver, and D. I. Watkins. 2005. Cytotoxic T-lymphocyte escape does not always explain the transient control of simian immunodeficiency virus SIVmac239 viremia in adenovirus-boosted and DNA-primed Mamu-A*01-positive rhesus macaques. J Virol 79:15556-15566.

42. McElrath, M. J., S. C. De Rosa, Z. Moodie, S. Dubey, L. Kierstead, H. Janes, O. D. Defawe, D. K. Carter, J. Hural, R. Akondy, S. P. Buchbinder, M. N. Robertson, D. V. Mehrotra, S. G. Self, L. Corey, J. W. Shiver, and D. R. Casimiro. 2008. HIV-1 vaccine-induced immunity in the test-of-concept Step Study: a case-cohort analysis. Lancet 372:1894-1905.

43. Ourmanov, I., C. R. Brown, B. Moss, M. Carroll, L. Wyatt, L. Pletneva, S. Goldstein, D. Venzon, and V. M. Hirsch. 2000. Comparative efficacy of recombinant modified vaccinia virus Ankara expressing simian immunodeficiency virus (SIV) Gag-Pol and/or Env in macaques challenged with pathogenic SIV. J Virol 74:2740-2751.

44. Quinn, T. C., M. J. Wawer, N. Sewankambo, D. Serwadda, C. Li, F. Wabwire-Mangen, M. O. Meehan, T. Lutalo, and R. H. Gray. 2000. Viral load and heterosexual transmission of human immunodeficiency virus type 1. Rakai Project Study Group. N Engl J Med 342:921-929.

45. Reynolds, M. R., A. M. Weiler, K. L. Weisgrau, S. M. Piaskowski, J. R. Furlott, J. T. Weinfurter, M. Kaizu, T. Soma, E. J. Leon, C. MacNair, D. P. Leaman, M. B. Zwick, E. Gostick, S. K. Musani, D. A. Price, T. C. Friedrich, E. G. Rakasz, N. A. Wilson, A. B. McDermott, R. Boyle, D. B. Allison, D. R. Burton, W. C. Koff, and D. I. Watkins. 2008. Macaques vaccinated with live-attenuated SIV control replication of heterologous virus. J Exp Med 205:2537-2550.

46. Rosenberg, E. S., M. Altfeld, S. H. Poon, M. N. Phillips, B. M. Wilkes, R. L. Eldridge, G. K. Robbins, R. T. D'Aquila, P. J. Goulder, and B. D. Walker. 2000. Immune control of HIV-1 after early treatment of acute infection. Nature 407:523-526.

47. Salazar-Gonzalez, J. F., E. Bailes, K. T. Pham, M. G. Salazar, M. B. Guffey, B. F. Keele, C. A. Derdeyn, P. Farmer, E. Hunter, S. Allen, O. Manigart, J. Mulenga, J. A. Anderson, R. Swanstrom, B. F. Haynes, G. S. Athreya, B. T. Korber, P. M. Sharp, G. M. Shaw, and B. H. Hahn. 2008. Deciphering human immunodeficiency virus type 1 transmission and early envelope diversification by single-genome amplification and sequencing. J Virol 82:3952-3970.

48. Schmitz, J. E., R. P. Johnson, H. M. McClure, K. H. Manson, M. S. Wyand, M. J. Kuroda, M. A. Lifton, R. S. Khunkhun, K. J. McEvers, J. Gillis, M. Piatak, J. D. Lifson, G. Grosschupff, P. Racz, K. Tenner-Racz, E. P. Rieber, K. Kuus-Reichel, R. S. Gelman, N. L. Letvin, D. C. Montefiori, R. M. Ruprecht, R. C. Desrosiers, and K. A. Reimann. 2005. Effect of CD8+ lymphocyte depletion on virus containment after simian immunodeficiency virus SIVmac251 challenge of live attenuated SIVmac239delta3-vaccinated rhesus macaques. J Virol 79:8131-8141.

49. Sekaly, R. P. 2008. The failed HIV Merck vaccine study: a step back or a launching point for future vaccine development? J Exp Med 205:7-12.

50. Seth, A., I. Ourmanov, J. E. Schmitz, M. J. Kuroda, M. A. Lifton, C. E. Nickerson, L. Wyatt, M. Carroll, B. Moss, D. Venzon, N. L. Letvin, and V. M. Hirsch. 2000. Immunization with a modified vaccinia virus expressing simian immunodeficiency virus (SIV) Gag-Pol primes for an anamnestic Gag-specific cytotoxic T-lymphocyte response and is associated with reduction of viremia after SIV challenge. J Virol 74:2502-2509.

51. Shiver, J. W., T. M. Fu, L. Chen, D. R. Casimiro, M. E. Davies, R. K. Evans, Z. Q. Zhang, A. J. Simon, W. L. Trigona, S. A. Dubey, L. Huang, V. A. Harris, R. S. Long, X. Liang, L. Handt, W. A. Schleif, L. Zhu, D. C. Freed, N. V. Persaud, L. Guan, K. S. Punt, A. Tang, M. Chen, K. A. Wilson, K. B. Collins, G. J. Heidecker, V. R. Fernandez, H. C. Perry, J. G. Joyce, K. M. Grimm, J. C. Cook, P. M. Keller, D. S. Kresock, H. Mach, R. D. Troutman, L. A. Isopi, D. M. Williams, Z. Xu, K. E. Bohannon, D. B. Volkin, D. C. Montefiori, A. Miura, G. R. Krivulka, M. A. Lifton, M. J. Kuroda, J. E. Schmitz, N. L. Letvin, M. J. Caulfield, A. J. Bet, R. Youil, D. C. Kaslow, and E. A. Emini. 2002. Replication-incompetent adenoviral vaccine vector elicits, effective anti-immunodeficiency-virus immunity. Nature 415:331-335.

52. Thompson, J. D., D. G. Higgins, and T. J. Gibson. 1994. CLUSTAL W: improving the sensitivity of progressive multiple sequence alignment through sequence weighting, position-specific gap penalties and weight matrix choice. Nucleic Acids Res 22:4673-4680.

53. Vogel, T. U., M. R. Reynolds, D. H. Fuller, K. Vielhuber, T. Shipley, J. T. Fuller, K. J. Kunstman, G. Sutter, M. L. Marthas, V. Erfle, S. M. Wolinsky, C. Wang, D. B. Allison, E. W. Rud, N. Wilson, D. Montefiori, J. D. Altman, and D. I. Watkins. 2003. Multispecific vaccine-induced mucosal cytotoxic T lymphocytes reduce acute-phase viral replication but fail in long-term control of simian immunodeficiency virus SIVmac239. J Virol 77:13348-13360.

54. Walker, B. D., and D. R. Burton. 2008. Toward an AIDS vaccine. Science 320:760-764.

55. Watkins, D. I., D. R. Burton, E. G. Kailas, I. P. Moore, and W. C. Koff. 2008. Nonhuman primate models and the failure of the Merck HIV-1 vaccine in humans. Nat Med 14:617-621.

56. Wilson, N. A., J. Reed, G. S. Napoe, S. Piaskowski, A. Szymanski, J. Furlott, E. J. Gonzalez, L. J. Yant, N. J. Maness, G. E. May, T. Soma, M. R. Reynolds, E. Rakasz, R. Rudersdorf, A. B. McDermott, D. H. O'Connor, T. C. Friedrich, D. B. Allison, A. Patki, L. J. Picker, D. R. Burton, J. Lin, L. Huang, D. Patel, G. Heindecker, J. Fan, M. Citron, M. Horton, F. Wang, X. Liang, J. W. Shiver, D. R. Casimiro, and D. I. Watkins. 2006. Vaccine-induced cellular immune responses reduce plasma viral concentrations after repeated low-dose challenge with pathogenic simian immunodeficiency virus SIVmac239. J Virol 80:5875-5885.

57. Wyand, M. S., K. Manson, D. C. Montefiori, J. D. Lifson, R. P. Johnson, and R. C. Desrosiers. 1999. Protection by live, attenuated simian immunodeficiency virus against heterologous challenge. J Virol 73:8356-8363.

58. Wyand, M. S., K. H. Manson, M. Garcia-Moll, D. Montefiori, and R. C. Desrosiers. 1996. Vaccine protection by a triple deletion mutant of simian immunodeficiency virus. J Virol 70:3724-3733.

59. Yang, O. O. 2008. Retracing our STEP towards a successful CTL-based HIV-1 vaccine. Vaccine 26:3138-3141.

60. Yant, L. J., T. C. Friedrich, R. C. Johnson, G. E. May, N. J. Maness, A. M. Enz, J. D. Lifson, D. H. O'Connor, M. Carrington, and D. I. Watkins. 2006. The high-frequency major histocompatibility complex class I allele Mamu-B* 17 is associated with control of simian immunodeficiency virus SIVmac239 replication. J Virol 80:5074-5077.

61. Yeh, W. W., P. Jaru-Ampornpan, D. Nevidomskyte, M. Asmal, S. S. Rao, A. P. Buzby, D. C. Montefiori, B. T. Korber, and N. L. Letvin. 2009. Partial protection of SIV-infected rhesus monkeys against superinfection with a heterologous SIV isolate. J Virol 83:2686-2696.

### Example 2

Reference is made to Martins et al., “T-Cell Correlates of Vaccine Efficacy after a Heterologous Simian Immunodeficiency Virus Challenge,” J. Virol. Doi:10.1128/JVI.02365-09, published online ahead of print on 17 Feb. 2010, the content of which is incorporated by reference herein it its entirety.

### Summary

Determining the “correlates of protection” is one of the challenges in human immunodeficiency virus (HIV) vaccine design. To date, T-cell based AIDS vaccines have been evaluated with validated techniques that measure the number of CD8+ T-cells in the blood that secrete cytokines, mainly IFN-γ, in response to synthetic peptides. Despite providing accurate and reproducible measurements of immunogenicity, these methods do not directly assess antiviral function and thus may not identify protective CD8+ T-cell responses. To better understand the correlates of vaccine efficacy, the immune responses elicited by a successful T-cell based vaccine against a heterologous simian immunodeficiency virus (SIV) challenge was analyzed. Correlates of protection were searched using a viral suppression assay (VSA) and IFN-γ ELISPOT. While the VSA measured in vitro suppression, it did not predict the outcome of the vaccine trial. However, several aspects of the vaccine-induced T-cell response that were associated with improved outcome after challenge were identified. Of note, broad vaccine-induced pre-challenge T-cell responses directed against Gag and Vif correlated with lower viral loads and higher CD4+ lymphocyte counts. These results may be relevant for the development of T-cell based AIDS vaccines as they indicate that broad epitope-specific repertoires elicited by vaccination might serve as a correlate of vaccine efficacy. Furthermore, this study demonstrates that certain viral proteins may be more effective than others as vaccine immunogens.

### Introduction

CD8+ T-cells play a vital role in immune control of human immunodeficiency virus (HIV) infection [2, 12, 20, 22, 27, 41]. As a result, many vaccine candidates under development aim to induce cellular immune responses against the virus [16, 58, 61]. These strategies have been evaluated based on their immunogenicity, that is, their capacity to induce IFN-γ production by WV-specific CD8+ T-cells [15, 60]. However, it is becoming increasingly clear that this measure of T-cell responses cannot solely be used to predict vaccine efficacy [11, 15, 44, 60, 61]. A technique for measuring T-cell immunity that will reflect effective antiviral responses in vivo is desirable.

Two assays have been validated to assess T-cell responses in HIV vaccine trials [15]. Based on IFN-γ secretion by antigen-stimulated T-cells, ELISPOT provides a quantitative measure of circulating virus-specific T-lymphocytes. Similarly, intracellular cytokine staining (ICS) employs flow cytometric methods to identify responding cells in both the CD4+ and CD8+ T-cell compartments. Using these assays, it has been shown that control of viral replication in HIV-infected individuals is affected by which viral proteins are targeted by the immune system [31]. Gag-directed cellular responses, for instance, have been repeatedly associated with lower viral loads [9, 17, 31, 52], whereas T-cell responses targeting Env have actually been linked to higher viremia [31, 49]. These studies are directly applicable to vaccine development, as they provide information on which immunogens might induce the most effective T-cell responses.

Despite being invaluable tools for determining cellular reactivity to HIV antigens, ELISPOT and ICS do not directly measure antiviral function [4, 60, 61]. These assays typically involve loading cells with supra-physiological concentrations of synthetic peptides containing T-cell epitopes. These conditions bypass the antigen trafficking and processing pathways that operate in naturally infected cells to allow for CD8+ T-cell recognition. Importantly, it has been demonstrated that the ability of CD8+ T-cells to recognize epitope variants in ELISPOT does not correlate with their capacity to suppress replication of variant viruses in tissue culture [7, 8, 57]. Furthermore, cytokine secretion does not always identify CD8+ T-cells capable of suppressing viral replication in vitro [14]. Most importantly, the ability to produce IFN-γ in an ELISPOT assay does not correlate with control of viral replication [1].

A few alternative approaches have been proposed as surrogate markers of efficacious T-cell responses. The ability of T-cells to perform multiple functions upon antigen encounter, commonly referred to as polyfunctionality, has been correlated with enhanced immune control of HIV-1 infection [10, 56]. However, it is unknown to what extent polyfunctional T-cells are a cause or effect of viral containment. A more direct analysis of CD8+ T-cell-mediated antiviral activity has come from studies measuring in vitro suppression of HIV-1 replication in autologous CD4+ targets [18, 47, 55]. Using this parameter, two groups have demonstrated that individuals who spontaneously control HIV-1 replication (termed elite controllers) have CD8+ T-cells that suppress viral replication more efficiently than those obtained from patients progressing to AIDS [47, 55]. Thus, assays based on the HIV-suppressive capacity of CD8+ T-cells might be useful for screening potential AIDS T-cell based vaccine strategies.

Recently, eight Indian rhesus macaques were vaccinated with a DNA prime, Ad5 boost regimen encoding all SIVmac239 proteins, except for Envelope [59]. After repeated low-dose mucosal challenge with the heterologous swarm virus SIVsmE660, most vaccinated animals controlled acute phase viremia [59]. Indeed, six of the eight vaccinees have no detectable virus one year after challenge. Here, the correlates of this vaccine-induced protection were analyzed. Surprisingly, the ability of pre-challenge vaccine-elicited CD8+ T-cells to suppress viral replication in vitro did not predict control of viral replication in vivo. However, a comprehensive analysis of cellular immune responses in vaccinees revealed several positive correlations with successful outcome after challenge. In particular, the breadth of T-cell responses directed against Gag and Vif correlated with lower viral loads and preservation of CD4+ T-cell numbers after a heterologous challenge.

### Methods

Animals. The animals in this study were Indian rhesus macaques (Macaca mulatta) from the Wisconsin National Primate Research Center colony. They were typed for major histocompatibility complex class I (MHC-I) alleles Mamu-A*01, Mamu-A*02, Mamu-A*08, Mamu-A* 11, Mamu-B*01, Mamu-B*03, Mamu-B*04, Mamu-B*08, Mamu-B*17, and Mamu-B*29 by sequence-specific PCR analysis [28, 36]. Animals that were Mamu-A*02 positive were chosen for this study, whereas those that were positive for Mamu-A*01, Mamu-B*08, and Mamu-B*17 were excluded. The presence of Mamu-B*08 or Mamu-B*17 alone has been correlated to a reduction in plasma viremia [36, 62]. Although the effect is much weaker, Mamu-A*01 expression has also been linked to control of viral replication [13, 63]. Animals were cared for according to the regulations and guidelines of the University of Wisconsin Institutional Animal Care and Use Committee.

Viral suppression assay. Peripheral blood mononuclear cells (PBMC) were obtained on day -2 using Ficoll-Paque PLUS (Amersham Biosciences, Uppsala, Sweden) density centrifugation. Targets were generated by depleting CD8+ cells from PBMC using MACS nonhuman primate CD8 MicroBead Kit (Miltenyi Biotec), according to the manufacturer's instructions. The targets were activated by incubation with Concanavalin A (5 μg/mL) for 18 to 24 hours. Cells were cultured in R15-50 (RPMI 1640 containing 15% fetal calf serum and 50 U/mL 1L-2) throughout all parts of the assay. On day 0, CD3+ CD8+ effectors were obtained from control and vaccinated animals using a MACS Nonhuman primate CD8+ T-cell isolation kit (Miltenyi Biotec). These cells were consistently >90% pure. Sucrose-purified SIVmac239 or SIVsmE660 (10⁹ vRNA copies) were used to infect targets by spinoculation [48]. Approximately 5.0×10⁵ target cells were incubated per well in 96-well plates with the concentrated viruses in a final volume of 100 μl. Then, the plates were centrifuged at 1,200×g for 2 h at 25° C., and the cells were washed twice with medium. The superinfected targets were mixed with uninfected targets in a 1:10 ratio to seed the infection. Approximately 1.0×10⁵ cells of this mixture of infected targets were co-cultured with autologous effectors in 24-well plates for 7 days. Effector/target ratios of 0.1:1, 0.5:1, and 1:1 were utilized. On days 3 and 5, 0.5 ml of supernatant was removed from each well and replaced with fresh R15-50. On day 7, the cells were harvested and stained with fluorescently labeled antibodies specific to CD3, CD4, and CD8 (BD Biosciences). Then, the cells were permeabilized with Fix and Perm (Caltag, Burlingame, Calif.) and stained with FITC-conjugated antibodies specific to SIV Gag p27 (NIH AIDS Research and Reference Reagent Program, Germantown, Md.). The samples were then washed twice and run on a BD-LSRII flow cytometer (BD Biosciences) using FACSDiva software (BD Biosciences). Data were analyzed using FlowJo for Macintosh (Tree star). Suppression of viral replication was quantified based on the percent reduction of SIV Gag+ cells using the following formula: [(% of SIV Gag+ targets in the absence of effectors)−(% of SIV Gag+ targets at E:T ratios)/(% of SIV Gag+ targets in the absence of effectors)]×100.

Vaccination and challenge. A complete description of these items was published as part of a prior study [59]. Briefly, a DNA/Ad5 prime/boost regimen was utilized to immunize animals with codon-optimized sequences encoding the following proteins from SIVmac239: Gag, Tat, Rev, Nef, Pol, Vif, Vpr, and Vpx. Thirty-seven weeks after vaccination was completed, animals in the vaccinated and control groups were challenged intrarectally with up to 5 challenges of SIVsmE660 (800 50% tissue culture infective doses [TCID₅₀]; 1.2×10⁷SIV RNA copy eq). If not infected, they were subsequently challenged with a 5 times higher dose (4,000 TCID₅₀ or 6.0×10⁷ SIV RNA copy eq.) until infected. Repeated low-dose mucosal challenges [43] with SIVsmE660 were utilized to better approximate clinical exposures to HIV. Animals were considered to be infected after at least two consecutive positive viral load determinations, after which they were no longer challenged.

Viral load determination and absolute CD4+ T-cell counts. A complete description of these items was published as part of a prior study [59]. Levels of circulating plasma virus were determined using a previously described quantitative reverse transcription-PCR assay [51]. Virus concentrations were determined by interpolation onto a standard curve of in vitro-transcribed RNA standards in serial 10-fold dilutions using the Lightcycler 2.0 (software version 4; Roche). To get absolute CD4+ counts, whole PBMC were stained with fluorescently labeled antibodies specific for CD3 Alexa 700 (BD Pharmingen, San Diego, Calif.), CD4 PerCP (BD Pharmingen, San Diego, Calif.), CD8 Pacific Blue (BD Pharmingen), CD95 fluorescein isothiocyanate (BD Pharmingen), CD28 phycoerythrin (Becton Dickinson, San Jose, Calif.), and beta7 integrin allophycocyanin (Becton Dickinson). In brief, 500,000 PBMCs were incubated with these antibodies for 30 min at room temperature. The samples were then washed twice, fixed with paraformaldehyde, and run on a BD-LSR-II flow cytometer (Becton Dickinson) using FACSDiva software (BD Biosciences). Data were analyzed using FlowJo software. Absolute counts were calculated by multiplying the frequency of CD3⁺/CD4⁺ T cells within the lymphocyte gate by the lymphocyte counts per microliter of blood obtained from matching complete blood counts.

IFN-γ ELISPOT assays. A complete description of this technique has been published as part of a prior study [59]. Briefly, whole or CD8-depleted PBMC were used in enzyme-linked immunospot (ELISPOT) assays for the detection of IFN-γ-secreting cells. To determine positivity, the number of spots in duplicate wells (10⁵ per well) was averaged, and the background was subtracted. This resultant number is required to be greater than five spots (50 spot-forming cells [SFC]/million PBMCs) and also greater than 2 standard deviations over the background. Positive results were multiplied by 10 to get SFCs/million cells.

Statistical analyses. To compare vaccinees and control animals in the viral suppression assays, the normality and homogeneity of variances (homoscedasticity) of the suppression values were tested using the Kolmogorov-Smirnov and Levene's tests, respectively. If the variances in a given E:T ratio were heterogeneous, natural log-transformed values were used to correct for this and retesting for normality and homoscedasticity of residuals was performed. The two groups at each E:T ratio were compared using either the Wilcoxon signed-rank test or the Student t-test, as appropriate. For plasma viral concentrations, the values were log-transformed to reduce right-skewness and heteroscedasticity. In the comparisons of T-cell parameters and markers of disease progression (viral loads and absolute CD4+ T-cell counts), correlation was measured using the Kendall's tau and Spearman correlation tests. All significance tests were two-tailed.

Results

Vaccine-induced CD8+ T-cells suppress replication of a homologous virus in vitro. The efficacy of a DNA/Ad5 T-cell based vaccine encoding all SIVmac239 proteins except for Env was tested recently [59]. In order to define correlates of protection, here, a viral suppression assay (VSA) was developed in order to directly measure the antiviral activity of CD8+ T-cells in vitro [37]. This assay may be relevant for screening CD8+ T-cell responses associated with control of HIV infection [47, 55]. Thus, the ability of pre-challenge vaccine-induced CD8+ T-cells to suppress viral replication in vitro would be hypothesized to predict the outcome of the vaccine trial.

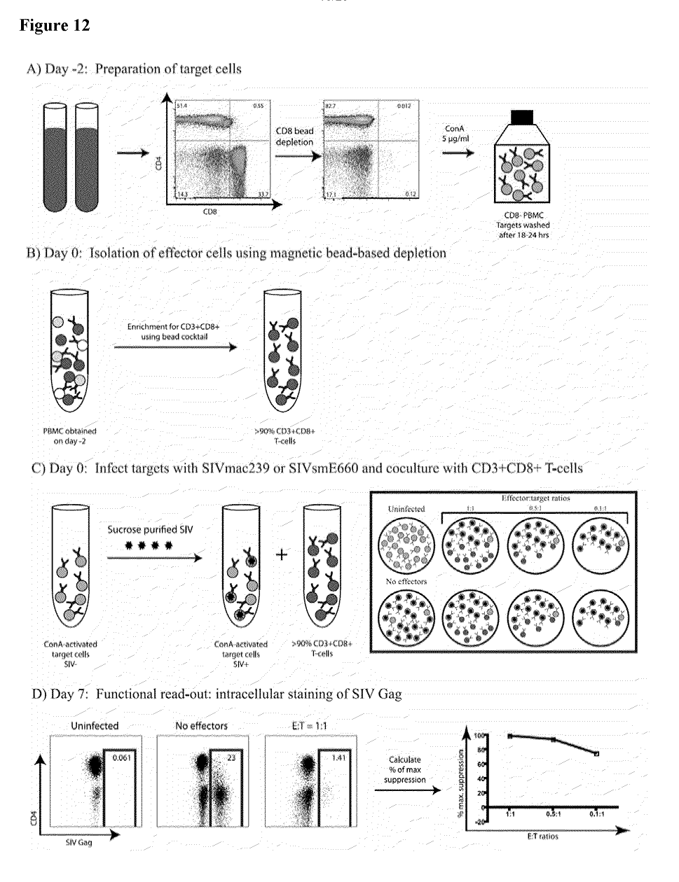

Sixteen weeks after the Ad5 boost, the suppressive ability of CD8+ T-cells from both control and vaccinated animals was screened. To setup the VSAs, fresh PBMC on day -2 were utilized (FIG. 12). Then, CD8+ cells were depleted from half of the PBMC to generate target cells, which then were incubated overnight with Concanavalin A (Con-A) (5 μg/ml) to stimulate them (FIG. 12A). Unstimulated PBMC were kept until day 0, at which time bulk CD8+ T-cells were isolated to use as effectors (FIG. 12B). On day 0, the ConA-activated targets were infected with SIVmac239 and cultured with the autologous unstimulated CD8+ T-cells. Three effector-to-target ratios were studied (0.1:1, 0.5:1, and 1:1 (FIG. 12C)). As described previously [37], suppression of viral replication after 7 days was measured by comparing the percentage of SIV-Gag+ cells in the presence or absence of effectors (FIG. 12D).

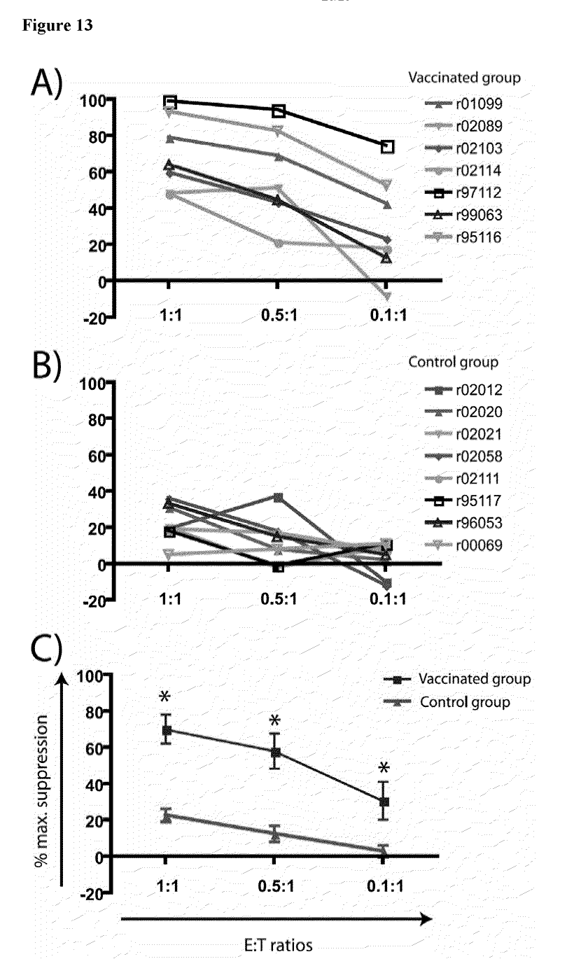

CD8+ T-cells from all vaccinees suppressed SIVmac239 replication with varying levels of efficiency (Table 9 and FIG. 13A). Animal r97112 (open black squares), for instance, had the best performance in the VSAs with suppression values higher than 75% at all E:Ts (Table 9 and FIG. 13A). However, the CD8+ T-cells from animals r02114 (closed circles) and r02089 (inverted triangles) only suppressed viral replication by 60% or less, even at the highest E:T ratio (Table 9 and FIG. 13A). Control unvaccinated animals had low levels of suppression at the highest E:T ratio (Table 9 and FIG. 13B). Importantly, the average suppression among vaccinees was significantly higher than that of control animals at all E:T ratios (FIG. 13C). Together, these results indicate that vaccine-induced CD8+ T-cells can suppress the replication of SIVmac239, a virus whose sequence is identical to the one used in the vaccine formulation.

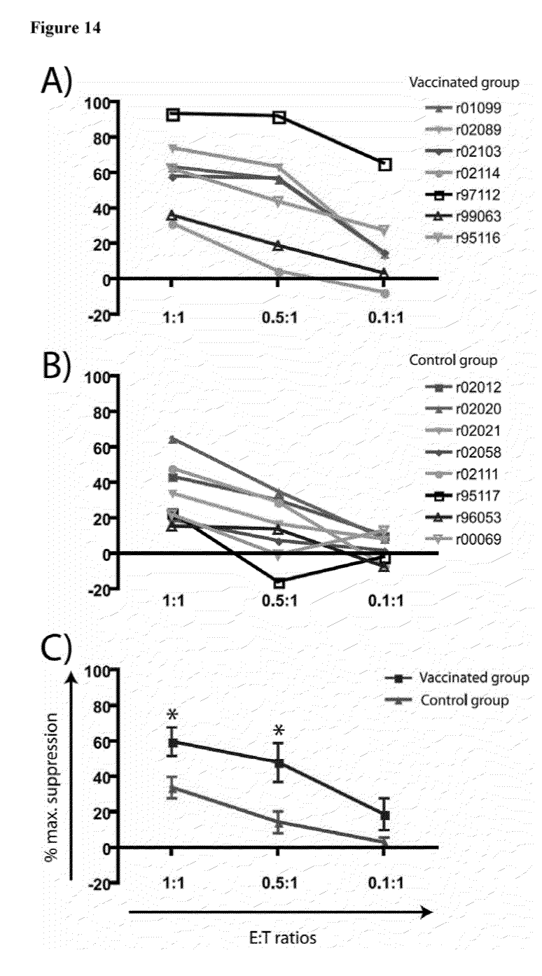

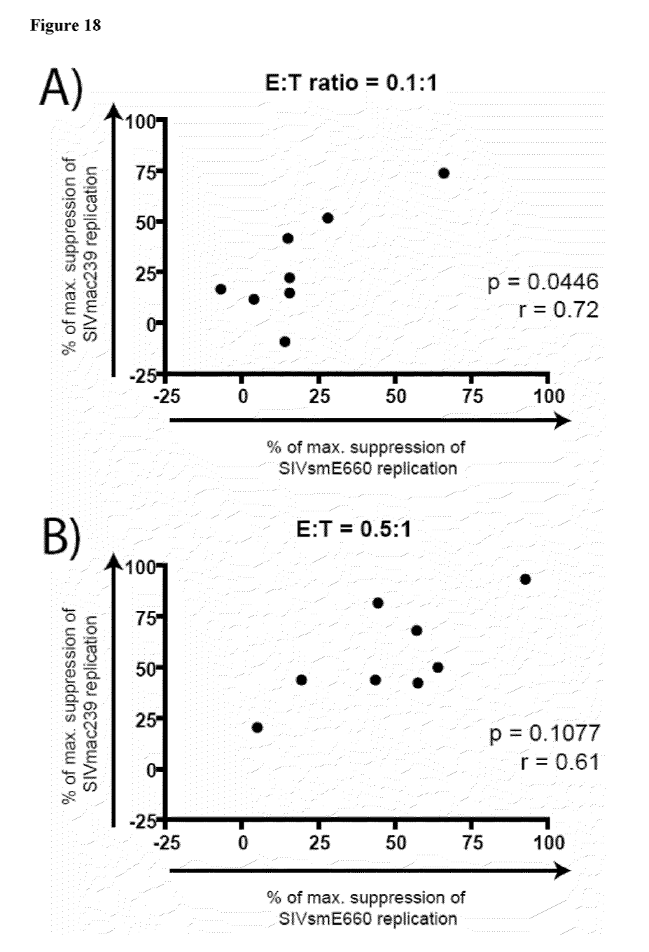

Vaccine-induced CD8+ T-cells suppress replication of a heterologous virus in vitro. An effective AIDS vaccine must generate immunity against the tremendously diverse population of circulating viruses. Indeed, HIV isolates within a single clade can vary in sequence as much as 10-20%, depending on the viral gene [21]. Using this rationale, we assessed whether the antiviral activity of CD8+ T-cells elicited against SIVmac239 by vaccination would also be effective against a different strain of SIV. VSAs were performed using targets infected with SIVsmE660, a biological isolate that differs from SIVmac239 by approximately 15% of its amino acids [59]. Compared to the control group, CD8+ T-cells from vaccinees suppressed the replication of SIVsmE660 in vitro at E:T ratios of 1:1 and 0.5:1 (Table 10 and FIGS. 14A & C). However, suppression at the E:T ratio of 0.1:1 was not significantly different between the vaccinated and control groups (p>0.05) (FIG. 14C). Similar to the results with SIVmac239, CD8+ T-cells from r97112 had the best performance in the VSAs, while r99063 and r02114 were the worst suppressors (Table 10 and FIG. 14A). Overall, suppression of SIVsmE660 replication among vaccinees tended to match that of SIVmac239 in the VSAs, although this association only reached statistical significance at the E:T ratio of 0.1:1 (p=0.0446; r=0.72) (FIG. 18). CD8+ T-cells from control animals also suppressed SIVsmE660 replication at the highest E:T ratio (Table 10 and FIG. 14B). In sum, CD8+ T-cells elicited by vaccination exerted antiviral activity against a heterologous “swarm” virus in vitro. Nonetheless, this suppressive ability was lost when vaccine-induced effectors were diluted to a 01:1 E:T ratio.

In vitro suppression of SIV replication did not predict vaccine efficacy. Thirty-seven weeks after vaccination, the animals were challenged intra-rectally (is.) with repeated low doses of SIVsmE660 [59]. The vaccine did not confer protection against acquisition of SIV infection, since both vaccinees and control animals became infected after an average of 4 challenges [59]. However, it is possible that one vaccinee, r00061, did not become productively infected [59]. Low levels of viremia were detected in this animal at only three timepoints. Nevertheless, two independent laboratories could not amplify virus from shipped frozen samples. No evidence of viral replication was found even after depleting this animal's CD8+ cells in vivo with a monoclonal antibody—a treatment that normally causes viral rebound even in animals controlling SIV infection. Therefore, r00061 was excluded from all correlative analyses.

After infection, five of eight vaccinees controlled viral replication to less than 80,000 vRNA copies/ml in the acute phase and to less than 100 vRNA copies/ml in the chronic phase (Table 11) [59]. Compared to control animals, this represented a reduction of 1.9 and 2.6 logs in peak and setpoint viremias, respectively [59]. We, therefore, sought to assess the cellular factors associated with the successful outcome of this vaccine trial.

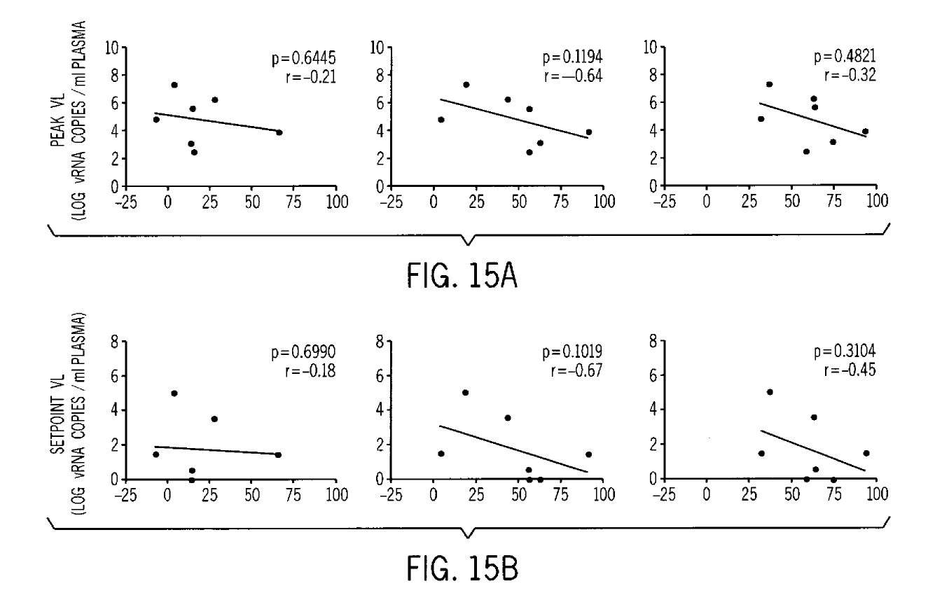

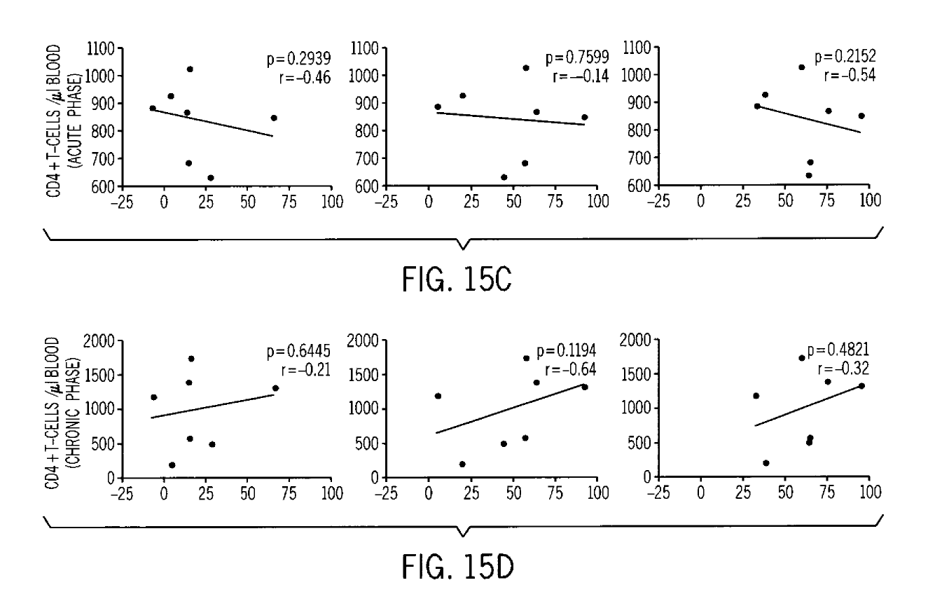

CD8+ T-cell-mediated suppression of viral replication has been suggested to reflect immune control of HIV replication in vivo [47, 55]. To test this ability as a surrogate marker for CD8+ T-cell efficacy in the vaccine trial, the percentages of maximum suppression of SIVsmE660 replication were compared (Table 10 and FIG. 14A) with prognostic markers of AIDS progression. The latter markers included peak and setpoint viral loads, and CD4+ lymphocyte counts measured in both the acute (weeks 2-3 post infection) and chronic (week 24 post infection) phases of infection [33, 45, 46]. Correlation between the above variables was measured using the Spearman rank correlation test and the results were plotted as scatterplots (FIG. 15). Of note, the Kendall's tau correlation test also was utilized to analyze these data and similar results were obtained (data not shown).

The ability of vaccine-induced CD8+ T-cells to suppress replication of the heterologous isolate SIVsmE660 in vitro did not predict vaccine efficacy (FIG. 15). When compared to the animals' viral loads in both the acute (FIG. 15A) and chronic phases (FIG. 15B), the VSA values at all 3 E:T ratios yielded no statistically significant correlations. Likewise, in vitro suppression did not associate with the absolute CD4+ T-cell counts obtained in either the acute or chronic phases (FIGS. 15C and D). Similarly, no correlation between the ability to suppress replication of the homologous clone SIVmac239 and these markers of disease progression was found (data not shown). Therefore, although vaccine-induced CD8+ T-cells suppressed the replication of SIVsmE660 and SIVmac239 in vitro, this ability was not predictive of vaccine efficacy after a heterologous challenge.

Correlations between the magnitude of vaccine-induced responses and markers of disease progression. Since the VSA values did not predict the outcome of the heterologous challenge, we analyzed pre- and post-challenge T-cell responses and attempted to correlate them with outcome. Pre-challenge responses were measured on day 14 after the Ad5 boost by carrying out whole proteome ELISPOT with PBMC from all of the vaccinees. We used 83 pools consisting of 10 15-mers (overlapping by 11 amino acids) spanning the nine SIVmac239 open reading frames. In addition, we mapped as many epitopes as possible to single 15-mers [59]. Because we did not have sufficient numbers of cells to measure the extent of vaccine-elicited SIV-specific CD4+ T-cell responses, all ELISPOT data generated before challenge included both CD4+ and CD8+ T-cell responses.

As shown in Table 12, the vaccinees mounted high-frequency T-cell responses against 11-34 epitopes in the vaccine [59]. Unexpectedly, all animals developed at least one response against Env even though the vaccine did not encode this protein (Table 12). Further analyses demonstrated that these Env-specific responses were derived from alternate reading frames (ARFs) in the Rev-encoding plasmid and Ad5 vector [38]. Of note, no Env-specific antibodies engendered by the vaccine were detected. Importantly, none of these ARF-derived responses against Env correlated with any markers of disease progression in this study (data not shown).

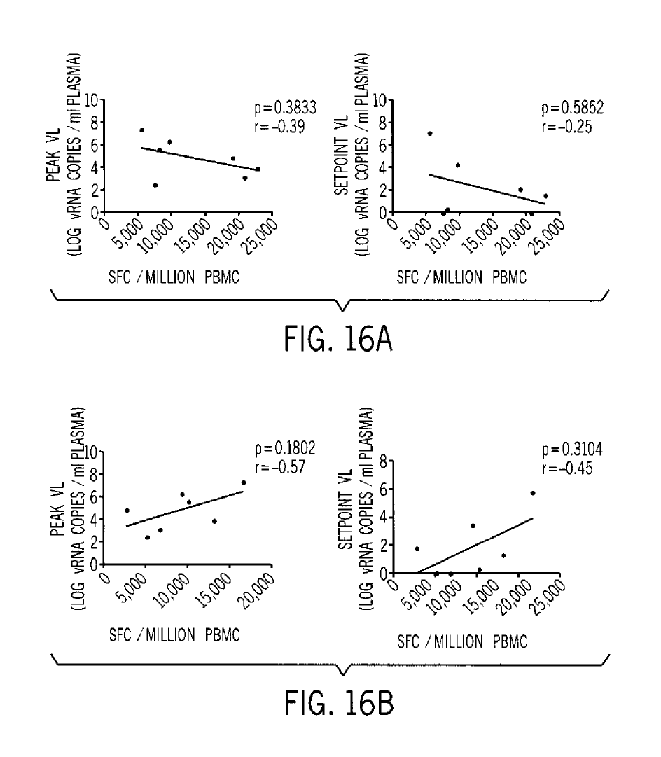

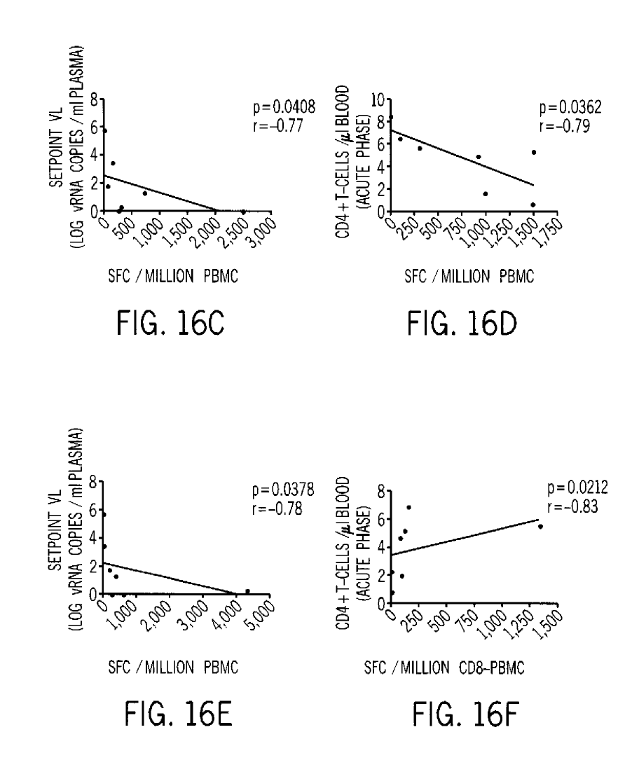

The analysis revealed that there was no statistically significant correlation between the total magnitude of pre-challenge responses and either peak or setpoint viral loads (FIG. 16A). Nonetheless, vaccine-induced responses directed against Vpr were associated with delayed disease progression. An inverse correlation between the magnitude of Vpr-specific T-cells and setpoint viremia was observed (p=0.0408; r=−0.77) (FIG. 16C). However, the frequency of responses against this protein varied significantly among vaccinees. IFN-γ+ T-cells specific for Vpr ranged from 0 to 2,493 SFC/10⁶PBMC in r99063 and r02089, respectively, compared to a median of 255 SFC/10⁶ PBMC (Table 12).

Surprisingly, the magnitude of Rev-directed responses was associated with diminished CD4+ T-cell counts in the acute phase (p=0.0362; r=−0.79) (FIG. 16D). Responses against this protein had a median of 920 SFC/10⁶PBMC, and ranged from no responses in r02103 to 1,510 SFC/10⁶PBMC in r02089 (Table 12). Despite this negative association with CD4+ T-cell counts, we did not find any relationship between the frequency of Rev-specific T-cells and either peak (p=0.8790; r=0.07) or setpoint (p=0.7578; r=−0.14) viral loads.

The anamnestic expansion of vaccine-induced responses after challenge with SIVsmE660 also was studied by performing IFN-γ ELISPOT at approximately two weeks after infection, before the expansion of de novo responses against SIVsmE660. Only responses observed after vaccination were analyzed. Interestingly, only 50% of the vaccine-induced responses expanded after challenge, likely due to the difference in sequence between the vaccine and challenge virus. The magnitude of the entire anamnestic post-challenge T-cell response did not correlate with the peak or setpoint viral loads (FIG. 16B). However, the magnitude of recall responses directed to Nef inversely correlated with setpoint viremia (p=0.0378; r=−0.78) (FIG. 16E). Given a median of 255 SFC/10⁶PBMC, the anamnestic responses against this protein were highly variable among vaccinees (Table 13). The frequency of Nef-specific IFN-γ+ T-cells ranged from no responses in r95116 and r99063 to 4,286 SFC/10⁶PBMC in r01099 (Table 13).

In order to evaluate the expansion of the virus-specific CD4+ T-cell compartment after challenge, CD8-depleted ELISPOT was performed. IFN-γ+ responses against Pol were found to be positively associated with the absolute CD4+ T-cell counts in the chronic phase (p=0.0212; r=0.83) (FIG. 16F). However, similar to the Vpr and Nef correlations above, the magnitude of Pol-specific CD4+ T-cell responses varied significantly. Its median was 90 SFC/10⁶PBMC, and the frequency of responses ranged from no responses in r01099 and r99063 to 1,342 SFC/10⁶PBMC in r02089 (Table 13).

We also searched for correlations among the frequency of pre- and post-challenge responses directed against combinations of structural, accessory, and regulatory proteins and the outcome of challenge (Table 14). In agreement with the above results, T-cell responses targeting combinations of the accessory proteins Vpr, Nef, and Vpx were associated with delayed disease progression (Table 14).

In order to maintain the family-wise Type I error rate no higher than alpha, the Bonferroni correction was applied to the analyses. Although the associations described above for the magnitude of immune responses directed against Vpr, Rev, and Pol were significant at the uncorrected nominal 0.05 level, they did not meet the statistical criterion established by the Bonferroni adjustment (0.0008).

In summary, a negative association between the frequency of vaccine-induced Rev-specific responses and lower CD4+ T-cell counts in the acute phase was observed. In addition, the magnitude of responses against Vpr, Nef, and Pol were associated with delayed disease progression. However, these correlations might not reflect the true role of these responses in SIV infection due to high animal-to-animal variability.

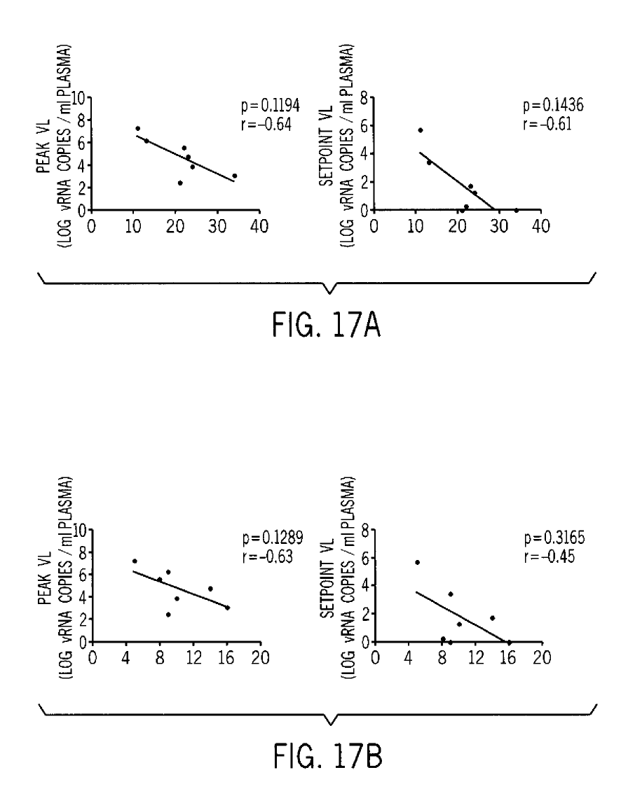

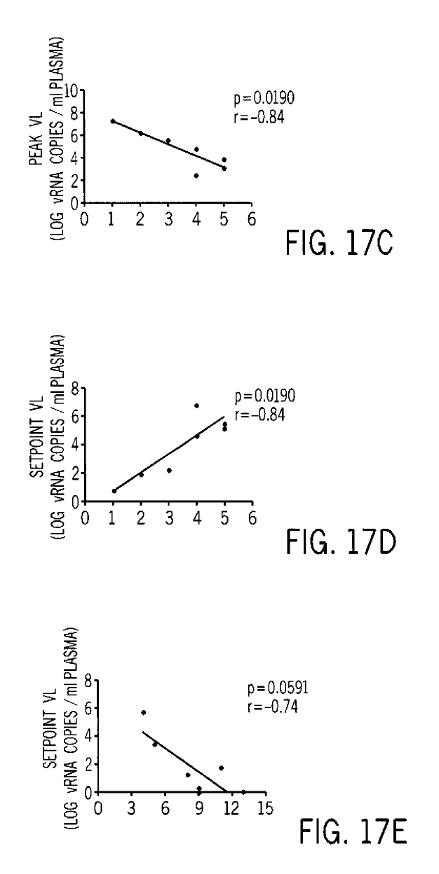

Correlations between the breadth of vaccine-induced responses and markers of disease progression. We also investigated whether the breadth of the vaccine-induced T-cell responses was associated with any markers of disease progression. There was no statistically significant correlation between the total number of epitopes recognized by each vaccinee and either peak or setpoint viral loads (FIG. 17A and B). However, the number of Vif epitopes was associated with two markers of delayed disease progression. Animals that targeted more epitopes in Vif had reduced peak viremia (p=0.0190; r=−0.84) (FIG. 17C) and higher CD4+ T-cell counts in the chronic phase (p=0.0190; r=0.84) (FIG. 17D). Of note, these associations were not significant at the Bonferroni-corrected level (0.0008). Importantly, all vaccinees mounted Vif-specific responses, ranging from 1 epitope in r99063 to 5 epitopes in both r97112 and r02089 (Table 12).

Broad epitope recognition in Gag was also associated with control of SIV replication (FIG. 17E). Increasing the breadth of pre-challenge responses against this protein correlated with lower setpoint viremia (p=0.0591; r=−0.74) (FIG. 17E). Despite the borderline p-value, this association may be biologically relevant given several other studies demonstrating a protective role for broad Gag-specific responses in the control of chronic HIV [17, 31] and SIV [53] infections. Additionally, the number of pre-challenge Gag epitopes has recently been correlated with control of chronic phase viral replication in vaccinated macaques that were challenged with SIVmac251 [35].

Correlation analyses with responses targeting combinations of viral proteins yielded several significant associations. The total number of epitopes recognized in Gag and Pol after vaccination was associated with control of viral replication in the chronic phase (Table 15). Furthermore, the breadth of responses directed against all accessory proteins was linked to controlled viremia and preservation of CD4+ T-cell counts in the chronic phase. Notably, the number of epitopes in Vif, combined to the number of T-cell responses targeting epitopes in either Vpr or Vpx, correlated with delayed disease progression even at the more strict Bonferroni corrected level (p≦0.0008) (Table 15).

Together, these findings suggest that increasing the breadth of cellular responses against Vif and Gag may contribute to the control of SIV replication. Thus, vaccines that engender broad Gag- and Vif-specific T-cells might be useful in controlling AIDS virus replication.

### Discussion

In the present study, we defined aspects of vaccine-induced cellular responses that correlated with the control of viral replication after a heterologous challenge with the pathogenic swarm virus SIVsmE660 [59]. While CD8+ T-cells from vaccinated animals suppressed replication of both homologous and heterologous viruses in vitro, this ability did not predict viral control in vivo after challenge. However, broad immune responses against Vif and Gag, and high frequency T-cell responses to Vpr, Nef, and Pol correlated with containment of SIVsmE660 infection.

The notion that broad cellular responses to certain viral proteins may impact control of immunodeficiency virus replication has been proposed before [31]. In this study, broad pre-challenge vaccine-induced responses against Gag and Vif were associated with markers of delayed disease progression. The number of epitopes in Vif, in particular, correlated with higher CD4+ T-cell counts in the chronic phase and lower peak viral loads. Achieving the latter result would be greatly desired for a T-cell based AIDS vaccine, since limiting viral replication in the acute phase would alleviate the damage to the gut CD4+ T-cell memory compartment that occurs in this stage of infection [34, 42]. To the present inventors' knowledge, this is the first report to implicate vaccine-induced cellular immune responses against Vif in the containment of SIV infection. The pre-challenge epitope breadth in Gag also correlated with a marker of delayed disease progression. Vaccinees that mounted broad responses against this protein had lower setpoint viral loads, although the p-value for this correlation (p=0.0591) was slightly greater than the 0.05 threshold of statistical significance. However, this finding agrees with previous studies showing that Gag-specific responses elicited by vaccination were associated with control of chronic phase viral replication [25, 26, 35]. Thus, the results indicate that cellular immune responses targeting Gag and Vif might be particularly efficient at containing viral replication after a heterologous SIV challenge.

Several studies have attempted to identify associations between the magnitude of HIV-1-specific responses and markers of disease progression [9, 17, 23, 39]. These studies have generated variable results and no particular pattern of responses is consistently associated with delayed disease progression. In the cohort of vaccinated Indian rhesus macaques, lower setpoint viral loads correlated with two aspects of vaccine-induced immune responses: the frequency of pre-challenge responses to Vpr, and the magnitude of post-challenge Nef-specific responses. The latter finding is consistent with a recent report showing that the magnitude of post-challenge responses to Nef inversely correlated with setpoint viral loads in vaccinated macaques challenged with SHIV89.6P [32]. Furthermore, Vpr has been suggested as a preferential target of CTLs in natural HIV-1 infection although its role on the control of viral replication is unknown [3]. In this study, the anamnestic CD4+ T-cell response to Pol also correlated with the preservation of CD4+ T-cell counts in the chronic phase. This result agrees with the notion that virus-specific T-helper responses actively participate in the control of immunodeficiency virus infection [25, 50, 54]. Nonetheless, the animal-to-animal variability in the frequency of IFN-γ+ T-cells against Vpr, Nef, and Pol among vaccinees was high, which makes it difficult to interpret these results. With a sample size limited to seven vaccinees, the presence of outliers in these correlations may have affected the predictive value of these comparisons. Therefore, more studies will be required to determine the precise role of vaccine-induced responses to Vpr, Nef, and Pol in the control of SIV infection.

Our analyses also indicate that the pre-challenge magnitude of Rev-specific IFN-γ-secreting T-cells inversely correlated with absolute CD4+ lymphocyte counts in the acute phase. However, RNA viral load has been suggested to carry more prognostic value than CD4+ lymphocyte counts early in the infection [33]. Since no correlation between the frequency of T-cells targeting Rev and either peak or setpoint viral loads was observed, it is unlikely that cellular responses to this protein accelerated the progression to AIDS.

Measuring the ability of CD8+ T-cells to suppress viral replication has been proposed as a relevant readout for immune control of HIV-1 [18]. Recently, two groups have shown that CD8+ T-cells from elite controllers suppress HIV-1 replication more efficiently than those of progressors [47, 55]. These findings are encouraging in that they begin to shed light on the mechanism of elite control and thus may be useful for the screening of efficient HIV-1-specific responses after vaccination. Using this rationale, we explored whether the ability of pre-challenge vaccine-elicited CD8+ T-cells to suppress SIV replication in vitro would predict how well vaccinees controlled viral replication after challenge. CD8+ T-cells from vaccinated animals suppressed the replication of both SIVmac239, the virus whose sequence is identical to the one used in the vaccine, and SIVsmE660, the heterologous virus used for the challenge. Although suppression among vaccinees was heterogeneous, this measurement did not correlate with any prognostic markers of progression to AIDS. Therefore, suppression of viral replication in vitro by vaccine-elicited CD8+ T-cells did not predict the efficacy of a T-cell based vaccine against SIV.

Two factors likely interfered with the comparisons between the VSA values and markers of disease progression. The first was our sample size (n=7 vaccinees), which might not have been large enough for the detection of statistically significant differences, especially given the complexity of CTL-mediated antiviral immunity [4, 60]. Characteristics of the swarm virus SIVsmE660 and the low-dose challenge route used in this vaccine trial may also have hampered our ability to correlate virus suppression and vaccine challenge outcome [59]. Recent studies have shown that productive HIV-1 infection across a mucosal surface is initiated by one or a few viral quasispecies [29]. To mimic these human HIV-1 exposures, the animals were challenged intra-rectally with repeated low-doses of SIVsmE660 [30, 59]. By using a titrated inoculum, both control and vaccinated animals became infected by one or a few SIVsmE660 viral variants [59]. By contrast, the targets used in the VSAs were exposed to the entire diversity of viral variants present in our SIVsmE660 stock, and likely at a higher MOI. Thus, we likely measured in vitro suppression of a multitude of quasispecies whose replicative fitness may not directly compare to those of the few variants that established infection in the animals.

The lack of significant correlations between in vitro suppression of viral replication and the outcome of challenge with SIVsmE660 may also be explained by the use of blood-derived CD8+ T-cells in the VSAs. Evaluating cellular immune responses from peripheral blood is convenient because of the ease in obtaining samples from this compartment. However, circulating T-cells might not be functionally equivalent to those present in mucosa-associated lymphoid tissues, where viral replication is concentrated [5, 6, 19, 24, 40]. In elite controllers, for example, mucosal CD8+ T-cell responses to HIV-1 have been shown to be more “polyfunctional” than those measured in blood [19]. Furthermore, the presence of virus-specific CD8+ T-cells in colonic lamina propria, but not in blood, has been correlated with delayed disease progression in vaccinated macaques challenged intra-rectally with SHIV-ku2 [6]. Therefore, assays that measure the antiviral function of mucosae-residing CD8+ T-cells might provide a more accurate approximation of the host cellular responses against the virus.

In summary, the goal of the present study was to identify the correlates of protection in vaccinated macaques that controlled viral replication after a heterologous SIV challenge [59]. The ability of vaccine-induced CD8+ T-cells to suppress viral replication in vitro did not predict viral containment in vivo. Nevertheless, broad cellular immune responses directed to Gag and Vif predicted the efficacy of a T-cell based vaccine against a heterologous SIV challenge. This knowledge may provide a rationale for the selection of immunogens that will be included in future T-cell based AIDS vaccines.

### References for Example 2

1. Addo, M. M., X. G. Yu, A. Rathod, D. Cohen, R. L. Eldridge, D. Strick, M. N. Johnston, C. Corcoran, A. G. Wurcel, C. A. Fitzpatrick, M. E. Feeney, W. R. Rodriguez, N. Basgoz, R. Draenert, D. R. Stone, C. Brander, P. J. Goulder, E. S. Rosenberg, M. Altfeld, and B. D. Walker. 2003. Comprehensive epitope analysis of human immunodeficiency virus type 1 (HIV-1)-specific T-cell responses directed against the entire expressed HIV-1 genome demonstrate broadly directed responses, but no correlation to viral load. J Virol 77:2081-2092.

2. Allen, T. M., M. Altfeld, S. C. Geer, E. T. Kalife, C. Moore, K. M. O'sullivan, I. Desouza, M. E. Feeney, R. L. Eldridge, E. L. Maier, D. E. Kaufmann, M. P. Lahaie, L. Reyor, G. Tanzi, M. N. Johnston, C. Brander, R. Draenert, J. K. Rockstroh, H. Jessen, E. S. Rosenberg, S. A. Mallal, and B. D. Walker. 2005. Selective escape from CD8+ T-cell responses represents a major driving force of human immunodeficiency virus type 1 (HIV-1) sequence diversity and reveals constraints on HIV-1 evolution. J Virol 79:13239-13249.

3. Altfeld, M., M. M. Addo, R. L. Eldridge, X. G. Yu, S. Thomas, A. Khatri, D. Strick, M. N. Phillips, G. B. Cohen, S. A. Islam, S. A. Kalams, C. Brander, P. J. Goulder, E. S. Rosenberg, and B. D. Walker. 2001. Vpr is preferentially targeted by CTL during HIV-1 infection. J Immunol 167:2743-2752.

4. Appay, V., D. C. Douek, and D. A. Price. 2008. CD8+ T cell efficacy in vaccination and disease. Nat Med 14:623-628.

5. Belyakov, I. M., and J. D. Ahlers. 2008. Functional CD8+ CTLs in mucosal sites and HIV infection: moving forward toward a mucosal AIDS vaccine. Trends Immunol 29:574-585.

6. Belyakov, I. M., V. A. Kuznetsov, B. Kelsall, D. Klinman, M. Moniuszko, M. Lemon, P. D. Markham, R. Pal, J. D. Clements, M. G. Lewis, W. Strober, G. Franchini, and J. A. Berzofsky. 2006. Impact of vaccine-induced mucosal high-avidity CD8+ CTLs in delay of AIDS viral dissemination from mucosa. Blood 107:3258-3264.

7. Bennett, M. S., H. L. Ng, A. Ali, and O. O. Yang. 2008. Cross-clade detection of HIV-1-specific cytotoxic T lymphocytes does not reflect cross-clade antiviral activity. J Infect Dis 197:390-397.

8. Bennett, M. S., H. L. Ng, M. Dagarag, A. Ali, and O. O. Yang. 2007. Epitope-dependent avidity thresholds for cytotoxic T-lymphocyte clearance of virus-infected cells. J Virol 81:4973-4980.

9. Betts, M. R., D. R. Ambrozak, D. C. Douek, S. Bonhoeffer, J. M. Brenchley, J. P. Casazza, R. A. Koup, and L. J. Picker. 2001. Analysis of total human immunodeficiency virus (HIV)-specific CD4(+) and CD8(+) T-cell responses: relationship to viral load in untreated HIV infection. J Virol 75:11983-11991.

10. Betts, M. R., M. C. Nason, S. M. West, S. C. De Rosa, S. A. Miguelcs, J. Abraham, M. M. Lederman, J. M. Benito, P. A. Goepfert, M. Connors, M. Roederer, and R. A. Koup. 2006. HIV nonprogressors preferentially maintain highly functional HIV-specific CD8+ T cells. Blood 107:4781-4789.

11. Buchbinder, S. P., D. V. Mehrotra, A. Duerr, D. W. Fitzgerald, R. Mogg, D. Li, P. B. Gilbert, J. R. Lama, M. Marmor, C. Del Rio, M. J. McElrath, D. R. Casimiro, K. M. Gottesdiener, J. A. Chodakewitz, L. Corey, and M. N. Robertson. 2008. Efficacy assessment of a cell-mediated immunity HIV-1 vaccine (the Step Study): a double-blind, randomised, placebo-controlled, test-of-concept trial. Lancet 372:1881-1893.

12. Carrington, M., and S. J. O'Brien. 2003. The influence of HLA genotype on AIDS. Annu Rev Med 54:535-551.

13. Casimiro, D. R., F. Wang, W. A. Schleif, X. Liang, Z. Q. Zhang, T. W. Tobery, M. E. Davies, A. B. McDermott, D. H. O'Connor, A. Fridman, A. Bagchi, L. G. Tussey, A. J. Bett, A. C. Finnefrock, T. M. Fu, A. Tang, K. A. Wilson, M. Chen, H. C. Perry, G. J. Heidecker, D. C. Freed, A. Carella, K. S. Punt, K. J. Sykes, L. Huang, V. 1. Ausensi, M. Bachinsky, U. Sadasivan-Nair, D. I. Watkins, E. A. Emini, and J. W. Shiver. 2005. Attenuation of simian immunodeficiency virus SIVmac239 infection by prophylactic immunization with dna and recombinant adenoviral vaccine vectors expressing Gag. J Virol 79:15547-15555.

14. Chung, C., W. Lee, J. T. Loffredo, B. Burwitz, T. C. Friedrich, J. P. Giraldo Vela, G. Napoe, E. G. Rakasz, N. A. Wilson, D. B. Allison, and D. I. Watkins. 2007. Not all cytokine-producing CD8+ T cells suppress simian immunodeficiency virus replication. J Virol 81:1517-1523.

15. De Rosa, S. D., and M. J. McElrath. 2008. T cell responses generated by HIV vaccines in clinical trials. Curr Opin HIV AIDS 3:375-379.

16. Desrosiers, R. C. 2004. Prospects for an AIDS vaccine. Nat Med 10:221-223.

17. Edwards, B. H., A. Bansal, S. Sabbaj, J. Bakari, M. J. Mulligan, and P. A. Goepfert. 2002. Magnitude of functional CD8+ T-cell responses to the gag protein of human immunodeficiency virus type 1 correlates inversely with viral load in plasma. J Virol 76:2298-2305.

18. Fauce, S. R., O. O. Yang, and R. B. Effros. 2007. Autologous CD4/CD8 co-culture assay: a physiologically-relevant composite measure of CD8+ T lymphocyte function in HIV-infected persons. J Immunol Methods 327:75-81.

19. Ferre, A. L., P. W. Hunt, J. W. Critchfield, D. H. Young, M. M. Morris, J. C. Garcia, R. B. Pollard, H. F. J. Yee, J. N. Martin, S. G. Deeks, and B. L. Shacklett. 2009. Mucosal immune responses to HIV-1 in elite controllers: a potential correlate of immune control. Blood 113:3978-3989.

20. Friedrich, T. C., L. E. Valentine, L. J. Yant, E. G. Rakasz, S. M. Piaskowski, J. R. Furlott, K. L. Weisgrau, B. Burwitz, G. E. May, E. J. Leon, T. Soma, G. Napoe, S. V. r. Capuano, N. A. Wilson, and D. I. Watkins. 2007. Subdominant CD8+ T-cell responses are involved in durable control of AIDS virus replication. J Virol 81:3465-3476.

21. Gaschen, B., J. Taylor, K. Yusim, B. Foley, F. Gao, D. Lang, V. Novitsky, B. Haynes, B. H. Hahn, T. Bhattacharya, and B. Korber. 2002. Diversity considerations in HIV-1 vaccine selection. Science 296:2354-2360.

22. Goulder, P. J., and D. I. Watkins. 2008. Impact of MHC class I diversity on immune control of immunodeficiency virus replication. Nat Rev Immunol 8:619-630.

23. Gray, C. M., M. Mlotshwa, C. Riou, T. Mathebula, D. de Assis Rosa, T. Mashishi, C. Seoighe, N. Ngandu, F. van Loggerenberg, L. Morris, K. Mlisana, C. Williamson, and S. A. Karim. 2009. Human immunodeficiency virus-specific gamma interferon enzyme-linked immunospot assay responses targeting specific regions of the proteome during primary subtype C infection are poor predictors of the course of viremia and set point. J Virol 83:470-478.

24. Greene, J. M., J. J. Lhost, B. J. Burwitz, M. L. Budde, C. E. Macnair, M. K. Weiker, E. Gostick, T. C. Friedrich, K. W. Broman, D. A. Price, S. O'Connor, and D. H. O'Connor. 2010. Extra-lymphoid tissue-resident CD8+ T cells from SIVmac239{Delta}nef-vaccinated macaques suppress SIVmac239 replication ex vivo. J Virol doi:10.1128/JVI.02028-09.

25. Hel, Z., J. Nacsa, E. Tryniszewska, W. P. Tsai, R. W. Parks, D. C. Montefiori, B. K. Felber, J. Tartaglia, G. N. Pavlakis, and G. Franchini. 2002. Containment of simian immunodeficiency virus infection in vaccinated macaques: correlation with the magnitude of virus-specific pre- and postchallenge CD4+ and CD8+ T cell responses. J Immunol 169:4778-4787.

26. Hel, Z., W. P. Tsai, E. Tryniszewska, J. Nacsa, P. D. Markham, M. G. Lewis, G. N. Pavlakis, B. K. Felber, J. Tartaglia, and G. Franchini. 2006. Improved vaccine protection from simian AIDS by the addition of nonstructural simian immunodeficiency virus genes. J Immunol 176:85-96.

27. Jin, X., D. E. Bauer, S. E. Tuttleton, S. Lewin, A. Gettie, J. Blanchard, C. E. Irwin, J. T. Safrit, J. Mittler, L. Weinberger, L. G. Kostrikis, L. Zhang, A. S. Perelson, and D. D. Ho. 1999. Dramatic rise in plasma viremia after CD8(+) T cell depletion in simian immunodeficiency virus-infected macaques. J Exp Med 189:991-998.

28. Kaizu, M., G. J. Borchardt, C. E. Glidden, D. L. Fisk, J. T. Loffredo, D. I. Watkins, and W. M. Rehrauer. 2007. Molecular typing of major histocompatibility complex class I alleles in the Indian rhesus macaque which restrict Sly CD8+ T cell epitopes. Immunogenetics 59:693-703.

29. Keele, B. F., E. E. Giorgi, J. F. Salazar-Gonzalez, J. M. Decker, K. T. Pham, M. G. Salazar, C. Sun, T. Grayson, S. Wang, H. Li, X. Wei, C. Jiang, J. L. Kirchherr, F. Gao, J. A. Anderson, L. H. Ping, R. Swanstrom, G. D. Tomaras, W. A. Blattner, P. A. Goepfert, J. M. Kilby, M. S. Saag, E. L. Delwart, M. P. Busch, M. S. Cohen, D. C. Montefiori, B. F. Haynes, B. Gaschen, G. S. Athreya, H. Y. Lee, N. Wood, C. Seoighe, A. S. Perelson, T. Bhattacharya, B. T. Korber, B. H. Hahn, and G. M. Shaw. 2008. Identification and characterization of transmitted and early founder virus envelopes in primary HIV-1 infection. Proc Natl Acad Sci U S A 105:7552-7557.

30. Keele, B. F., H. Li, G. H. Learn, P. Hraber, E. E. Giorgi, T. Grayson, C. Sun, Y. Chen, W. W. Yeh, N. L. Letvin, J. R. Mascola, G. J. Nabel, B. F. Haynes, T. Bhattacharya, A. S. Perelson, B. T. Korber, B. H. Hahn, and G. M. Shaw. 2009. Low-dose rectal inoculation of rhesus macaques by SIVsmE660 or SIVmac251 recapitulates human mucosal infection by HIV-1. J Exp Med 206:1117-1134.

31. Kiepiela, P., K. Ngumbela, C. Thobakgale, D. Ramduth, I. Honeyborne, E. Moodley, S. Reddy, C. de Pierres, Z. Mncube, N. Mkhwanazi, K. Bishop, M. van der Stok, K. Nair, N. Khan, H. Crawford, R. Payne, A. Leslie, J. Prado, A. Prendergast, J. Frater, N. McCarthy, C. Brander, G. H. Learn, D. Nickle, C. Rousseau, H. Coovadia, J. I. Mullins, D. Heckerman, B. D. Walker, and P. Goulder. 2007. CD8+ T-cell responses to different HIV proteins have discordant associations with viral load. Nat Med 13:46-53.

32. Koopman, G., D. Mortier, S. Hofman, N. Mathy, M. Koutsoukos, P. Ertl, P. Overend, C. van Wely, L. L. Thomsen, B. Wahren, G. Voss, and J. L. Heeney. 2008. Immune-response profiles induced by human immunodeficiency virus type 1 vaccine DNA, protein or mixed-modality immunization: increased protection from pathogenic simian-human immunodeficiency virus viraemia with protein/DNA combination. J Gen Virol 89:540-553.

33. Korenromp, E. L., B. G. Williams, G. P. Schmid, and C. Dye. 2009. Clinical prognostic value of RNA viral load and CD4 cell counts during untreated HIV-1 infection—a quantitative review. PLoS One 4:e5950.

34. Li, Q., L. Duan, J. D. Estes, Z. M. Ma, T. Rourke, Y. Wang, C. Reilly, J. Carlis, C. J. Miller, and A. T. Haase. 2005. Peak SIV replication in resting memory CD4+ T cells depletes gut lamina propria CD4+ T cells. Nature 434:1148-1152.

35. Liu, J., K. L. O'Brien, D. M. Lynch, N. L. Simmons, A. La Porte, A. M. Riggs, P. Abbink, R. T. Coffey, L. E. Grandpre, M. S. Seaman, G. Landucci, D. N. Forthal, D. C. Montefiori, A. Carville, K. G. Mansfield, M. J. Havenga, M. G. Pau, J. Goudsmit, and D. H. Barouch. 2009. Immune control of an SIV challenge by a T-cell-based vaccine in rhesus monkeys. Nature 457:87-91.

36. Loffredo, J. T., J. Maxwell, Y. Qi, C. E. Glidden, G. J. Borchardt, T. Soma, A. T. Bean, D. R. Beal, N. A. Wilson, W. M. Rehrauer, J. D. Lifson, M. Carrington, and D. I. Watkins. 2007. Mamu-B*08-positive macaques control simian immunodeficiency virus replication. J Virol 81:8827-8832.

37. Loffredo, J. T., E. G. Rakasz, J. P. Giraldo, S. P. Spencer, K. K. Grafton, S. R. Martin, G. Napoe, L. J. Yant, N. A. Wilson, and D. I. Watkins. 2005. Tat(28-35)SL8-specific CD8+ T lymphocytes are more effective than Gag(181-189)CM9-specific CD8+ T lymphocytes at suppressing simian immunodeficiency virus replication in a functional in vitro assay. J Virol 79:14986-14991.

38. Maness, N. J., N. A. Wilson, J. S. Reed, S. M. Piaskowski, J. B. Sacha, A. D. Walsh, E. Thoryk, G. J. Heidecker, M. P. Citron, X. Liang, A. J. Bett, D. R. Casimiro, and D. I. Watkins. Robust, vaccine-induced CD8+ T lymphocyte response against an out of frame epitope. J Immunol 184:67-72.

39. Masemola, A., T. Mashishi, G. Khoury, P. Mohube, P. Mokgotho, E. Vardas, M. Colvin, L. Zijenah, D. Katzenstein, R. Musonda, S. Allen, N. Kumwenda, T. Taha, G. Gray, J. McIntyre, S. A. Karim, H. W. Sheppard, and C. M. Gray. 2004. Hierarchical targeting of subtype C human immunodeficiency virus type 1 proteins by CD8+ T cells: correlation with viral load. J Virol 78:3233-3243.

40. Masopust, D., V. Vezys, E. J. Wherry, D. L. Barber, and R. Ahmed. 2006. Cutting edge: gut microenvironment promotes differentiation of a unique memory CD8 T cell population. J Immunol 176:2079-2083.

41. Matano, T., R. Shibata, C. Siemon, M. Connors, H. C. Lane, and M. A. Martin. 1998. Administration of an anti-CD8 monoclonal antibody interferes with the clearance of chimeric simian/human immunodeficiency virus during primary infections of rhesus macaques. J Virol 72:164-169.

42. Mattapallil, J. J., D. C. Douek, B. Hill, Y. Nishimura, M. Martin, and M. Roederer. 2005. Massive infection and loss of memory CD4+ T cells in multiple tissues during acute SIV infection. Nature 434:1093-1097.

43. McDermott, A. B., J. Mitchen, S. Piaskowski, I. De Souza, L. J. Yant, J. Stephany, J. Furlott, and D. I. Watkins. 2004. Repeated low-dose mucosal simian immunodeficiency virus SIVmac239 challenge results in the same viral and immunological kinetics as high-dose challenge: a model for the evaluation of vaccine efficacy in nonhuman primates. J Virol 78:3140-3144.

44. McElrath, M. J., S. C. De Rosa, Z. Moodie, S. Dubey, L. Kierstead, H. Janes, O. D. Defawe, D. K. Carter, J. Hural, R. Akondy, S. P. Buchbinder, M. N. Robertson, D. V. Mehrotra, S. G. Self, L. Corey, J. W. Shiver, and D. R. Casimiro. 2008. HIV-1 vaccine-induced immunity in the test-of-concept Step Study: a case-cohort analysis. Lancet 372:1894-1905.

45. Mellors, J. W., A. Munoz, J. V. Giorgi, J. B. Margolick, C. J. Tassoni, P. Gupta, L. A. Kingsley, J. A. Todd, A. J. Saah, R. Detels, J. P. Phair, and C. R. J. Rinaldo. 1997. Plasma viral load and CD4+ lymphocytes as prognostic markers of HIV-1 infection. Ann Intern Med 126:946-954.

46. Mellors, J. W., C. R. J. Rinaldo, P. Gupta, R. M. White, J. A. Todd, and L. A. Kingsley. 1996. Prognosis in HIV-1 infection predicted by the quantity of virus in plasma. Science 272:1167-1170.

47. Migueles, S. A., C. M. Osborne, C. Royce, A. A. Compton, R. P. Joshi, K. A. Weeks, J. E. Rood, A. M. Berkley, J. B. Sacha, N. A. Cogliano-Shutta, M. Lloyd, G. Roby, R. Kwan, M. McLaughlin, S. Stallings, C. Rehm, M. A. O'Shea, J. Mican, B. Z. Packard, A. Komoriya, S. Palmer, A. P. Wiegand, F. Maldarelli, J. M. Coffin, J. W. Mellors, C. W. Hallahan, D. A. Follman, and M. Connors. 2008. Lytic granule loading of CD8+ T cells is required for HIV-infected cell elimination associated with immune control. Immunity 29:1009-1021.

48. O'Doherty, U., W. J. Swiggard, and M. H. Malim. 2000. Human immunodeficiency virus type 1 spinoculation enhances infection through virus binding. J Virol 74:10074-10080.

49. Peut, V., and S. J. Kent. 2007. Utility of human immunodeficiency virus type 1 envelope as a T-cell immunogen. J Virol 81:13125-13134.

50. Ramduth, D., C. L. Day, C. F. Thobakgale, N. P. Mkhwanazi, C. de Pierres, S. Reddy, M. van der Stok, Z. Mncube, K. Nair, E. S. Moodley, D. E. Kaufmann, H. Streeck, H. M. Coovadia, P. Kiepiela, P. J. Goulder, and B. D. Walker. 2009. Immunodominant HIV-1 Cd4+ T cell epitopes in chronic untreated clade C HIV-1 infection. PLoS One 4:e5013.

51. Reynolds, M. R., A. M. Weiler, K. L. Weisgrau, S. M. Piaskowski, J. R. Furlott, J. T. Weinfurter, M. Kaizu, T. Soma, E. J. Leon, C. MacNair, D. P. Leaman, M. B. Zwick, E. Gostick, S. K. Musani, D. A. Price, T. C. Friedrich, E. G. Rakasz, N. A. Wilson, A. B McDermott, R. Boyle, D. B. Allison, D. R. Burton, W. C. Koff, and D. I. Watkins. 2008. Macaques vaccinated with live-attenuated SIV control replication of heterologous virus. J Exp Med 205:2537-2550.

52. Rolland, M., D. Heckerman, W. Deng, C. M. Rousseau, H. Coovadia, K. Bishop, P. J. Goulder, B. D. Walker, C. Brander, and J. I. Mullins. 2008. Broad and Gag-biased HIV-1 epitope repertoires are associated with lower viral loads. PLoS One 3:e1424.

53. Sacha, J. B., C. Chung, E. G. Rakasz, S. P. Spencer, A. K. Jonas, A. T. Bean, W. Lee, B. J. Burwitz, J. J. Stephany, J. T. Loffredo, D. B. Allison, S. Adnan, A. Hoji, N. A. Wilson, T. C. Friedrich, J. D. Lifson, O. O. Yang, and D. I. Watkins. 2007. Gag-specific CD8+ T lymphocytes recognize infected cells before AIDS-virus integration and viral protein expression. J Immunol 178:2746-2754.

54. Sacha, J. B., J. P. Giraldo-Vela, M. B. Buechler, M. A. Martins, N. J. Maness, C. Chung, L. T. Wallace, E. J. Leon, T. C. Friedrich, N. A. Wilson, A. Hiraoka, and D. I. Watkins. 2009. Gag- and Nef-specific CD4+ T cells recognize and inhibit SIV replication in infected macrophages early after infection. Proc Natl Acad Sci U S A 106:9791-9796.

55. Saez-Cirion, A., C. Lacabaratz, O. Lambotte, P. Versmisse, A. Urrutia, F. Boufassa, F. Barre-Sinoussi, J. F. Delfraissy, M. Sinet, G. Pancino, and A. Venet. 2007. HIV controllers exhibit potent CD8 T cell capacity to suppress HIV infection ex vivo and peculiar cytotoxic T lymphocyte activation phenotype. Proc Natl Acad Sci U S A 104:6776-6781.

56. Seder, R. A., P. A. Darrah, and M. Roederer. 2008. T-cell quality in memory and protection: implications for vaccine design. Nat Rev Immunol 8:247-258.

57. Valentine, L. E., S. M. Piaskowski, E. G. Rakasz, N. L. Henry, N. A. Wilson, and D. I. Watkins. 2008. Recognition of escape variants in ELISPOT does not always predict CD8+ T-cell recognition of simian immunodeficiency virus-infected cells expressing the same variant sequences. J Virol 82:575-581.

58. Watkins, D. I. 2008. The hope for an HIV vaccine based on induction of CD8+ T lymphocytes: a review. Mem Inst Oswaldo Cruz 103:119-129.

59. Wilson, N. A., B. F. Keele, J. S. Reed, S. M. Piaskowski, C. E. MacNair, A. J. Bett, X. Liang, F. Wang, E. Thoryk, G. J. Heidecker, M. P. Citron, L. Huang, J. Lin, S. Vitelli, C. D. Ahn, M. Kaizu, N. J. Maness, M. R. Reynolds, T. C. Friedrich, J. T. Loffredo, E. G. Rakasz, S. Erickson, D. B. Allison, M. J. Piatak, J. D. Lifson, J. W. Shiver, D. R. Casimiro, G. M. Shaw, B. H. Hahn, and D. I. Watkins. 2009. Vaccine-induced cellular responses control simian immunodeficiency virus replication after heterologous challenge. J Virol 83:6508-6521.

60. Yang, O. O. 2003. Will we be able to ‘spot’ an effective HIV-1 vaccine? Trends Immunol 24:67-72.

61. Yang, O. O. 2008. Retracing our STEP towards a successful CTL-based HIV-1 vaccine. Vaccine 26:3138-3141.

62. Yant, L. J., T. C. Friedrich, R. C. Johnson, G. E. May, N. J. Maness, A. M. Enz, J. D. Lifson, D. H. O'Connor, M. Carrington, and D. I. Watkins. 2006. The high-frequency major histocompatibility complex class I allele Mamu-B*17 is associated with control of simian immunodeficiency virus SIVmac239 replication. J Virol 80:5074-5077.

63. Zhang, Z. Q., T. M. Fu, D. R. Casimiro, M. E. Davies, X. Liang, W. A. Schleif, L. Handt, L. Tussey, M. Chen, A. Tang, K. A. Wilson, W. L. Trigona, D. C. Freed, C. Y. Tan, M. Horton, E. A. Emini, and J. W. Shiver. 2002. Mamu-A*01 allele-mediated attenuation of disease progression in simian-human immunodeficiency virus infection. J Virol 76:12845-12854.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

TABLE 1
Vaccine-induced immune responses, measured at 14 days
after the Ad5 boost, are high frequency and broad.

| | | | | | | | | | | |
|---|---|---|---|---|---|---|---|---|---|---|
| Animal | | | | | | | | | | |
| ID | Gag | Nef | Tat | Rev | Vpr | Vpx | Vif | Env | Pol | Total |

Frequency of vaccine-induced immune responses (SFC/10⁶ PBMC).

| | | | | | | | | | | |
|---|---|---|---|---|---|---|---|---|---|---|
| r00061 | 3615 | | | 510 | | | 990 | 105 | 240 | 5460 |
| r02103 | 3258 | 785 | | | 255 | 60 | 2675 | 163 | 328 | 7523 |
| r02114 | 7183 | 385 | 1133 | 305 | 63 | | 6565 | 3425 | | 19058 |
| r01099 | 2745 | 680 | | 1000 | 303 | | 733 | 1510 | 1130 | 8100 |
| r97112 | 5720 | 3365 | | 920 | 725 | | 2508 | 1515 | 8065 | 22818 |
| r02089 | 8683 | 218 | | 1510 | 2493 | | 1278 | 1943 | 4670 | 20793 |
| r95116 | 6120 | | | 1495 | 143 | | 1343 | 358 | 170 | 9628 |
| r99063 | 3225 | 295 | | 100 | | | 358 | 1235 | 288 | 5500 |
| Average | 5068 | 716 | 142 | 730 | 498 | 8 | 2056 | 1282 | 1861 | 12,360 |

Breadth of vaccine induced immune responses (number of unique epitopes)

| | | | | | | | | | | |
|---|---|---|---|---|---|---|---|---|---|---|
| r00061 | 7 | | | 2 | | | 2 | 1 | 1 | 13 |
| r02103 | 9 | 2 | | | 2 | 1 | 4 | 1 | 2 | 21 |
| r02114 | 11 | 2 | 1 | 2 | 1 | | 4 | 2 | | 23 |
| r01099 | 9 | 2 | | 2 | 2 | | 3 | 1 | 3 | 22 |
| r97112 | 8 | 2 | | 3 | 1 | | 5 | 1 | 4 | 24 |
| r02089 | 13 | 1 | | 4 | 1 | | 5 | 1 | 9 | 34 |
| r95116 | 5 | | | 3 | 1 | | 2 | 1 | 1 | 13 |
| r99063 | 4 | 2 | | 1 | | | 1 | 1 | 2 | 11 |
| Average | 8 | 1 | 0 | 2 | 1 | 0 | 3 | 1 | 3 | 20 |

TABLE 2
Percentage difference in amino acid sequence between SIVmac239 and SIVsmE660

| | Protein | | | | | | | | | | | | | |
|---|---|---|---|---|---|---|---|---|---|---|---|---|---|---|
| | | | | | | | | Pol- | Pol- | Pol- | | Gag- | Gag- | Gag- |
| | Tat | Rev | Nef | Vif | Env | Vpr | Pol | Pro | RT | Int | Gag | p15 | p27 | p18 |
| % aa | 26 | 25 | 21.3 | 17 | — | 12 | 8.3 | 11.1 | 6.2 | 5.1 | 7.8 | 5.2 | 4.8 | 15 |
| difference | | | | | | | | | | | | | | |

TABLE 3
The vaccine does not offer any protection against acquisition
of SIV infection, where no difference was observed in the number
of challenges required to infect vaccines versus controls and
each group required an average of 4 challenges.

| | | | |
|---|---|---|---|
| | | | total |
| | | | number of |
| Animal ID | 800 TCID₅₀ | 4000 TCID₅₀ | challenges |

Vaccinees

| | | | |
|---|---|---|---|
| r00061 | 1 | | 1 |
| r02103 | 1 | | 1 |
| r02114 | 1 | | 1 |
| r01099 | 2 | | 2 |
| r95116 | 3 | | 3 |
| r99063 | 5 | 1 | 6 |
| r97112 | 5 | 3 | 8 |
| r02089 | 5 | 5 | 10 |
| Average | | | 4 |

Controls

| | | | |
|---|---|---|---|
| r02111 | 1 | | 1 |
| r02012 | 2 | | 2 |
| r02058 | 2 | | 2 |
| r96053 | 2 | | 2 |
| r95117 | 3 | | 3 |
| r02021 | 4 | | 4 |
| r02020 | 5 | 2 | 7 |
| r00069 | 5 | 6 | 11 |
| Average | | | 4 |

TABLE 4
Summary of the viral loads of controls and vaccines
from day 0 through day 140 post-infection.

| | log10(vRNA copy Eq/ml plasma + 1) | | | | | | | | P-values | |
|---|---|---|---|---|---|---|---|---|---|---|
| | Controls | | | | Vaccinees | | | | Two | Permu- |
| | | Mean | | Std | | Mean | | Std | sample | tation |
| Days | N | (log) | Mean | Dev | N | (log) | Mean | Dev | t-test | test\hat{ }\hat{ } |
| 0 | 8 | 1.5 | 31 | 0.0 | 8 | 1.5 | 31 | 0.0 | 1.0000 | 1.0000 |
| 7 | 8 | 3.5 | 2,999 | 1.3 | 7 | 3.0 | 1,001 | 1.3 | 0.5002 | 0.5896 |
| 9 | 8 | 4.5 | 34,358 | 1.3 | 8 | 3.5 | 2,955 | 1.7 | 0.1755 | 0.1699 |
| 11 | 7 | 5.9 | 852,170 | 1.1 | 8 | 4.0 | 10,188 | 2.0 | 0.0410\hat{ } | 0.0440\hat{ } |
| 14 | 8 | 6.3 | 1,826,271 | 1.0 | 8 | 4.1 | 13,916 | 2.1 | 0.0272\hat{ } | 0.0315\hat{ } |
| 21 | 8 | 5.8 | 567,532 | 1.2 | 8 | 2.9 | 804 | 1.7 | 0.0020\hat{ } | 0.0037\hat{ } |
| 28 | 6 | 5.3 | 215,246 | 1.6 | 8 | 2.5 | 322 | 1.5 | 0.0072\hat{ } | 0.0057\hat{ } |
| 42 | 7 | 5.0 | 98,083 | 1.6 | 8 | 2.3 | 213 | 1.4 | 0.0048\hat{ } | 0.0028\hat{ } |
| 56 | 6 | 5.1 | 119,343 | 1.3 | 7 | 2.3 | 215 | 1.4 | 0.0040\hat{ } | 0.0076\hat{ } |
| 84 | 6 | 4.5 | 30,750 | 1.0 | 6 | 2.7 | 530 | 2.0 | 0.0940 | 0.0801 |
| 98 | 1 | 5.6 | 365,001 | | 3 | 1.8 | 58 | 0.5 | | |
| 112 | 5 | 4.5 | 33,587 | 1.5 | 5 | 1.8 | 64 | 0.4 | 0.0124\hat{ } | 0.0079\hat{ } |
| 140 | 4 | 4.4 | 23,775 | 1.8 | 5 | 2.1 | 126 | 0.9 | 0.0796 | 0.0397\hat{ } |
| Peak^{a} | 8 | 6.4 | 2,463,240 | 0.9 | 8 | 4.5 | 32,325 | 1.8 | 0.0263\hat{ } | 0.0247\hat{ } |
| Average^{b} | 7 | 4.9 | 77,104 | 1.6 | 8 | 2.3 | 201 | 1.4 | 0.0059\hat{ } | 0.0090\hat{ } |

Non-missing viral concentrations lower than 30 are set to 30 prior to log-transformation.
^{a}Peak viral load was determined using the highest viral load for each animal. While the peak of viremia usually occurs at 14 days post infection, some animals have an early or delayed peak, therefore this number is slightly higher than the day 14 mean viral loads for each group.
^{b}across days 42 through 140.
\hat{ }P-value < 0.05
\hat{ }\hat{ }Permutation test is not applied when group samples are smaller than four.

TABLE 5
Summary of SGA sequence analysis and the number of transmitted viruses

| | | | | | |
|---|---|---|---|---|---|
| | Days to | | No. of SGA | No. of G-toA | No. of |
| | sampling post | | derivied | hypermutated | transmitted |
| Subject | infection | Viral load | sequences | sequences | viruses |

Titration

| | | | | | |
|---|---|---|---|---|---|
| r96087^{a} | 10 | 11,900,000 | 70 | 1 | >5 |
| rh2156^{a} | 10 | 1,460,000 | 73 | 4 | 3 |
| r92093^{b} | 9 | 264 | 18 | 8 | 1 |
| | 14 | 3,430,000 | 27 | 13 | |
| r96096^{b} | 14 | 42,000 | 19 | 5 | 1 |

Control

| | | | | | |
|---|---|---|---|---|---|
| r02111^{c} | 14 | 667,000 | 20 | 1 | 1 |
| r02012^{c} | 15 | 146,000,000 | 22 | 1 | 4 |
| r02058^{c} | 15 | 7,340,000 | 17 | 0 | 3 |
| r96053^{c} | 7 | 398 | 3 | 0 | 4 |
| | 14 | 6,310,000 | 17 | 4 | |
| r95117^{c} | 12 | 41,200 | 21 | 0 | 1 |
| r02021^{c} | 9 | 218,000 | 21 | 0 | 3 |
| r02020^{a} | 14 | 87,200 | 19 | 0 | 1 |
| r00069^{a} | 14 | 483,000 | 20 | 0 | 1 |

Vaccine

| | | | | | |
|---|---|---|---|---|---|
| r02103^{c} | 9 | 283 | 10 | 8 | 1 |
| r02114^{c} | 14 | 71,200 | 23 | 13 | 1 |
| r01099^{c} | 7 | 1,620 | 17 | 3 | 4 |
| | 15 | 201,000 | 28 | 4 | |
| r95116^{c} | 12 | 47,500 | 23 | 0 | 2 |
| r99063^{a} | 15 | 8,520,000 | 18 | 1 | 1 |
| r97112^{a} | 7 | 5,540 | 14 | 1 | 1 |
| r02089^{a} | 14 | 1,390 | 12 | 3 | 2 |

^{a}Animals infected IR with 4,000 TCID50 (6 × 10⁷ RNA copies)
^{b}Animals infected IR with 400 TCID50 (6 × 10⁶ RNA copies)
^{c}Animals infected IR with 800 TCID50 (1.2 × 10⁷ RNA copies)

TABLE 6
Frequency and breadth of anamnestic cellular immune
responses in PBMC and PBMC depleted of CD8+ cells.

| | | | | | | | | | | |
|---|---|---|---|---|---|---|---|---|---|---|
| Animal | | | | | | | | | | |
| ID | Gag | Nef | Tat | Rev | Vpr | Vpx | Vif | Env | Pol | Total |

Frequency of anamnestic whole PBMC
responses (SFC/10⁶ PBMC).

| | | | | | | | | | | |
|---|---|---|---|---|---|---|---|---|---|---|
| r00061 | 718 | 120 | | 116 | | | 58 | | 128 | 1,140 |
| r02103 | 2,738 | 600 | | | | | 690 | 240 | 912 | 5,180 |
| r02114 | 1,455 | 170 | 205 | | | | 525 | 320 | 100 | 2,775 |
| r01099 | 5,457 | 4,286 | | | | | 475 | | | 10,218 |
| r97112 | 11,259 | 360 | | | | | | | 1,540 | 13,159 |
| r02089 | 4,890 | 255 | | 205 | 610 | | | 155 | 670 | 6,785 |
| r95116 | 8,532 | 0 | | | | | 603 | 83 | 215 | 9,433 |
| r99063 | 16,178 | | | | | | 318 | | 205 | 16,701 |
| Average | 6,403 | 724 | 26 | 40 | 76 | 0 | 334 | 100 | 471 | 8,174 |

Breadth of anamnestic PBMC responses
(number of unique epitopes)

| | | | | | | | | | | |
|---|---|---|---|---|---|---|---|---|---|---|
| r00061 | 2 | 1 | | 2 | | | 2 | | 1 | 8 |
| r02103 | 2 | 1 | | | | | 1 | 1 | 4 | 9 |
| r02114 | 7 | 1 | 1 | | | | 3 | 1 | 1 | 14 |
| r01099 | 5 | 2 | | | | | 1 | | | 8 |
| r97112 | 4 | 1 | | | | | | | 5 | 10 |
| r02089 | 10 | 1 | | 1 | 1 | | | 1 | 2 | 16 |
| r95116 | 4 | | | | | | 2 | 1 | 2 | 9 |
| r99063 | 3 | | | | | | 1 | | 1 | 5 |
| Average | 5 | 1 | 0 | 0 | 0 | 0 | 1 | 1 | 2 | 10 |

Frequency of anamnestic CD8-depleted
PBMC responses (SFC/106 PBMC).

| | | | | | | | | | | |
|---|---|---|---|---|---|---|---|---|---|---|
| r00061 | | | | | | | | | | 0 |
| r02103 | 464 | 78 | | | | | | 78 | 153 | 773 |
| r02114 | 1,960 | | | | | | 70 | | 80 | 2,110 |
| r01099 | 890 | | | 125 | | | | | | 1,015 |
| r97112 | 1,590 | | | 250 | | | | | 120 | 1,960 |
| r02089 | 7,044 | | 137 | 1,236 | | | 751 | | 1,342 | 10,510 |
| r95116 | 405 | | | 110 | | | | | 90 | 605 |
| r99063 | 234 | | | | | | | | | 234 |
| Average | 1,573 | 10 | 17 | 215 | 0 | 0 | 103 | 10 | 223 | 2,151 |

Breadth of anamnestic CP8-depleted PBMC
responses (number of unique epitopes)

| | | | | | | | | | | |
|---|---|---|---|---|---|---|---|---|---|---|
| r00061 | | | | | | | | | | 0 |
| r02103 | 3 | 1 | | | | | | 1 | 1 | 6 |
| r02114 | 5 | | | | | | 1 | | 1 | 7 |
| r01099 | 6 | | | 1 | | | | | | 7 |
| r97112 | 4 | | | 2 | | | | | 1 | 7 |
| r02089 | 12 | | 1 | 3 | | | 3 | | 6 | 25 |
| r95116 | 3 | | | 1 | | | | | 1 | 5 |
| r99063 | 2 | | | | | | | | | 2 |
| Average | 4 | 0 | 0 | 1 | 0 | 0 | 1 | 0 | 1 | 7 |

TABLE 7
Mamu = A*02-bound SIVmac239-derived binding
minimal optimal peptides have several changes
when compared with the complementary
peptide in SIVsmE660.

| | | SIVmac239 | SIVsmE660 |
|---|---|---|---|
| Protein | position | sequence | sequence |
| Env | 296-304 | RTIISLNKY | --------- |
| | 317-325 | KTVLPVTIM | --------- |
| | 359-367 | QTIVKHPRY | E-L------ |
| | 519-528 | GTSRNKRGVF | -A-------- |
| | 760-768 | SSWPWQIEY | R-------- |
| | 788-795 | RTLLSRVY | -DW-L-t- |
| Gag | 71-79 | GSENLKSLY | --------- |
| Nef | 20-28 | LLRARGETY | --Q------ |
| | 110-119 | TMSYKLAIDM | A-T------- |
| | 159-167 | YTSGPGIRY | ------T-- |
| | 169-177 | KTFGWLWKL | MYY------ |
| | 221-229 | YTYEAYVRY | -S-K-FIK- |
| | 248-256 | LTARGLLNM | ------IK- |
| Vif | 89-97 | ITWYSKNFW | -----R--- |
| | 97-104 | WTDVTPNY | ------D- |
| | 104-113 | YADILLHSTY | ---T------ |
| Vpr | 63-71 | RILQRALFM | --------I |
| Pol | 324-332 | FSIPLDEEF | --------- |
| | 518-526 | LSQEQEGCY | --------- |

Dashes represent exact matches. Of the 21 immunogenic SIVmac239 peptides known to bind to Mamu-A*02, only 5 are completely conserved in SIVsm660.

TABLE 8
The Frequency and Breadth of vaccine-induced cellular immune
responses was measured in PBMC and in PBMC depleted of CD8+
cells at or near to 33 weeks after the Ad5 boost, a month
prior to the initiation of intrarectal challenges.

| | | | | | | | | | | |
|---|---|---|---|---|---|---|---|---|---|---|
| Animal | | | | | | | | | | |
| ID | Gag | Nef | Tat | Rev | Vpr | Vpx | Vif | Env | Pol | Total |

Frequency of vaccine-induced immune responses
in PBMC prior to challenge (SFC/10⁶ PBMC).

| | | | | | | | | | | |
|---|---|---|---|---|---|---|---|---|---|---|
| r00061 | 561 | 67 | 0 | 0 | 0 | 0 | 138 | 0 | 120 | 886 |
| r02103 | 139 | 67 | 0 | 0 | 0 | 0 | 332 | 0 | 313 | 851 |
| r02114 | 487 | 213 | 0 | 0 | 0 | 0 | 733 | 0 | 386 | 1819 |
| r01099 | 63 | 358 | 0 | 0 | 0 | 0 | 113 | 0 | 140 | 674 |
| r97112 | 610 | 685 | 0 | 0 | 0 | 0 | 495 | 170 | 1785 | 3745 |
| r02089 | 1742 | 170 | 0 | 0 | 232 | 0 | 0 | 0 | 820 | 2964 |
| r95116 | 1707 | 102 | 0 | 0 | 138 | 0 | 122 | 0 | 129 | 2198 |
| r99063 | 420 | 70 | 0 | 0 | 0 | 0 | 95 | 0 | 190 | 775 |
| Average | 716 | 217 | 0 | 0 | 46 | 0 | 254 | 21 | 485 | 1739 |

Breadth of vaccine-induced immune responses
in PBMC prior to challenge

| | | | | | | | | | | |
|---|---|---|---|---|---|---|---|---|---|---|
| r00061 | 3 | 1 | 0 | 0 | 0 | 0 | 2 | 0 | 1 | 7 |
| r02103 | 3 | 1 | 0 | 0 | 0 | 0 | 1 | 0 | 4 | 9 |
| r02114 | 2 | 1 | 0 | 0 | 0 | 0 | 3 | 0 | 4 | 10 |
| r01099 | 1 | 2 | 0 | 0 | 0 | 0 | 1 | 0 | 1 | 5 |
| r97112 | 1 | 1 | 0 | 0 | 0 | 0 | 1 | 1 | 5 | 9 |
| r02089 | 3 | 1 | 0 | 0 | 1 | 0 | 0 | 0 | 2 | 7 |
| r95116 | 3 | 1 | 0 | 0 | 1 | 0 | 1 | 0 | 2 | 8 |
| r99063 | 2 | 1 | 0 | 0 | 0 | 0 | 1 | 0 | 1 | 5 |
| Average | 2 | 1 | 0 | 0 | 0 | 0 | 1 | 0 | 3 | 8 |

Frequency of vaccine-induced immune responses in CD8
depleted PBMC prior to challenge (SFC/10⁶ PBMC).

| | | | | | | | | | | |
|---|---|---|---|---|---|---|---|---|---|---|
| r00061 | 60 | 0 | 0 | 0 | 0 | 0 | 0 | 0 | 0 | 60 |
| r02103 | 55 | 0 | 0 | 0 | 0 | 0 | 0 | 0 | 0 | 55 |
| r02114 | 0 | 0 | 0 | 0 | 0 | 0 | 0 | 0 | 0 | 0 |
| r01099 | 0 | 0 | 0 | 0 | 0 | 0 | 0 | 0 | 0 | 0 |
| r97112 | 0 | 0 | 0 | 123 | 0 | 0 | 0 | 0 | 0 | 123 |
| r02089 | 1474 | 0 | 143 | 296 | 0 | 0 | 53 | 0 | 257 | 2223 |
| r95116 | 57 | 0 | 0 | 50 | 0 | 0 | 0 | 0 | 0 | 107 |
| r99063 | 0 | 0 | 0 | 0 | 0 | 0 | 0 | 0 | 0 | 0 |
| Average | 206 | 0 | 18 | 59 | 0 | 0 | 7 | 0 | 32 | 321 |

Breadth of vaccine-induced immune responses
in CD8 depleted PBMC prior to challenge

| | | | | | | | | | | |
|---|---|---|---|---|---|---|---|---|---|---|
| r00061 | 1 | 0 | 0 | 0 | 0 | 0 | 0 | 0 | 0 | 1 |
| r02103 | 1 | 0 | 0 | 0 | 0 | 0 | 0 | 0 | 0 | 1 |
| r02114 | 0 | 0 | 0 | 0 | 0 | 0 | 0 | 0 | 0 | 0 |
| r01099 | 0 | 0 | 0 | 0 | 0 | 0 | 0 | 0 | 0 | 0 |
| r97112 | 0 | 0 | 0 | 1 | 0 | 0 | 0 | 0 | 0 | 1 |
| r02089 | 8 | 0 | 1 | 2 | 0 | 0 | 1 | 0 | 4 | 16 |
| r95116 | 1 | 0 | 0 | 1 | 0 | 0 | 0 | 0 | 0 | 2 |
| r99063 | 0 | 0 | 0 | 0 | 0 | 0 | 0 | 0 | 0 | 0 |
| Average | 1 | 0 | 0 | 1 | 0 | 0 | 0 | 0 | 1 | 3 |

TABLE 9
Mean percentages of maximum suppression of SIVmac239
replication of individual vaccinated and control
animals at 3 effector:target ratios.

| | Maximum suppression of SIVmac239 | | | | | | | | |
|---|---|---|---|---|---|---|---|---|---|
| | replication at E:T ratio^{a}: | | | | | | | | |
| | 1:1 | | | 0.5:1 | | | 0.1:1 | | |
| Group and | % Suppression | | | % Suppression | | | % Suppression | | |
| animal | Mean | SD | n | Mean | SD | n | Mean | SD | n |
| Vaccinated | | | | | | | | | |
| group | | | | | | | | | |
| r00061 | 68.00 | 9.17 | 3 | 44.67 | 12.66 | 3 | 15.67 | 6.43 | 3 |
| r01099 | 79.00 | 8.89 | 3 | 69.00 | 10.44 | 3 | 42.67 | 21.08 | 3 |
| r02089 | 48.33 | 30.29 | 3 | 51.00 | 15.72 | 3 | −8.33 | 16.07 | 3 |
| r02103 | 59.67 | 22.37 | 3 | 43.33 | 20.82 | 3 | 23.33 | 6.03 | 3 |
| r02114 | 48.00 | 19.31 | 3 | 21.33 | 14.22 | 3 | 17.67 | 17.95 | 3 |
| r97112 | 99.00 | 1.00 | 3 | 94.33 | 1.53 | 3 | 74.67 | 5.51 | 3 |
| r99063 | 64.00 | 2.83 | 2 | 44.67 | 18.82 | 3 | 12.67 | 13.61 | 3 |
| r95116 | 93.33 | 2.08 | 3 | 82.67 | 8.08 | 3 | 52.67 | 5.86 | 3 |
| Control | | | | | | | | | |
| group | | | | | | | | | |
| r00069 | 5.00 | 0.00 | 1 | 8.00 | 0.00 | 1 | 11.00 | 0.00 | 1 |
| r02012 | 19.00 | 0.00 | 1 | 37.00 | 0.00 | 1 | −10.00 | 0.00 | 1 |
| r02020 | 31.00 | 0.00 | 1 | 8.00 | 0.00 | 1 | 2.00 | 0.00 | 1 |
| r02021 | 20.00 | 0.00 | 1 | −2.00 | 0.00 | 1 | 9.00 | 0.00 | 1 |
| r02058 | 36.00 | 0.00 | 1 | 18.00 | 0.00 | 1 | −12.00 | 0.00 | 1 |
| r02111 | 19.00 | 0.00 | 1 | 17.00 | 0.00 | 1 | 7.00 | 0.00 | 1 |
| r95117 | 18.33 | 3.21 | 3 | −1.00 | 32.91 | 3 | 11.00 | 40.34 | 3 |
| r96053 | 33.33 | 35.85 | 3 | 15.00 | 18.52 | 3 | 5.00 | 13.23 | 3 |

^{a}n, Number of independent measurements.

TABLE 10
Mean percentages of maximum suppression of SIVsmE660
replication of individual vaccinated and control
animals at 3 effector:target ratios.

| | Maximum suppression of SIVsmE660 | | | | | | | | |
|---|---|---|---|---|---|---|---|---|---|
| | replication at E:T ratio^{a}: | | | | | | | | |
| | 1:1 | | | 0.5:1 | | | 0.1:1 | | |
| Group and | % Suppression | | | % Suppression | | | % Suppression | | |
| animal | Mean | SD | n | Mean | SD | n | Mean | SD | n |
| Vaccinated | | | | | | | | | |
| group | | | | | | | | | |
| r00061 | 62.50 | 10.61 | 2 | 43.00 | 12.73 | 2 | 15.00 | 2.83 | 2 |
| r01099 | 63.50 | 16.26 | 2 | 56.50 | 7.78 | 2 | 14.50 | 17.68 | 2 |
| r02089 | 74.00 | 15.56 | 2 | 63.50 | 10.61 | 2 | 13.50 | 13.44 | 2 |
| r02103 | 58.00 | 36.77 | 2 | 57.00 | 26.87 | 2 | 15.00 | 41.01 | 2 |
| r02114 | 31.45 | 16.14 | 2 | 4.49 | 28.67 | 2 | −7.45 | 26.70 | 2 |
| r97112 | 93.56 | 2.33 | 2 | 92.10 | 1.44 | 2 | 65.44 | 8.50 | 2 |
| r99063 | 36.19 | 1.15 | 2 | 18.87 | 2.86 | 2 | 3.36 | 7.08 | 2 |
| r95116 | 62.47 | 2.20 | 2 | 43.92 | 1.90 | 2 | 27.54 | 4.36 | 2 |
| Control | | | | | | | | | |
| group | | | | | | | | | |
| r00069 | 22.12 | 35.42 | 2 | −0.61 | 24.74 | 2 | 12.98 | 17.42 | 2 |
| r02012 | 43.47 | 13.13 | 2 | 30.17 | 13.12 | 2 | 10.20 | 19.45 | 2 |
| r02020 | 65.13 | 8.28 | 2 | 35.08 | 19.81 | 2 | 8.66 | 15.37 | 2 |
| r02021 | 34.00 | 9.96 | 2 | 16.75 | 1.70 | 2 | 8.03 | 12.64 | 2 |
| r02058 | 19.15 | 8.97 | 2 | 7.23 | 6.03 | 2 | 1.69 | 2.03 | 2 |
| r02111 | 48.13 | 9.72 | 2 | 29.13 | 19.89 | 2 | −6.63 | 20.50 | 2 |
| r95117 | 23.00 | 5.66 | 2 | −16.00 | 43.84 | 2 | −2.00 | 9.90 | 2 |
| r96053 | 15.44 | 2.28 | 2 | 13.69 | 21.51 | 2 | −7.38 | 18.58 | 2 |

^{a}n, Number of independent measurements.

TABLE 11
Summary of viral loads and absolute CD4+ T-cell counts
for vaccinated animals in both acute and chronic phases.

| | Plasma viral loads | | | | Absolute no. of CD4⁺ T | |
|---|---|---|---|---|---|---|
| | (vRNA copies/ml)^{a} | | | | cells/μl of blood^{b} | |
| | | | Set | | Acute | Chronic |
| Animal | Peak viremia | | point viremia | | phase | phase |
| r00061 | 220 | (2.3) | 0 | (0) | 1,122 | 2,868 |
| r01099 | 446,000 | (5.6) | 2 | (0.3) | 683 | 576 |
| r02089 | 1,390 | (3.1) | 0 | (0) | 867 | 1,389 |
| r02103 | 302 | (2.5) | 0 | (0) | 1,026 | 1,728 |
| r02114 | 71,200 | (4.9) | 57 | (1.8) | 886 | 1,177 |
| r97112 | 8,360 | (3.9) | 20 | (1.3) | 848 | 1,309 |
| r99063 | 24,100,000 | (7.4) | 585,907 | (5.8) | 926 | 205 |
| r95116 | 2,000,000 | (6.3) | 2,913 | (3.5) | 633 | 495 |

^{a}Values are given as absolute values, with the log-transformed values in parentheses. Peak viremia was defined as the highest viral load measurement within the first 4 weeks of infection. Set point viremia was defined as the average of log-transformed viral loads at weeks 6 to 24 postinfection.
^{b}Acute-phase values were obtained at weeks 2 to 3 postinfection. Chronic-phase values were obtained at week 24 postinfection.

TABLE 12^{a}
Pre-challenge^{b} vaccine-induced cellular responses (frequency and breadth^{c}) detected in PBMC of vaccinees.

| Parameter (frequency or breadth of | Prechallenge vaccine-induced cellular response | | | | | | | | | |
|---|---|---|---|---|---|---|---|---|---|---|
| response) and animal | Gag | Nef | Tat | Rev | Vpr | Vpx | Vif | Env | Pol | Total |
| Frequency of vaccine-induced immune | | | | | | | | | | |
| responses (SFC/10⁶ PBMC) | | | | | | | | | | |
| r02103 | 3,258 | 785 | 0 | 0 | 255 | 60 | 2,675 | 163 | 328 | 7,523 |
| r02114 | 7,183 | 385 | 1,133 | 305 | 63 | 0 | 6,565 | 3,425 | 0 | 19,058 |
| r01099 | 2,745 | 680 | 0 | 1,000 | 303 | 0 | 733 | 1,510 | 1,130 | 8,100 |
| r97112 | 5,720 | 3,365 | 0 | 920 | 725 | 0 | 2,508 | 1,515 | 8,065 | 22,818 |
| r02089 | 8,683 | 218 | 0 | 1,510 | 2,493 | 0 | 1,278 | 1,943 | 4,670 | 20,793 |
| r95116 | 6,120 | 0 | 0 | 1,495 | 143 | 0 | 1,343 | 358 | 170 | 9,628 |
| r99063 | 3,225 | 295 | 0 | 100 | 0 | 0 | 358 | 1,235 | 288 | 5,500 |
| Avg | 5,276 | 818 | 162 | 761 | 569 | 9 | 2,209 | 1,450 | 2,093 | 13,346 |
| Median | 5,720 | 385 | 0 | 920 | 255 | 0 | 1,343 | 1,510 | 328 | 9,628 |
| Breadth of vaccine-induced immune | | | | | | | | | | |
| responses (no. of unique epitopes) | | | | | | | | | | |
| r02103 | 9 | 2 | 0 | 0 | 2 | 1 | 4 | 1 | 2 | 21 |
| r02114 | 11 | 2 | 1 | 2 | 1 | 0 | 4 | 2 | 0 | 23 |
| r01099 | 9 | 2 | 0 | 2 | 2 | 0 | 3 | 1 | 3 | 22 |
| r97112 | 8 | 2 | 0 | 3 | 1 | 0 | 5 | 1 | 4 | 24 |
| r02089 | 13 | 1 | 0 | 4 | 1 | 0 | 5 | 1 | 9 | 34 |
| r95116 | 5 | 0 | 0 | 3 | 1 | 0 | 2 | 1 | 1 | 13 |
| r99063 | 4 | 2 | 0 | 1 | 0 | 0 | 1 | 1 | 2 | 11 |
| Avg | 8 | 2 | 0 | 2 | 1 | 0 | 3 | 1 | 3 | 21 |
| Median | 9 | 2 | 0 | 2 | 1 | 0 | 4 | 1 | 2 | 22 |

^{a}Adapted from Wilson et al. (59). Breadth was defined as the number of single 15-mers for which positive ELISPOT assay responses were observed. An ELISPOT assay was considered positive if it was ≧50 SFC/10⁶ PBMC and ≧2 standard deviations over the background. Prechallenge, measured on day 14 after vaccination.

TABLE 13^{a}
Post-challenge^{b} anamnestic cellular responses (frequency and breadth^{c}) detected in PBMC of vaccinees.

| Parameter (frequency or breadth of | Postchallenge anamnestic cellular response | | | | | | | | | |
|---|---|---|---|---|---|---|---|---|---|---|
| response) and animal | Gag | Nef | Tat | Rev | Vpr | Vpx | Vif | Env | Pol | Total |
| Frequency of anamnestic immune responses | | | | | | | | | | |
| in PBMC (SFC/10⁶ PBMC) | | | | | | | | | | |
| r02103 | 2,738 | 600 | 0 | 0 | 0 | 0 | 690 | 240 | 912 | 5,180 |
| r02114 | 1,455 | 170 | 205 | 0 | 0 | 0 | 525 | 320 | 100 | 2,775 |
| r01099 | 5,457 | 4,286 | 0 | 0 | 0 | 0 | 475 | 0 | 0 | 10,218 |
| r97112 | 11,259 | 360 | 0 | 0 | 0 | 0 | 0 | 0 | 1,540 | 13,159 |
| r02089 | 4,890 | 255 | 0 | 205 | 610 | 0 | 0 | 155 | 670 | 6,785 |
| r95116 | 8,532 | 0 | 0 | 0 | 0 | 0 | 603 | 83 | 215 | 9,433 |
| r99063 | 16,178 | 0 | 0 | 0 | 0 | 0 | 318 | 0 | 205 | 16,701 |
| Avg | 7,216 | 810 | 29 | 29 | 87 | 0 | 373 | 114 | 520 | 9,179 |
| Median | 5,457 | 255 | 0 | 0 | 0 | 0 | 475 | 83 | 215 | 9,433 |
| Frequency of anamnestic immune responses | | | | | | | | | | |
| in CD8⁻ PBMC (SFC/10⁶ PBMC) | | | | | | | | | | |
| r02103 | 464 | 78 | 0 | 0 | 0 | 0 | 0 | 78 | 153 | 773 |
| r02114 | 1,960 | 0 | 0 | 0 | 0 | 0 | 70 | 0 | 80 | 2,110 |
| r01099 | 890 | 0 | 0 | 125 | 0 | 0 | 0 | 0 | 0 | 1,015 |
| r97112 | 1,590 | 0 | 0 | 250 | 0 | 0 | 0 | 0 | 120 | 1,960 |
| r02089 | 7,044 | 0 | 137 | 1,236 | 0 | 0 | 751 | 0 | 1,342 | 10,510 |
| r95116 | 405 | 0 | 0 | 110 | 0 | 0 | 0 | 0 | 90 | 605 |
| r99063 | 234 | 0 | 0 | 0 | 0 | 0 | 0 | 0 | 0 | 234 |
| Avg | 1,798 | 11 | 20 | 246 | 0 | 0 | 117 | 11 | 255 | 2,458 |
| Median | 890 | 0 | 0 | 110 | 0 | 0 | 0 | 0 | 90 | 1,015 |
| Breadth of anamnestic immune responses | | | | | | | | | | |
| in PBMC (no. of unique epitopes) | | | | | | | | | | |
| r02103 | 2 | 1 | 0 | 0 | 0 | 0 | 1 | 1 | 4 | 9 |
| r02114 | 7 | 1 | 1 | 0 | 0 | 0 | 3 | 1 | 1 | 14 |
| r01099 | 5 | 2 | 0 | 0 | 0 | 0 | 1 | 0 | 0 | 8 |
| r97112 | 4 | 1 | 0 | 0 | 0 | 0 | 0 | 0 | 5 | 10 |
| r02089 | 10 | 1 | 0 | 1 | 1 | 0 | 0 | 1 | 2 | 16 |
| r95116 | 4 | 0 | 0 | 0 | 0 | 0 | 2 | 1 | 2 | 9 |
| r99063 | 3 | 0 | 0 | 0 | 0 | 0 | 1 | 0 | 1 | 5 |
| Avg | 5 | 1 | 0 | 0 | 0 | 0 | 1 | 1 | 2 | 10 |
| Median | 4 | 1 | 0 | 0 | 0 | 0 | 1 | 1 | 2 | 9 |

^{a}Adapted from Wilson et al. (59). Breadth was defined as the number of single 15-mers for which positive ELISPOT assay responses were observed. An ELISPOT assay was considered positive if it was ≧50 SFC/10⁶ PBMC and ≧2 standard deviations over the background. Postchallenge, measured on day 14 or 15 after infection.

TABLE 14
Correlations between the magnitude of vaccine-induced T-cell responses directed
against combinations of viral proteins and markers of disease progression.

| | | | | Accessory + | Regu- | Acces- | | | | | | |
|---|---|---|---|---|---|---|---|---|---|---|---|---|
| | | Structural | Gag + | Regulatory | latory | sory | Nef + | Nef + | Nef + | Vif + | Vif + | Vpr + |
| | | proteins | Pol | proteins | proteins | proteins | Vif | Vpr | Vpx | Vpr | Vpx | Vpx |
| | | Pre-challenge frequency of responses | | | | | | | | | | |
| Peak | r | −0.17857 | −0.39286 | −0.60714 | 0.07143 | −0.64286 | −0.67857 | −0.78571 | −0.46429 | −0.64286 | −0.57143 | −0.78571 |
| viremia | p- | 0.7017 | 0.3833 | 0.1482 | 0.879 | 0.1194 | 0.0938 | 0.0362 | 0.2939 | 0.1194 | 0.1802 | 0.0362 |
| | value | | | | | | | | | | | |
| Setpoint | r | −0.05406 | −0.25226 | −0.30632 | −0.03604 | −0.41443 | −0.41443 | −0.73877 | −0.36037 | −0.39641 | −0.23424 | −0.82886 |
| viremia | p- | 0.9084 | 0.5852 | 0.504 | 0.9389 | 0.3553 | 0.3553 | 0.0579 | 0.4271 | 0.3786 | 0.6132 | 0.0212 |
| | value | | | | | | | | | | | |
| CD4+ | r | −0.42857 | −0.33333 | 0.04762 | −0.52381 | 0.14286 | 0.2381 | 0.04762 | 0.2381 | 0.14286 | 0.14286 | −0.04762 |
| T-cell | p- | 0.1765 | 0.2931 | 0.8806 | 0.0985 | 0.6523 | 0.4527 | 0.8806 | 0.4527 | 0.6523 | 0.6523 | 0.8806 |
| counts - | value | | | | | | | | | | | |
| Acute | | | | | | | | | | | | |
| phase | | | | | | | | | | | | |
| CD4+ | r | 0.17857 | 0.39286 | 0.60714 | −0.07143 | 0.64286 | 0.67857 | 0.78571 | 0.46429 | 0.64286 | 0.57143 | 0.78571 |
| T-cell | p- | 0.7017 | 0.3833 | 0.1482 | 0.879 | 0.1194 | 0.0938 | 0.0362 | 0.2939 | 0.1194 | 0.1802 | 0.0362 |
| counts - | value | | | | | | | | | | | |
| Chronic | | | | | | | | | | | | |
| phase | | | | | | | | | | | | |
| | | Post-challenge frequency of responses | | | | | | | | | | |
| Peak | r | 0.5 | 0.5 | −0.5 | −0.31623 | −0.5 | −0.07143 | −0.59462 | −0.59462 | −0.53571 | −0.01802 | −0.40825 |
| viremia | p- | 0.2532 | 0.2532 | 0.2532 | 0.4896 | 0.2532 | 0.879 | 0.1591 | 0.1591 | 0.2152 | 0.9694 | 0.3632 |
| | value | | | | | | | | | | | |
| Setpoint | r | 0.50452 | 0.50452 | −0.77481 | −0.23932 | −0.77481 | −0.19821 | −0.87273 | −0.78182 | −0.55858 | 0.02727 | −0.51493 |
| viremia | p- | 0.2482 | 0.2482 | 0.0408 | 0.6053 | 0.0408 | 0.6701 | 0.0104 | 0.0378 | 0.1925 | 0.9537 | 0.237 |
| | value | | | | | | | | | | | |
| CD4+ | r | −0.21429 | −0.21429 | −0.03571 | 0.15811 | −0.03571 | −0.07143 | −0.05406 | 0.01802 | 0.25 | 0.16217 | 0 |
| T-cell | p- | 0.6445 | 0.6445 | 0.9394 | 0.7349 | 0.9394 | 0.879 | 0.9084 | 0.9694 | 0.5887 | 0.7283 | 1 |
| counts - | value | | | | | | | | | | | |
| Acute | | | | | | | | | | | | |
| phase | | | | | | | | | | | | |
| CD4+ | r | −0.5 | −0.5 | 0.5 | 0.31623 | 0.5 | 0.07143 | 0.59462 | 0.59462 | 0.53571 | 0.01802 | 0.40825 |
| T-cell | p- | 0.2532 | 0.2532 | 0.2532 | 0.4896 | 0.2532 | 0.879 | 0.1591 | 0.1591 | 0.2152 | 0.9694 | 0.3632 |
| counts - | value | | | | | | | | | | | |
| Chronic | | | | | | | | | | | | |
| phase | | | | | | | | | | | | |

Correlation coefficients and p-values were determined by the Spearman's rank correlation test. p-values ≦ 0.05 are highlighted in yellow.

TABLE 15
Correlations between the breadth of vaccine-induced T-cell responses directed
against combinations of viral proteins and markers of disease progression.

| | | | | Accessory + | Regu- | Acces- | | | | | | |
|---|---|---|---|---|---|---|---|---|---|---|---|---|
| | | Structural | Gag + | Regulatory | latory | sory | Nef + | Nef + | Nef + | Vif + | Vif + | Vpr + |
| | | proteins | Pol | proteins | proteins | proteins | Vif | Vpr | Vpx | Vpr | Vpx | Vpx |
| | | Pre-challenge breadth of responses | | | | | | | | | | |
| Peak | r | −0.58007 | −0.62409 | −0.70921 | −0.11119 | −0.87947 | −0.77831 | −0.47725 | −0.37435 | −0.95431 | −0.96362 | −0.61079 |
| viremia | p- | 0.1722 | 0.1342 | 0.0743 | 0.8124 | 0.0091 | 0.0393 | 0.2788 | 0.4081 | 0.0008 | 0.0005 | 0.1451 |
| | value | | | | | | | | | | | |
| Setpoint | r | −0.66085 | −0.76865 | −0.60553 | −0.0748 | −0.75525 | −0.56097 | −0.55565 | −0.27833 | −0.86854 | −0.82275 | −0.74554 |
| viremia | p- | 0.1061 | 0.0435 | 0.1496 | 0.8734 | 0.0496 | 0.1901 | 0.1953 | 0.5456 | 0.0112 | 0.023 | 0.0544 |
| | value | | | | | | | | | | | |
| CD4+ | r | −0.09356 | −0.14684 | −0.07274 | −0.51887 | 0.35553 | 0.2965 | 0.31204 | 0.7093 | 0.16841 | 0.22237 | 0.0197 |
| T-cell | p- | 0.8419 | 0.7534 | 0.8768 | 0.2328 | 0.4338 | 0.5185 | 0.4957 | 0.0743 | 0.7181 | 0.6317 | 0.9666 |
| counts - | value | | | | | | | | | | | |
| Acute | | | | | | | | | | | | |
| phase | | | | | | | | | | | | |
| CD4+ | r | 0.58007 | 0.62409 | 0.70921 | 0.11119 | 0.87947 | 0.77831 | 0.47725 | 0.37435 | 0.95431 | 0.96362 | 0.61079 |
| T-cell | p- | 0.1722 | 0.1342 | 0.0743 | 0.8124 | 0.0091 | 0.0393 | 0.2788 | 0.4081 | 0.0008 | 0.0005 | 0.1451 |
| counts - | value | | | | | | | | | | | |
| Chronic | | | | | | | | | | | | |
| phase | | | | | | | | | | | | |
| | | Post-challenge breadth of responses | | | | | | | | | | |
| Peak | r | −0.65465 | −0.66669 | −0.22027 | −0.31623 | −0.11227 | 0.05614 | −0.56695 | −0.49801 | 0.29554 | 0.43038 | −0.40825 |
| viremia | p- | 0.1106 | 0.1019 | 0.6351 | 0.4896 | 0.8106 | 0.9049 | 0.1844 | 0.2554 | 0.5199 | 0.3351 | 0.3632 |
| | value | | | | | | | | | | | |
| Setpoint | r | −0.46791 | −0.48182 | −0.35191 | −0.23932 | −0.24546 | −0.00944 | −0.81044 | −0.70353 | 0.33798 | 0.50038 | −0.51493 |
| viremia | p- | 0.2897 | 0.2736 | 0.4389 | 0.6053 | 0.5957 | 0.984 | 0.0271 | 0.0778 | 0.4584 | 0.2528 | 0.237 |
| | value | | | | | | | | | | | |
| CD4+ | r | −0.07274 | −0.10811 | −0.09178 | 0.15811 | −0.13098 | −0.11227 | −0.09449 | −0.07968 | −0.05911 | −0.01871 | 0 |
| T-cell | p- | 0.8768 | 0.8175 | 0.8448 | 0.7349 | 0.7795 | 0.8106 | 0.8403 | 0.8652 | 0.8998 | 0.9682 | 1 |
| counts - | value | | | | | | | | | | | |
| Acute | | | | | | | | | | | | |
| phase | | | | | | | | | | | | |
| CD4+ | r | 0.65465 | 0.66669 | 0.22027 | 0.31623 | 0.11227 | −0.05614 | 0.56695 | 0.49801 | −0.29554 | −0.43038 | 0.40825 |
| T-cell | p- | 0.1106 | 0.1019 | 0.6351 | 0.4896 | 0.8106 | 0.9049 | 0.1844 | 0.2554 | 0.5199 | 0.3351 | 0.3632 |
| counts - | value | | | | | | | | | | | |
| Chronic | | | | | | | | | | | | |
| phase | | | | | | | | | | | | |

Correlation coefficients and p-values were determined by the Spearman's rank correlation test. p-values ≦ 0.05 are highlighted in yellow.

We claim:

1. A pharmaceutical composition comprising a mixture of HIV DNA polynucleotides and a pharmaceutical carrier, the mixture comprising:
(a) a first polynucleotide encoding a single HIV polypeptide consisting of HIV Gag polypeptide; and
(b) a second polynucleotide encoding a single HIV polypeptide consisting of HIV Vif polypeptide;
wherein the composition comprises an effective amount of the mixture of polynucleotides for inducing a protective or therapeutic immune response against HIV infection.

2. The composition of claim 1, the mixture further comprising:
(c) a third polynucleotide encoding a single HIV polypeptide consisting of HIV Nef polypeptide.

3. The composition of claim 1, the mixture further comprising:
(d) a fourth polynucleotide encoding a single HIV polypeptide selected from a group consisting of HIV Pol polypeptide, HIV Tat polypeptide, HIV Rev polypeptide, and HIV Vpr polypeptide.

4. The composition of claim 1, wherein each polynucleotide encodes a different amino acid sequence.

5. The composition of claim 2, wherein each polynucleotide encodes a different amino acid sequence.

6. The composition of claim 1, wherein each polynucleotide is a different polynucleotide.

7. The composition of claim 1, wherein the polynucleotides are present in a viral vector.

8. The composition of claim 7, wherein the viral vector is an adenovirus vector.

9. A pharmaceutical composition comprising a mixture of HIV DNA polynucleotides and a pharmaceutical carrier, the mixture comprising:
(a) a first polynucleotide encoding a single HIV polypeptide consisting of HIV Gag polypeptide or an immunogenic fragment thereof; and
(b) a second polynucleotide encoding a single HIV polypeptide consisting of HIV Vif polypeptide or an immunogenic fragment thereof;
wherein the composition comprises an effective amount of the mixture of polynucleotides for inducing a protective or therapeutic immune response against HIV infection.

10. The composition of claim 9, the mixture further comprising:
(c) a third polynucleotide encoding a single HIV polypeptide consisting of HIV Nef polypeptide or an immunogenic fragment thereof.

11. The composition of claim 9, the mixture further comprising:
(d) a fourth polynucleotide encoding a single HIV polypeptide selected from a group consisting of HIV Pol polypeptide, HIV Tat polypeptide, HIV Rev polypeptide, HIV Vpr polypeptide, and immunogenic fragments thereof.

12. The composition of claim 9, wherein each polynucleotide encodes a different amino acid sequence.

13. The composition of claim 10, wherein each polynucleotide encodes a different amino acid sequence.

14. The composition of claim 9, wherein each polynucleotide is a different polynucleotide.

15. The composition of claim 9, wherein the polynucleotides are present in a viral vector.

16. The composition of claim 15, wherein the viral vector is an adenovirus vector.

17. A method for inducing a protective or therapeutic immune response against HIV comprising administering the composition of claim 1 to a subject in need thereof.

18. A method for inducing a protective or therapeutic immune response against HIV comprising administering the composition of claim 9 to a subject in need thereof

19. A method for inducing a protective or therapeutic immune response against HIV to a subject in need thereof, the method comprising:
(a) administering to the subject:
(i) a first polynucleotide encoding a single HIV polypeptide consisting of HIV Gag polypeptide or an immunogenic fragment thereof; and
(ii) a second polynucleotide encoding a single HIV polypeptide consisting of Vif polypeptide or an immunogenic fragment thereof;
wherein the polynucleotides are administered in an amount that is effective for inducing an immune response against HIV infection; and
(b) administering to the subject:
(i) a first viral vector expressing a single HIV polypeptide consisting of an HIV Gag polypeptide or an immunogenic fragment thereof; and
(ii) a second viral vector expressing a single HIV polypeptide consisting of HIV Vif polypeptide or an immunogenic fragment thereof;
wherein the vectors are administered in an amount that is effective for inducing an immune response against HIV infection.

20. The method of claim 19, wherein the polynucleotides are administered as a mixture of polynucleotides.

21. The method of claim 19, wherein the viral vectors are administered as a mixture of viral vectors.

22. The method of claim 19, wherein the polynucleotides are administered two or more subsequent times, waiting 1-3 weeks before a subsequent administration.

23. The method of claim 19, wherein the viral vectors are administered 1-6 months after administering the polynucleotides.

24. The method of claim 19, further comprising administering to the subject a third a polynucleotide encoding a single HIV polypeptide consisting of HIV Nef polypeptide or an immunogenic fragment thereof and administering to the subject a third viral vector expressing a single HIV polypeptide consisting of HIV Nef polypeptide or an immunogenic fragment thereof.
